(19) United States
(12) Patent Application Publication
(10) Pub. No.: US 2016/0033511 A1
(43) Pub. Date: Feb. 4, 2016

(54) METHODS AND COMPOSITIONS FOR DETECTING PANCREATIC CANCER

(71) Applicant: CREATICS LLC, Braintree, MA (US)

(72) Inventors: Lewis K. Pannell, Mobile, AL (US); Jana Rocker, Mobile, AL (US)

(21) Appl. No.: 14/773,969

(22) PCT Filed: Mar. 13, 2014
(86) PCT No.: PCT/US2014/026857

§ 371 Date: Sep. 9, 2015

Related U.S. Application Data
(60) Provisional application No. 61/780,574, filed on Mar. 13, 2013.

(51) Int. Cl. G01N 33/574

(52) U.S. Cl. CPC G01N 33/57438

(57) ABSTRACT

The present invention relates to non-invasive methods for the diagnosis and prognosis of pancreatic cancer. In some embodiments, such methods and compositions relate to particular pancreatic cancer biomarkers and combinations thereof.

### RELATED APPLICATIONS

This application claims priority to U.S. Provisional Application No. 61/780,574, filed Mar. 13, 2013, the entire contents of which are hereby incorporated by reference herein.

### FIELD OF THE INVENTION

The present invention relates to non-invasive methods for the diagnosis and prognosis of pancreatic cancer. In some embodiments, such methods and compositions relate to particular biomarkers and combinations thereof.

### BACKGROUND OF THE INVENTION

Disorders associated with the gastrointestinal (GI) and hepatobiliary tracts and the organs/tissues associated with the gastrointestinal tract include cancers such as gastric cancer, esophageal cancer, liver cancer, and pancreatic cancer. Pancreatic cancer (e.g., pancreatic adenocarcinoma), in particular, is a malignant growth of the pancreas that mainly occurs in the cells of the pancreatic ducts. This disease is the ninth most common form of cancer, yet it is the fourth and fifth leading cause of cancer deaths in men and women, respectively. Cancer of the pancreas is almost always fatal, with a five-year survival rate that is less than 3%.

The most common symptoms of pancreatic cancer include jaundice, abdominal pain, and weight loss, which, together with other presenting factors, are often nonspecific in nature. Thus, diagnosing pancreatic cancer at an early stage of tumor growth is often difficult and requires extensive diagnostic work-up, often times incidentally discovered during exploratory surgery. Endoscopic ultrasonography is an example of a non-surgical technique available for diagnosis of pancreatic cancer. However, reliable detection of small tumors, as well as differentiation of pancreatic cancer from focal pancreatitis, is difficult. The vast majority of patients with pancreatic cancer are presently diagnosed at a late stage when the tumor has already extended beyond the pancreas to invade surrounding organs and/or has metastasized extensively. Gold et al., Crit. Rev. Oncology/Hematology, 39:147-54 (2001), incorporated herein by reference in its entirety. Late detection of the disease is common with the majority of patients being diagnosed with advanced disease often resulting in death; only a minority of patients are detected with early stage disease.

Invasive techniques to diagnose disorders and diseases related to the gastrointestinal tract are inconvenient and expose a subject to significant risk. Examples of non-invasive methods to identify patients with disorders of the gastrointestinal tract or associated organs/tissues are described in PCT/US2011/051269 filed Sep. 12, 2011 entitled “NON-INVASIVE METHODS OF DETECTING PANCREATIC CANCER BIOMARKERS” which is incorporated by reference herein in its entirety. Nonetheless, there remains a need for additional methods for the diagnosis and prognosis of disorders such as pancreatic cancer.

### SUMMARY OF THE INVENTION

### Diagnostic Methods

In one aspect, the present invention is directed to a method of assessing whether a subject is afflicted with pancreatic cancer, the method including determining the level of at least one pancreatic cancer biomarker in a sample derived from said subject; and comparing the level of the pancreatic cancer biomarker with the level of the pancreatic cancer biomarker in a control sample, wherein a difference between the level of the pancreatic cancer biomarker derived from said subject and the pancreatic cancer biomarker in the control sample is an indication that the subject is afflicted with pancreatic cancer.

In various embodiments, the pancreatic cancer biomarker is CA19-9 or a protein comprising an amino acid sequence selected from the group consisting of SEQ ID NOs:1-31 and 38-793, or a fragment thereof. In certain embodiments, the pancreatic cancer biomarker is CA19-9 or a protein comprising an amino acid sequence selected from the group consisting of SEQ ID NOs:1-19, 47, 49-58, 206, 726, 729, 780 or 793, or a fragment thereof. In a particular embodiment, pancreatic cancer biomarker is a nucleotide sequence encoding the protein or the fragment thereof. In another embodiment, the pancreatic cancer biomarker is CA19-9.

In various embodiments, the sample is selected from the group consisting of a fecal sample, a gastrointestinal lavage fluid, and a combination thereof. In a particular embodiment, the sample is gastrointestinal lavage fluid.

In certain embodiments, the method includes determining the level of at least 2 pancreatic cancer biomarkers and comparing the level of each of the pancreatic cancer biomarkers to the respective level of the pancreatic cancer biomarkers in the control sample. In a particular embodiment, the method includes determining the level of at least 3, 4, 6, 7, 8, 9 or 10 pancreatic cancer biomarkers and comparing the level of each of the pancreatic cancer biomarkers to the respective level of the pancreatic cancer biomarkers in the control sample.

In one embodiment, the subject is a human.

In various embodiments, the method involves administering a lavage fluid and collecting the sample, for example, a gastrointestinal lavage fluid. In a particular embodiment, the lavage fluid is administered orally. In a particular embodiment, the lavage fluid includes an ingredient selected from the group consisting of polyethylene glycol, magnesium sulfate, sodium sulfate, potassium sulfate, magnesium citrate, ascorbic acid, sodium picosulfate, and bisacodyl. For example, the lavage fluid is selected from the group consisting of GOLYTELY, HALFLYTELY, NULYTELY, SUPREP, FLEET'S PHOSPHO-SODA, magnesium citrate, and their generic equivalents. In a particular embodiment, the method further includes partially purging the subject's gastrointestinal system and collecting gastrointestinal lavage fluid.

In one embodiment, the difference is a decrease in the level of the pancreatic cancer biomarker derived from said subject and the pancreatic cancer biomarker in the control sample, and wherein said decrease is an indication that the subject is afflicted with pancreatic cancer. For example, the pancreatic cancer biomarker may be a protein encoded by an amino acid sequence selected from the group consisting of SEQ ID NOs:1-16, 49, 55-58, 206 and 793, or a fragment thereof. In a particular embodiment, the level of the pancreatic cancer biomarker derived from said subject is at least 3 times less than the level of the pancreatic cancer biomarker in the control sample. Alternatively, the level of the pancreatic cancer biomarker derived from said subject is at least 5, 10 or 100 times less than the level of the pancreatic cancer biomarker in the control sample.

In another embodiment, the difference is an increase in the level of the pancreatic cancer biomarker derived from said subject and the pancreatic cancer biomarker in the control sample, and wherein said increase is an indication that the subject is afflicted with pancreatic cancer. For example, the pancreatic cancer biomarker may be a protein encoded by an amino acid sequence selected from the group consisting of SEQ ID NOs:17-19, 47, 726, 729 or 780, or a fragment thereof. Alternatively, the pancreatic cancer biomarker may be CA19-9. In various embodiments, the level of the pancreatic cancer biomarker derived from said subject is at least 3 times more than the level of the pancreatic cancer biomarker in the control sample. In a particular embodiment, the level of the pancreatic cancer biomarker derived from said subject is at least 5, 10 or 100 times more than the level of the pancreatic cancer biomarker in the control sample.

In one embodiment, the pancreatic cancer biomarker is derived from the pancreas. Alternatively, the pancreatic cancer biomarker may be derived from elsewhere in the gastrointestinal tract, for example the intestine.

In certain embodiments, the pancreatic cancer is selected from the group consisting of an exocrine pancreatic cancer, a pancreatic cystic neoplasm and a pancreatic endocrine cancer. For example, the pancreatic cancer may be an exocrine pancreatic cancer selected from the group consisting of pancreatic ductal adenocarcinoma (PDAC), adenosquamous carcinoma, squamous cell carcinoma, giant cell carcinoma, acinar cell carcinoma and small cell carcinoma. In a particular embodiment, the pancreatic cancer is pancreatic ductal adenocarcinoma. Alternatively, the pancreatic cancer may be a pancreatic endocrine tumor selected from the group consisting of insulinomas, glucagonomas, somatostatinomas, gastrinomas, VlPomas and non-secreting islet tumors of the pancreas.

In various embodiments, determining the level of said at least pancreatic cancer biomarker includes performing an immunoassay or a colorimetric assay. For example, the immunoassay may be a Western blot, an enzyme linked immunoabsorbent assay (ELISA), and a radioimmunoassay. In a particular embodiment, the immunoassay is an ELISA.

Alternatively, determining the level of said at least pancreatic cancer biomarker includes performing mass spectrometry.

Alternatively, determining the level of said at least pancreatic cancer biomarker includes applying said sample to a solid phase test strip or a flow-through strip including an agent which selectively binds to said pancreatic cancer biomarker; and detecting said pancreatic cancer biomarker bound to said agent on said solid phase test strip or said flow-through strip.

In particular embodiments, the method further involves comparing the level of the pancreatic cancer biomarker from the subject with the level of at least control polypeptide, or fragment thereof, or a nucleic acid encoding said at least control polypeptide, derived from the sample. For example, the control polypeptide may be a non-pancreatic polypeptide that originates in the gastrointestinal tract. In a particular embodiment, the control polypeptide comprises an amino acid sequence selected from the group consisting of SEQ ID NOs:27, 32-40, 45, 54, 59 and 59, or a fragment thereof.

### Prognostic Methods

In another aspect, the present invention is directed to a method of assessing the progression of pancreatic cancer in a subject afflicted with pancreatic cancer, by determining the level of at least one pancreatic cancer biomarker in a sample derived from said subject; and comparing the level of the pancreatic cancer biomarker with the level of the pancreatic cancer biomarker in a control sample, wherein a decrease in the level of the pancreatic cancer biomarker derived from said subject and the pancreatic cancer biomarker in the control sample is an indication that the pancreatic cancer will progress rapidly; and wherein an increase in the level of the pancreatic cancer biomarker derived from said subject and the pancreatic cancer biomarker in the control sample is an indication that the pancreatic cancer will progress slowly or will regress; optionally, wherein the pancreatic cancer biomarker is CA19-9, a protein encoded by an amino acid sequence selected from the group consisting of SEQ ID NOs:1-31 or 39-793, a fragment thereof, or a nucleotide sequence encoding the protein or fragment thereof. For example, the pancreatic cancer biomarker may be a protein encoded by an amino acid sequence selected from the group consisting of SEQ ID NOs:1-16, 49, 55-58, 206 and 793, a fragment thereof or a nucleotide sequence encoding the protein or the fragment thereof.

In another aspect, the present invention is directed to a method of assessing the progression of pancreatic cancer in a subject afflicted with pancreatic cancer, by determining the level of at least one pancreatic cancer biomarker in a sample derived from said subject; and comparing the level of the pancreatic cancer biomarker with the level of the pancreatic cancer biomarker in a control sample, wherein an increase in the level of the pancreatic cancer biomarker derived from said subject and the pancreatic cancer biomarker in the control sample is an indication that the pancreatic cancer will progress rapidly; and wherein a decrease in the level of the pancreatic cancer biomarker derived from said subject and the pancreatic cancer biomarker in the control sample is an indication that the pancreatic cancer will progress slowly or will regress; optionally, wherein the pancreatic cancer biomarker is CA19-9, a protein encoded by an amino acid sequence selected from the group consisting of SEQ ID NOs:1-31 or 39-793, a fragment thereof, or a nucleotide sequence encoding the protein or fragment thereof. For example, the pancreatic cancer biomarker may be CA19-9 or a protein encoded by an amino acid sequence selected from the group consisting of SEQ ID NOs:17-19, 47, 726, 729 or 780, a fragment thereof, or a nucleotide sequence encoding the protein or the fragment thereof.

In various embodiments of the foregoing aspects, the sample is selected from the group consisting of a fecal sample, a gastrointestinal lavage fluid, and a combination thereof. In a particular embodiment, the sample is gastrointestinal lavage fluid.

In certain embodiments of the foregoing aspects, the method includes determining the level of at least 2 pancreatic cancer biomarkers and comparing the level of each of the pancreatic cancer biomarkers to the respective level of the pancreatic cancer biomarkers in the control sample. In a particular embodiment, the method includes determining the level of at least 3, 4, 6, 7, 8, 9 or 10 pancreatic cancer biomarkers and comparing the level of each of the pancreatic cancer biomarkers to the respective level of the pancreatic cancer biomarkers in the control sample.

In one embodiment, the subject is a human.

In various embodiments, the method involves administering a lavage fluid and collecting the sample, for example, a gastrointestinal lavage fluid. In a particular embodiment, the lavage fluid is administered orally. In a particular embodiment, the lavage fluid includes an ingredient selected from the group consisting of polyethylene glycol, magnesium sulfate, sodium sulfate, potassium sulfate, magnesium citrate, ascorbic acid, sodium picosulfate, and bisacodyl. For example, the lavage fluid is selected from the group consisting of GOLYTELY, HALFLYTELY, NULYTELY, SUPREP, FLEET'S PHOSPHO-SODA, magnesium citrate, and their generic equivalents. In a particular embodiment, the method further includes partially purging the subject's gastrointestinal system and collecting gastrointestinal lavage fluid.

In certain embodiments of the foregoing aspects, the decrease is at least 3, 5, 10 or 100 times less than the level of pancreatic cancer biomarker in the control sample. Alternatively, the increase is at least 3, 5, 10 or 100 times more than the level of pancreatic cancer biomarker in the control sample.

In one embodiment, the pancreatic cancer biomarker is derived from the pancreas. Alternatively, the pancreatic cancer biomarker may be derived from elsewhere in the gastrointestinal tract, for example the intestine.

In certain embodiments, the pancreatic cancer is selected from the group consisting of an exocrine pancreatic cancer, a pancreatic cystic neoplasm and a pancreatic endocrine cancer. For example, the pancreatic cancer may be an exocrine pancreatic cancer selected from the group consisting of pancreatic ductal adenocarcinoma (PDAC), adenosquamous carcinoma, squamous cell carcinoma, giant cell carcinoma, acinar cell carcinoma and small cell carcinoma. In a particular embodiment, the pancreatic cancer is pancreatic ductal adenocarcinoma. Alternatively, the pancreatic cancer may be a pancreatic endocrine tumor selected from the group consisting of insulinomas, glucagonomas, somatostatinomas, gastrinomas, VlPomas and non-secreting islet tumors of the pancreas.

In various embodiments, determining the level of said at least pancreatic cancer biomarker includes performing an immunoassay or a colorimetric assay. For example, the immunoassay may be a Western blot, an enzyme linked immunoabsorbent assay (ELISA), and a radioimmunoassay. In a particular embodiment, the immunoassay is an ELISA.

Alternatively, determining the level of said at least pancreatic cancer biomarker includes performing mass spectrometry.

Alternatively, determining the level of said at least pancreatic cancer biomarker includes applying said sample to a solid phase test strip or a flow-through strip including an agent which selectively binds to said pancreatic cancer biomarker; and detecting said pancreatic cancer biomarker bound to said agent on said solid phase test strip or said flow-through strip.

In particular embodiments, the method further involves comparing the level of the pancreatic cancer biomarker from the subject with the level of at least control polypeptide, or fragment thereof, or a nucleic acid encoding said at least control polypeptide, derived from the sample. For example, the control polypeptide may be a non-pancreatic polypeptide that originates in the gastrointestinal tract. In a particular embodiment, the control polypeptide comprises an amino acid sequence selected from the group consisting of SEQ ID NOs:27, 32-40, 45, 54, 59 and 59, or a fragment thereof.

### Methods of Monitoring Treatment and Method of Treating

In another aspect, the present invention is directed to a method of monitoring the efficacy of treatment of pancreatic cancer in a subject suffering from pancreatic cancer, by determining the level of at least one pancreatic cancer biomarker in a sample derived from said subject, wherein said subject has been previously exposed to treatment for pancreatic cancer; and comparing the level of the pancreatic cancer biomarker with the level of the pancreatic cancer biomarker in a control sample, wherein a decrease in the level of the pancreatic cancer biomarker derived from said subject and the pancreatic cancer biomarker in the control sample is an indication that the treatment is not efficacious; and wherein an increase in the level of the pancreatic cancer biomarker derived from said subject and the pancreatic cancer biomarker in the control sample is an indication that the pancreatic cancer is efficacious; optionally, wherein the pancreatic cancer biomarker is CA19-9, a protein encoded by an amino acid sequence selected from the group consisting of SEQ ID NOs:1-31 or 39-793, a fragment thereof, or a nucleotide sequence encoding the protein or fragment thereof. For example, the pancreatic cancer biomarker may be a protein encoded by an amino acid sequence selected from the group consisting of SEQ ID NOs:1-16, 49, 55-58, 206 and 793, a fragment thereof or a nucleotide sequence encoding the protein or the fragment thereof.

In another aspect, the present invention is directed to a method of monitoring the efficacy of treatment of pancreatic cancer in a subject suffering from pancreatic cancer, by determining the level of at least one pancreatic cancer biomarker in a sample derived from said subject, wherein said subject has been previously exposed to treatment for pancreatic cancer; and comparing the level of the pancreatic cancer biomarker with the level of the pancreatic cancer biomarker in a control sample, wherein an increase in the level of the pancreatic cancer biomarker derived from said subject and the pancreatic cancer biomarker in the control sample is an indication that the treatment is not efficacious; and wherein a decrease in the level of the pancreatic cancer biomarker derived from said subject and the pancreatic cancer biomarker in the control sample is an indication that the pancreatic cancer is efficacious; optionally, wherein the pancreatic cancer biomarker is CA19-9, a protein encoded by an amino acid sequence selected from the group consisting of SEQ ID NOs:1-31 or 39-793, a fragment thereof, or a nucleotide sequence encoding the protein or fragment thereof. For example, the pancreatic cancer biomarker may be CA19-9 or a protein encoded by an amino acid sequence selected from the group consisting of SEQ ID NOs:17-19, 47, 726, 729 or 780, a fragment thereof, or a nucleotide sequence encoding the protein or the fragment thereof.

In another aspect, the present invention is directed to a method of treating a subject having pancreatic cancer, by determining the level of at least one pancreatic cancer biomarker in a sample derived from said subject; and comparing the level of the pancreatic cancer biomarker with the level of the pancreatic cancer biomarker in a control sample, wherein a difference between the level of the pancreatic cancer biomarker derived from said subject and the pancreatic cancer biomarker in the control sample is an indication that the subject is afflicted with pancreatic cancer; and exposing said subject to therapeutically effective treatment, thereby treating the subject having pancreatic cancer; optionally, wherein the pancreatic cancer biomarker is CA19-9, a protein encoded by an amino acid sequence selected from the group consisting of SEQ ID NOs:1-31 or 39-793, a fragment thereof, or a nucleotide sequence encoding the protein or fragment thereof.

In various embodiments of the foregoing aspects of the invention, the treatment is selected from the group consisting of surgery, radiation, chemotherapy or a combination thereof. For example, surgery may comprise the Whipple procedure, total pancreatectomy, distal pancreatectomy, surgical biliary bypass, endoscopic stent placement or gastric bypass. Alternatively, treatment may consist of administration of agents for treatment including, for example, tyrosine kinase inhibitors (TKIs) such as Erlotinib.

In various embodiments of the foregoing aspects, the sample is selected from the group consisting of a fecal sample, a gastrointestinal lavage fluid, and a combination thereof. In a particular embodiment, the sample is gastrointestinal lavage fluid.

In certain embodiments of the foregoing aspects, the method includes determining the level of at least 2 pancreatic cancer biomarkers and comparing the level of each of the pancreatic cancer biomarkers to the respective level of the pancreatic cancer biomarkers in the control sample. In a particular embodiment, the method includes determining the level of at least 3, 4, 6, 7, 8, 9 or 10 pancreatic cancer biomarkers and comparing the level of each of the pancreatic cancer biomarkers to the respective level of the pancreatic cancer biomarkers in the control sample.

In one embodiment, the subject is a human.

In various embodiments, the method involves administering a lavage fluid and collecting the sample, for example, a gastrointestinal lavage fluid. In a particular embodiment, the lavage fluid is administered orally. In a particular embodiment, the lavage fluid includes an ingredient selected from the group consisting of polyethylene glycol, magnesium sulfate, sodium sulfate, potassium sulfate, magnesium citrate, ascorbic acid, sodium picosulfate, and bisacodyl. For example, the lavage fluid is selected from the group consisting of GOLYTELY, HALFLYTELY, NULYTELY, SUPREP, FLEET'S PHOSPHO-SODA, magnesium citrate, and their generic equivalents. In a particular embodiment, the method further includes partially purging the subject's gastrointestinal system and collecting gastrointestinal lavage fluid.

In certain embodiments of the foregoing aspects, the decrease is at least 3, 5, 10 or 100 times less than the level of pancreatic cancer biomarker in the control sample. Alternatively, the increase is at least 3, 5, 10 or 100 times more than the level of pancreatic cancer biomarker in the control sample.

In one embodiment, the pancreatic cancer biomarker is derived from the pancreas. Alternatively, the pancreatic cancer biomarker may be derived from elsewhere in the gastrointestinal tract, for example the intestine.

In certain embodiments, the pancreatic cancer is selected from the group consisting of an exocrine pancreatic cancer, a pancreatic cystic neoplasm and a pancreatic endocrine cancer. For example, the pancreatic cancer may be an exocrine pancreatic cancer selected from the group consisting of pancreatic ductal adenocarcinoma (PDAC), adenosquamous carcinoma, squamous cell carcinoma, giant cell carcinoma, acinar cell carcinoma and small cell carcinoma. In a particular embodiment, the pancreatic cancer is pancreatic ductal adenocarcinoma. Alternatively, the pancreatic cancer may be a pancreatic endocrine tumor selected from the group consisting of insulinomas, glucagonomas, somatostatinomas, gastrinomas, VlPomas and non-secreting islet tumors of the pancreas.

In various embodiments, determining the level of said at least pancreatic cancer biomarker includes performing an immunoassay or a colorimetric assay. For example, the immunoassay may be a Western blot, an enzyme linked immunoabsorbent assay (ELISA), and a radioimmunoassay. In a particular embodiment, the immunoassay is an ELISA.

Alternatively, determining the level of said at least pancreatic cancer biomarker includes performing mass spectrometry.

Alternatively, determining the level of said at least pancreatic cancer biomarker includes applying said sample to a solid phase test strip or a flow-through strip including an agent which selectively binds to said pancreatic cancer biomarker; and detecting said pancreatic cancer biomarker bound to said agent on said solid phase test strip or said flow-through strip.

In particular embodiments, the method further involves comparing the level of the pancreatic cancer biomarker from the subject with the level of at least control polypeptide, or fragment thereof, or a nucleic acid encoding said at least control polypeptide, derived from the sample. For example, the control polypeptide may be a non-pancreatic polypeptide that originates in the gastrointestinal tract. In a particular embodiment, the control polypeptide comprises an amino acid sequence selected from the group consisting of SEQ ID NOs:27, 32-40, 45, 54, 59 and 59, or a fragment thereof.

### Kit

In a further aspect, the present invention is directed to a kit for determining the presence, absence or progression of pancreatic cancer in a subject including an agent that selectively binds to at least one pancreatic cancer biomarker.

For example, the pancreatic cancer biomarker may be CA19-9 or a protein having an amino acid sequence selected from the group consisting of SEQ ID NOs:1-31 or 39-793, or a fragment thereof. In a particular embodiment, the pancreatic cancer biomarker is CA19-9 or a protein comprising an amino acid sequence selected from the group consisting of SEQ ID NOs:1-19, 47, 49-58, 206, 726, 729, 780 or 793. In a particular embodiment, the pancreatic cancer biomarker is a nucleotide sequence encoding the foregoing protein.

In a particular embodiment, the kit includes at least two agents that selectively bind to at least one pancreatic cancer biomarker. For example, the kit can include at least three, four or five agents that selectively bind to at least one pancreatic cancer biomarker. In a particular embodiments, the agent is an antibody or antigen-binding fragment thereof. In certain embodiments the agent is attached to a solid support, such as a solid phase test strip or a flow-through test strip. In further embodiments, the kit includes a detectable agent which selectively binds to said pancreatic cancer biomarker.

In various embodiments, the kit includes a lavage fluid for oral administration to a subject and, optionally, a vessel for collecting the gastrointestinal lavage fluid from the subject.

### Compositions

Some compositions and methods provided herein include an isolated polypeptide consisting essentially of an amino acid sequence selected from the group consisting of a polypeptide comprising, consisting essentially of, or consisting of SEQ ID NOs:1-31 or 39-793 or a fragment thereof, wherein said polypeptide is differentially expressed in cancer.

Some compositions and methods provided herein include an isolated nucleic acid encoding a polypeptide consisting essentially of an amino acid sequence selected from the group consisting of a polypeptide comprising, consisting essentially of, or consisting of SEQ ID NOs:1-31 or 39-793 or a fragment thereof, wherein said polypeptide is differentially expressed in cancer.

Some compositions and methods provided herein include an isolated polypeptide consisting of an amino acid sequence selected from the group consisting of a polypeptide comprising, consisting essentially of, or consisting of SEQ ID NOs:1-31 or 39-793 or a fragment thereof, wherein said polypeptide is differentially expressed in cancer.

Some compositions and methods provided herein include an isolated nucleic acid encoding a polypeptide consisting of an amino acid sequence selected from the group consisting of a polypeptide comprising, consisting essentially of, or consisting of SEQ ID NOs:1-31 or 39-793 or a fragment thereof, wherein said polypeptide is differentially expressed in cancer.

Some compositions and methods provided herein include an isolated agent that selectively binds to an isolated polypeptide consisting essentially of an amino acid sequence selected from the group consisting of a polypeptide comprising, consisting essentially of, or consisting of SEQ ID NOs:1-31 or 39-793 or a fragment thereof, wherein said polypeptide is differentially expressed in cancer. In some embodiments, the agent comprises an antibody or antigen-binding fragment thereof.

Some compositions and methods provided herein include an isolated agent that selectively binds to an isolated polypeptide consisting of an amino acid sequence selected from the group consisting of a polypeptide comprising, consisting essentially of, or consisting of SEQ ID NOs:1-31 or 39-793 or a fragment thereof, wherein said polypeptide is differentially expressed in cancer. In some embodiments, the agent comprises an antibody or antigen-binding fragment thereof.

### BRIEF DESCRIPTION OF DRAWINGS

FIG. 1 depicts the processing of gastrointestinal lavage fluid samples obtained from subjects prior to mass spectrometry analysis, as described in Example 4.

FIG. 2 depicts the processing of the same control sample six times to assess variation in key proteins. The results reflect that the methodology results in data showing little variation and thus, the method is highly reproducible, as described in Example 5.

FIG. 3 depicts a volcano plot of the intensity values prior to “roll up” of proteins in the gastrointestinal lavage fluid of subjects with pancreatic ductal adenocarcinoma in the head of the pancreas versus control, as described in Example 5.

FIG. 4 depicts a volcano plot of the intensity values after “roll up” of proteins in the gastrointestinal lavage fluid of subjects with pancreatic ductal adenocarcinoma in the head of the pancreas versus control, as described in Example 5.

### DETAILED DESCRIPTION

The present invention is based, at least in part, on the unexpected discovery that particular pancreatic cancer biomarkers, for example, proteins secreted from the pancreas or other non-pancreatic sources in the gastrointestinal tract, are found at modified levels, for example, at decreased or increased levels, in gastrointestinal lavage fluid or fecal matter of a subject having pancreatic cancer. Indeed, the inventors have identified that gastrointestinal lavage fluid or fecal matter provide a unique opportunity to assess the presence of pancreatic cancer in a non-invasive, rapid and efficient manner.

As a result, the present invention provides methods for diagnosing pancreatic cancer by assessing levels of pancreatic cancer biomarkers in gastrointestinal lavage fluid or fecal matter derived from a subject.

Moreover, the present invention is further predicated, at least in part, on the discovery that relative changes in the levels of proteins or polypeptides that originate from the pancreas, and other sources, compared to relative changes in the levels of particular proteins or polypeptides that originate from other gastrointestinal (GI) systems can be used to detect pancreatic cancer. Accordingly, the levels of particular proteins or polypeptides originating from non-pancreatic sources can be useful as control levels for assessing whether a subject is suffering from pancreatic cancer.

### DEFINITIONS

Unless otherwise defined herein, scientific and technical terms used in connection with the present invention shall have the meanings that are commonly understood by those of ordinary skill in the art. The meaning and scope of the terms should be clear. However, in the event of any latent ambiguity, definitions provided herein take precedent over any dictionary or extrinsic definition. Further, unless otherwise required by context, singular terms, for example, those characterized by “a” or “an”, shall include pluralities. In this application, the use of “or” means “and/or”, unless stated otherwise. Furthermore, the use of the term “including,” as well as other forms of the term, such as “includes” and “included”, is not limiting. Also, terms such as “element” or “component” encompass both elements and components comprising one unit and elements and components that comprise more than one unit unless specifically stated otherwise.

The term “comprising” as used herein is synonymous with “including,” “containing,” or “characterized by,” and is inclusive or open-ended and does not exclude additional, unrecited elements or method steps. As used herein “consisting essentially of” refers to a peptide or polypeptide which includes an amino acid sequence of the proteins provided herein, for example, SEQ ID NOs:1-793, along with additional amino acids at the carboxyl and/or amino terminal ends where the additional amino acids do not materially alter the ability of the peptide or polypeptide to be diagnostically useful for the relevant type or types of cancer. For example, in some embodiments, a peptide or polypeptide “consisting essentially of” a particular sequence may include an amino acid sequence of the proteins provided herein, for example SEQ ID NOs:1-793, along with no more than 1, no more than 2, no more than 3, no more than 4, no more than 5, no more than 6, no more than 7, no more than 8, no more than 9, or no more than 10 additional amino acid(s) at the carboxyl and/or amino terminal ends of a polypeptide provided herein, for example, one of SEQ ID NOs:1-793.

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

As used herein, the term “subject” refers to human and non-human animals, including veterinary subjects. The term “non-human animal” includes all vertebrates, e.g., mammals and non-mammals, such as non-human primates, mice, rabbits, sheep, dog, cat, horse, cow, chickens, amphibians, and reptiles. In a preferred embodiment, the subject is a human.

The terms “cancer” or “tumor” are well known in the art and refer to the presence, e.g., in a subject, of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells may exist alone within a subject, or may be non-tumorigenic cancer cells, such as leukemia cells. As used herein, the term “cancer” includes pre-malignant as well as malignant cancers.

As used herein, “pancreas” in reference to an organ refers to a collection of a plurality of cell types held together by connective tissue, such that the plurality of cells include but are not limited to acini calls, ductal cells and islet cells. The “acini” produce many of the enzymes, such as lipase, which are needed to digest food in the duodenum. The enzymes produced by the acini are carried to the duodenum by small channels called ducts. Typically, ductal cells are held in place by connective tissue in close proximity to vascular cells and nerve cells. Islets of Langerhans are typically embedded between exocrine acini units of the pancreas. Examples of islet endocrine cells are Alpha cells that secrete glucagon which counters the action of insulin while Beta cells secrete insulin, which helps control carbohydrate metabolism.

As used herein, a subject who is “afflicted with pancreatic cancer” is one who is clinically diagnosed with such a cancer by a qualified clinician (for example, by the methods of the present invention), or one who exhibits one or more signs or symptoms (for example, reduced levels of a pancreatic cancer biomarker in gastrointestinal lavage fluid or fecal matter) of such a cancer and is subsequently clinically diagnosed with such a cancer by a qualified clinician (for example, by the methods of the present invention). A non-human subject that serves as an animal model of pancreatic cancer may also fall within the scope of the term a subject “afflicted with pancreatic cancer.”

As used herein, the term “pancreatic cancer” refers to the art recognized disease and includes cancers that originate in the tissue that comprises a pancreas. In various embodiments, the pancreatic cancer is an exocrine pancreatic cancer, a pancreatic cystic neoplasm or a pancreatic endocrine tumor.

In a particular embodiment, the pancreatic cancer is an exocrine pancreatic cancer selected from the group consisting of pancreatic ductal adenocarcinoma (PDAC), adenosquamous carcinoma, squamous cell carcinoma, giant cell carcinoma, acinar cell carcinoma and small cell carcinoma.

In a particular embodiment, the pancreatic cancer is a ductal adenocarcinoma, e.g., resectable pancreatic ductal adenocarcinoma (PDAC), which arises within the exocrine component of the pancreas. As used herein, “adenocarcinoma” refers to a cancerous tumor as opposed to an “adenoma” which refers to a benign (non-cancerous) tumor made up of cells that form glands (collections of cells surrounding an empty space). As used herein, “pancreatic ductal adenocarcinoma cell” refers to a cancerous cell that had the capability to form or originated from the ductal lining of the pancreas. A pancreatic ductal adenocarcinoma cell may be found within the pancreas forming a gland, or found within any organ as a metastasized cell or found within the blood stream of lymphatic system. As used herein, “ductal cell”, in reference to a pancreas, refers to any cell that forms or has the capability to form or originated from the ductal lining of ducts within and exiting from the pancreas.

In another embodiment, the pancreatic cancer is a pancreatic endocrine tumor, also known as islet cell tumors, pancreas endocrine tumors (PETs) and pancreatic neuroendocrine tumors (PNETs), which arises from islet cells. In a particular embodiment, the pancreatic cancer is an endocrine pancreatic cancer selected from the group consisting of insulinomas (i.e., arising from insulin-producing cells), glucagonomas (i.e., arising from glucagon-producing cells), somatostatinomas (i.e., arising from somatostatin-making cells), gastrinomas (i.e., arising from a gastrin-producing cells), VIPomas (arising from vasoactive intestinal peptide-making cells) and non-secreting islet tumors of the pancreas.

As used herein, the term “pancreatic cancer biomarker” refers to a protein or non-proteinaceous substance which is differentially present in gastrointestinal lavage fluid or fecal matter in subjects afflicted with pancreatic cancer as compared to subjects without pancreatic cancer. In particular embodiments, the protein is derived from the pancreas. In other embodiments, the protein is derived from non-pancreatic sources in the gastrointestinal tract, e.g., the intestine. In various embodiments, the pancreatic cancer biomarker is a protein selected from the group consisting of SEQ ID NOs:1-31 or 39-793. In a particular embodiment, the pancreatic cancer biomarker is a protein selected from the group consisting of SEQ ID NOs:1-19, 47, 49, 55-58, 206, 726, 729, 780 or 793. As used herein, isoforms and mature forms of the proteins specifically identified herein are also intended to be encompassed by the methods of the present invention. In addition, fragments of the proteins specifically identified herein are also intended to be encompassed by the methods of the present invention. As used herein, the term “fragment” refers to a fragment of a protein that preserves at least the structure, e.g., a portion of the amino acid sequence, or at least one function, e.g., activity, of the protein from which it is derived.

Alternatively, the pancreatic cancer biomarker may refer to a non-proteinaceous substance. For example, the pancreatic cancer may be CA19-9. As used herein, CA19-9, also known as carbohydrate antigen 19-9, cancer antigen 19-9 or sialylated Lewis (a) antigen) is a tumor marker often assayed in serum or blood.

The “level” of pancreatic cancer biomarker, as used herein, refers to the level of the pancreatic cancer biomarker in gastrointestinal lavage fluid or fecal matter as determined using a method for the measurement of levels of protein or non-proteinaceous substances. Such methods include, for example, electrophoresis, capillary electrophoresis, high performance liquid chromatography (HPLC), thin layer chromatography (TLC), hyperdiffusion chromatography, fluid or gel precipitation reactions, absorption spectroscopy, a colorimetric assays, spectrophotometric assays, flow cytometry, immunodiffusion (single or double), solution phase assay, immunoelectrophoresis, Western blotting, radioimmunoassay (RIA), enzyme-linked immunosorbent assays (ELISAs), immunofluorescent assays, and electrochemiluminescence immunoassay (exemplified below), and the like. In a preferred embodiment, the level is determined using an ELISA based assay.

The term “sample” as used herein refers to a collection of similar fluids, cells, or tissues isolated from a subject, as well as fluids, cells, or tissues present within a subject. In preferred embodiments, the sample is a biological fluid containing a pancreatic cancer biomarker. Biological fluids are typically liquids at physiological temperatures and may include naturally occurring fluids present in, withdrawn from, expressed or otherwise extracted from a subject or biological source. Certain biological fluids derive from particular tissues, organs or localized regions and certain other biological fluids may be more globally or systemically situated in a subject or biological source. Examples of biological fluids include gastrointestinal lavage fluid, fecal matter, blood, serum and serosal fluids, plasma, semen, pancreatic fluid, bile, lymph, urine, cerebrospinal fluid, saliva, ocular fluids, cystic fluid, tear drops, sputum, mucosal secretions of the secretory tissues and organs, vaginal secretions, gynecological fluids, ascites fluids such as those associated with non-solid tumors, fluids of the pleural, pericardial, peritoneal, abdominal and other body cavities, fluids collected by bronchial lavage and the like. In a particular embodiment, the sample is gastrointestinal lavage fluid or fecal matter.

In certain embodiments, the sample is a biological fluid formed of a liquid solution contacted with a subject or biological source. In a particular embodiment, the sample is a gastrointestinal lavage fluid.

In one embodiment, the sample is removed or obtained from the subject, for example, according to the methods described herein. In another embodiment, the sample is present within the subject.

In some embodiments, only a portion of the sample is subjected to an assay for determining the level of the pancreatic cancer biomarker, or various portions of the sample are subjected to various assays for determining the level of the pancreatic cancer biomarker. Also, in many embodiments, the sample may be pre-treated by physical or chemical means prior to the assay. For example, in embodiments discussed in more detail in the Examples section, samples, for example, gastrointestinal lavage fluid samples, were subjected to centrifugation, extraction (e.g., chloroform extraction), precipitation (e.g., methanol, chloroform and/or water precipitation), and digestion (e.g., with trypsin) prior to assaying the samples for the pancreatic cancer biomarker protein. Such techniques serve to enhance the accuracy, reliability and reproducibility of the assays of the present invention.

The term “control sample,” as used herein, refers to any clinically relevant control sample, including, for example, a sample from a healthy subject not afflicted with pancreatic cancer, a sample from a subject having a less severe or slower progressing pancreatic cancer than the subject to be assessed, a sample from a subject having some other type of cancer or disease, and the like. A control sample may include a sample derived from one or more subjects. A control sample may also be a sample made at an earlier time point from the subject to be assessed. For example, the control sample could be a sample taken from the subject to be assessed before the onset of pancreatic cancer, at an earlier stage of disease, or before the administration of treatment or of a portion of treatment. The control sample may also be a sample from an animal model, or from a tissue or cell lines derived from the animal model, of the pancreatic cancer. The level of pancreatic cancer biomarker in a control sample that consists of a group of measurements may be determined based on any appropriate statistical measure, such as, for example, measures of central tendency including average, median, or modal values.

The term “control level” refers to an accepted or pre-determined level of pancreatic cancer biomarker which is used to compare with the level of pancreatic cancer biomarker in a sample derived from a subject. In one embodiment, the control level of pancreatic cancer biomarker is based on the level of pancreatic cancer biomarker in sample(s) from a subject(s) having slow disease progression. In another embodiment, the control level of pancreatic cancer biomarker is based on the level in a sample from a subject(s) having rapid disease progression. In another embodiment, the control level of pancreatic cancer biomarker is based on the level of pancreatic cancer biomarker in a sample(s) from an unaffected, i.e., non-diseased, subject(s), i.e., a subject who does not have pancreatic cancer. In yet another embodiment, the control level of pancreatic cancer biomarker is based on the level of pancreatic cancer biomarker in a sample from a subject(s) prior to the administration of a therapy for pancreatic cancer. In another embodiment, the control level of pancreatic cancer biomarker is based on the level of pancreatic cancer biomarker in a sample(s) from a subject(s) having pancreatic cancer that is not contacted with a test compound. In another embodiment, the control level of pancreatic cancer biomarker is based on the level of pancreatic cancer biomarker in a sample(s) from a subject(s) not having pancreatic cancer that is contacted with a test compound. In one embodiment, the control level of pancreatic cancer biomarker is based on the level of pancreatic cancer biomarker in a sample(s) from an animal model of pancreatic cancer, a cell, or a cell line derived from the animal model of pancreatic cancer.

In one embodiment, the control is a standardized control, such as, for example, a control which is predetermined using an average of the levels of pancreatic cancer biomarker from a population of subjects having no pancreatic cancer. In still other embodiments of the invention, a control level of pancreatic cancer biomarker is based on the level of pancreatic cancer biomarker in a non-cancerous sample(s) derived from the subject having pancreatic cancer.

As used herein, “a difference” between the level of pancreatic cancer biomarker in a sample from a subject (i.e., gastrointestinal lavage fluid) and the level of pancreatic cancer biomarker in a control sample refers broadly to any clinically relevant and/or statistically significant difference in the level of pancreatic cancer biomarker in the two samples. In an exemplary embodiment, the difference is determined as set forth in the Examples set forth below.

In other embodiments, the difference must be greater than the limits of detection of the method for determining the level of pancreatic cancer biomarker. It is preferred that the difference be at least greater than the standard error of the assessment method, and preferably a difference of at least about 2-, about 3-, about 4-, about 5-, about 6-, about 7-, about 8-, about 9-, about 10-, about 15-, about 20-, about 25-, about 100-, about 500-, about 1000-fold or greater than the standard error of the assessment method. The difference may be assessed by any appropriate comparison, including any appropriate parametric or nonparametric descriptive statistic or comparison. For example, “an increase” in the level of pancreatic cancer biomarker may refer to a level in a test sample, e.g., gastrointestinal lavage fluid, that is about two, and more preferably about three, about four, about five, about six, about seven, about eight, about nine, about ten or more times more than the level of pancreatic cancer biomarker in the control sample. An increase may also refer to a level in a test sample that is preferably at least about 1.5, and more preferably about two, about three, about four, about five or more standard deviations above the average level of pancreatic cancer biomarker in the control sample. Likewise, “a decrease” in the level of pancreatic cancer biomarker may refer to a level in a test sample that is preferably at least about two, and more preferably about three, about four, about five, about six, about seven, about eight, about nine, about ten or more times less than the level of pancreatic cancer biomarker in the control sample. A decrease may also refer to a level in a test sample that is preferably at least about 1.5, and more preferably about two, about three, about four, about five or more standard deviations below the average level of pancreatic cancer biomarker in the control sample.

### Biological Samples

As set forth herein, a sample for use in the methods of the present invention refers to a collection of similar fluids, cells, or tissues isolated from a subject, as well as fluids, cells, or tissues present within a subject. In preferred embodiments the sample is a biological fluid containing a pancreatic cancer biomarker protein. Examples of biological fluids include gastrointestinal lavage fluid, fecal matter, blood, serum and serosal fluids, plasma, semen, pancreatic fluid, bile, lymph, urine, cerebrospinal fluid, saliva, ocular fluids, cystic fluid, tear drops, sputum, mucosal secretions of the secretory tissues and organs, vaginal secretions, gynecological fluids, ascites fluids such as those associated with non-solid tumors, fluids of the pleural, pericardial, peritoneal, abdominal and other body cavities, fluids collected by bronchial lavage and the like.

In a particular embodiment, the sample is a biological fluid originating from the gastrointestinal tract (GI tract). As is well known in the art, the gastrointestinal tract includes the upper gastrointestinal tract and lower gastrointestinal tract. The upper gastrointestinal tract includes the oral or buccal cavity, esophagus, stomach and duodenum. The lower gastrointestinal tract includes the jejunum, ileum and the large intestine and the anus. The large intestine includes the appendix, cecum, colon, and rectum. Organs and tissues associated with the gastrointestinal tract include structures outside the gastrointestinal tract. Examples of such structures include accessory digestive organs such as salivary glands, e.g., parotid salivary glands, submandibular salivary glands, and sublingual salivary glands, pancreas, e.g., exocrine pancreas, gallbladder, bile duct, and liver. More examples of structures associated with the gastrointestinal tract and outside the gastrointestinal tract include the pancreatic duct, biliary tree, and bile duct.

In a particular embodiment, the biological sample is gastrointestinal lavage fluid. In some embodiments, a biological sample includes a gastrointestinal lavage fluid. Generally, a lavage fluid can be orally administered to a subject, the oral lavage fluid passes through the gastrointestinal tract of the subject, and the resulting gastrointestinal lavage fluid is collected from the subject. Alternative lavage methods include direct washing of the cavity with a lavage fluid during surgery or endoscopy or washing via the rectum by means of enemas or colonic irrigation. As noted above, gastrointestinal lavage fluid provides a cleaner sampling of the gastrointestinal tract than the examination of feces/stool samples. Gastrointestinal lavage fluids appear to mitigate variability related to food intake, type and digestive status.

Some embodiments described herein include analysis of a gastrointestinal lavage fluid for detecting a pancreatic cancer biomarker for screening, disease detection, diagnosis, prognosis, response to treatment, selection of treatment and personalized medicine for diseases and pathological conditions of the gastrointestinal tract or associated organs/tissues, such as pancreatic cancer.

### Methods for Obtaining a Gastrointestinal Lavage Fluid

In certain embodiments of the present invention, a gastrointestinal lavage fluid sample is obtained from a subject. For example, a gastrointestinal lavage fluid may be obtained as described in International Application No. PCT/US2011/051269, filed on Sep. 12, 2011 and entitled “NON-INVASIVE METHODS OF DETECTING PANCREATIC CANCER BIOMARKERS”, the entire contents of which are hereby incorporated by reference herein. Some methods of obtaining a gastrointestinal lavage fluid include orthograde colonic lavage. Orthograde lavage can include orally administering a lavage composition to a subject, for example, comprising 4 L of a polyethylene glycol/electrolyte solution (U.S. Patent Application Publication No. 20070298008, incorporated by reference in its entirety). Some methods of obtaining a gastrointestinal lavage fluid include antegrade lavage and retrograde lavage.

More methods of obtaining a gastrointestinal lavage fluid include oral administration of lavage compositions. Such lavage composition may include solutions of electrolytes, such as sodium, potassium and magnesium salts of sulfate, bicarbonate, chloride, phosphate or citrate. Some such compositions may also include polyethylene glycol, which can act as a non-absorbable osmotic agent. Generic compositions include polyethylene glycol with an electrolyte solution, optionally also including bisacodyl, or ascorbic acid, and compositions including sulfate salts such as sodium sulfate, magnesium sulfate, or potassium sulfate. In some embodiments, an oral lavage fluid can include magnesium citrate. In some embodiments, an oral lavage fluid can include sodium picosulfate. One example composition of an oral lavage solution comprising polyethylene glycol with an electrolyte solution is GOLYTELY (Braintree Labs. Inc.). GOLYTELY is formulated as follows: polyethylene glycol 59 g, sodium sulfate 5.68 g, sodium bicarbonate 1.69 g, sodium chloride 1.46 g, potassium chloride 0.745 g and water to make up one liter (Davis et al. (1980) Gastroenterology 78:991-995, incorporated by reference in its entirety). Ingestion of GOLYTELY produces a voluminous, liquid stool with minimal changes in the subject's water and electrolyte balance. Another example of an oral lavage composition comprising polyethylene glycol with an electrolyte solution is NULYTELY (Braintree Labs. Inc.). Another exemplary oral lavage composition is HALFLYTELY (Braintree Labs. Inc.) which includes polyethylene glycol with an electrolyte solution and bisacodyl. An exemplary oral lavage composition comprising sulfate salts, such as sodium sulfate, magnesium sulfate, or potassium sulfate is SUPREP (Braintree Labs. Inc.). An exemplary composition of an oral lavage solution comprising polyethylene glycol with an electrolyte solution and ascorbic acid is MOVIPREP (Salix Pharmaceuticals, Inc.).

Polyethylene glycol is effective as an oral lavage composition when large amounts of polyethylene glycol are administered in large volumes of a dilute salt solution. Usually about 250-400 g polyethylene glycol are administered to the subject in about 4 L of an electrolyte solution in water. Oral administration of polyethylene glycol can be used to produce a bowel movement over a period of time, e.g., overnight. The dose required will vary, but from about 10-100 g of polyethylene glycol in 8 oz. of water can be effective. A dose of from about 68-85 g of polyethylene glycol can be effective to produce an overnight bowel movement, without profuse diarrhea. A volume of a solution of polyethylene glycol in an isotonic fluid can be an effective amount of an osmotic laxative. Volumes from about 0.5 L to about 4 L can be effective. Preferably the effective volume is between about 1.5 L and about 2.5 L. Oral administration of 2 L of isotonic solution is effective.

More examples of oral lavage compositions include hypertonic solutions of non-phosphate salts with an osmotic laxative agent such as polyethylene glycol (U.S. Pat. App. No. 20090258090, incorporated by reference in its entirety). Mixtures of sulfate salts that omit phosphates, for example, effective amounts of one or more of the following sulfate salts Na₂SO₄, MgSO₄, and K₂SO₄ can be effective (e.g., SUPREP). Some embodiments include about 0.1 g to about 20.0 g Na₂SO₄, and from about 1.0 g to 10.0 g Na₂SO₄ may be useful. Dosage amounts of MgSO₄ from about 0.01 g to about 40.0 g can be effective. Doses of from about 0.1 g to about 20.0 g Na₂SO₄ may also be advantageously used, as well as dosages of 1.0 to 10.0 g. Dosage amounts of K₂SO₄ from about 0.01 g to about 20.0 g can be effective to produce purgation, and doses of from about 0.1 g to about 10.0 g and from about 0.5 g to about 5.0 g K₂SO₄ may also be useful. Addition of an osmotic laxative agent, such as polyethylene glycol (PEG) may improve the effectiveness of the above salt mixtures. Doses of PEG from about 1.0 g to about 100 g PEG are effective. Doses from about 10.0 g to about 50 g of PEG are also effective, as is a dose of about 34 g. For ease of administration, the above mixture of salts can be dissolved in a convenient volume of water. A volume of less than one liter of water can be well tolerated by most subjects. The mixture can be dissolved in any small volume of water, and volumes of between 100 and 500 ml are useful. The effective dose may be divided and administered to the patient in two or more administrations over an appropriate time period. Generally, administration of two doses of equal portions of the effective dose, separated by 6 to 24 hours, produces satisfactory purgation. Some embodiments include cessation of normal oral intake during a defined period before and during administration of an oral lavage composition.

Some lavage compositions include a laxative, such as bisacodyl. In some embodiments, a laxative can be co-administered to a subject with a lavage composition. As will be understood, such co-administration can include, for example, administration of a laxative up to several hours before administration of a lavage composition to a subject, administration of a laxative with the administration of a lavage composition to a subject, or administration of a laxative up to several hours after administration of a lavage composition to a subject. Examples of laxatives and their effective doses include Aloe, 250-1000 mg; Bisacodyl, about 5-80 mg; Casanthranol, 30-360 mg; Cascara aromatic fluid extract, 2-24 ml; Cascara sagrada bark, 300-4000 mg; Cascada sagrada extract, 300-2000 mg; Cascara sagrada fliuid extract, 0.5-5.0 ml; Castor oil, 15-240 ml; Danthron, 75-300 mg; Dehydrocholic Acid, 250-2000 mg; Phenolphthalein, 30-1000 mg; Sennosides A and B, 12-200 mg; and Picosulfate, 1-100 mg.

More examples of lavage compositions include aqueous solutions of concentrated phosphate salts. The aqueous phosphate salt concentrate produces an osmotic effect on the intra-luminal contents of the gastrointestinal tract. Evacuation of the bowel occurs with a large influx of water and electrolytes into the colon from the body. One exemplary composition comprises 480 g/L monobasic sodium phosphate and 180 g/L dibasic sodium phosphate in stabilized buffered aqueous solution (FLEET'S PHOSPHO-SODA, C. S. Fleet Co., Inc.). Subjects are typically required to take 2-3 oz doses of this composition, separated by a 3 to 12 hour interval for a total of 6 ounces (180 ml).

Gastrointestinal lavage fluid may be collected from a subject before, during, or after a medical or diagnostic procedure. In some embodiments, a subject may collect gastrointestinal lavage fluid, for example, using a receptacle such as a toilet insert which captures the fluid. Enzyme inhibitors and denaturants may be used to preserve the quality of the gastrointestinal lavage fluid. In some embodiments, the pH of the sample may be adjusted to help stabilize the samples. In some embodiments, gastrointestinal lavage fluid samples may be further treated to remove some or all solids and/or bacteria, such as by centrifugation or filtration. In some embodiments, the gastrointestinal tract may not be fully purged by administration of an oral lavage composition. For example, a portion of a complete dose of an oral lavage composition required to fully purge the gastrointestinal tract of a subject can be administered to the subject. In some embodiments, a gastrointestinal lavage fluid can comprise fecal matter. In more embodiments, fecal matter can comprise a gastrointestinal lavage fluid.

### Methods for Detecting Pancreatic Cancer Biomarkers

The level of pancreatic biomarker proteins in a sample obtained from a subject may be determined by any of a wide variety of techniques and methods, which transform the pancreatic biomarker proteins within the sample into a moiety that can be detected and quantified. Non-limiting examples of such methods include analyzing the sample using immunological methods for detection of proteins, protein purification methods, protein function or activity assays, nucleic acid hybridization methods, nucleic acid reverse transcription methods, and nucleic acid amplification methods, immunohistological, immunocytological, hybridization using immunofluorescence and/or immunoenzymatic, hydrometry, polarimetry, spectrophotometry (e.g., mass and NMR), chromatography (e.g., gas liquid, high performance liquid, and thin layer), immunoblotting, Western blotting, Northern blotting, electron microscopy, mass spectrometry, e.g., MALDI-TOF and SELDI-TOF, immunoprecipitations, immunofluorescence, immunohistochemistry, enzyme linked immunosorbent assays (ELISAs), e.g., amplified ELISA, quantitative blood based assays, e.g., serum ELISA, quantitative urine based assays, flow cytometry, Southern hybridizations, array analysis, and the like, and combinations or sub-combinations thereof. In some embodiments, nucleic acid encoding pancreatic cancer biomarker proteins may be detected using nucleic acid hybridization methods, such as Southern blotting, Northern blotting, or PCR.

Some embodiments of the methods and compositions provided herein include characterizing a pancreatic cancer biomarker in a sample, such as a sample obtained from the gastrointestinal tract, including a gastrointestinal lavage fluid and/or fecal sample. Characterizing a pancreatic cancer biomarker can include, for example, identifying a pancreatic cancer biomarker, detecting a pancreatic cancer biomarker, and/or quantifying a pancreatic cancer biomarker.

Some embodiments include identifying, determining the presence or absence of a pancreatic cancer biomarker, and/or quantifying a pancreatic cancer biomarker, wherein the pancreatic cancer biomarker comprises a peptide, polypeptide, protein and/or non-proteinaceous biological molecule.

As used in the present specification, the term “polypeptide” and “protein”, used interchangeably herein, refer to a polymer of amino acids without regard to the length of the polymer; thus, peptides, oligopeptides, and proteins are included within the definition of polypeptide. This term also includes wild-type polypeptides, as well as mutants, truncations, extensions, splice-variants, and other non-native forms of polypeptide that may be present. This term also includes forms of the foregoing that have been subject to enzymatic degradation by proteases or other mechanisms (enzymatic or non-enzymatic) in the subject. For example, a polypeptide may be subject to degradation by a protease to produce a polypeptide fragment of the polypeptide. The protease may be one that is expressed or increased in expression as a result of the health problem or disease of the gastrointestinal tract system. This term also does not specify or exclude chemical or post-expression/translational modifications of the polypeptides, although chemical or post-expression modifications of these polypeptides may be included or excluded as specific embodiments. Therefore, for example, modifications to polypeptides that include the covalent attachment of glycosyl groups (i.e., glycosylation), acetyl groups (i.e., acetylation), phosphate groups (phosphorylation, including, but not limited to, phosphorylation on serine, threonine and tyrosine groups), lipid groups and the like are expressly encompassed by the term polypeptide. Further, polypeptides with these modifications may be specified as individual species to be included or excluded. The natural or other chemical modifications, such as those listed in the examples above, can occur anywhere in a polypeptide, including the peptide backbone, the amino acid side-chains and the amino or carboxyl termini, and may be present in the same or varying degrees at several sites in a gastrointestinal tract polypeptide. Also, a gastrointestinal tract polypeptide may contain many types of modifications. Polypeptides may be branched, for example, as a result of ubiquitination, and they may be cyclic, with or without branching. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formylation of cysteine, formylation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination (see, for instance Creighton, (1993), Posttranslational Covalent Modification of Proteins, W. H. Freeman and Company, New York B. C. Johnson, Ed., Academic Press, New York 1-12; Seifier, et al., (1990) Meth Enzymol 182:626-646; Rattan et al, (1992) Ann NY Acad Sci 663:48-62). Isoforms of the proteins disclosed herein are also intended to be encompassed by the methods of the present invention.

Such pancreatic cancer biomarkers may be characterized by a variety of methods such as immunoassays, including radioimmunoassays, enzyme-linked immunoassays and two-antibody sandwich assays as described herein. A variety of immunoassay formats, including competitive and non-competitive immunoassay formats, antigen capture assays and two-antibody sandwich assays also are useful (Self and Cook, (1996) Curr. Opin. Biotechnol. 7:60-65, incorporated by reference in its entirety). Some embodiments include one or more antigen capture assays. In an antigen capture assay, antibody is bound to a solid phase, and sample is added such that antigen, e.g., a pancreatic cancer biomarker in a fluid or tissue sample, is bound by the antibody. After unbound proteins are removed by washing, the amount of bound antigen can be quantitated, if desired, using, for example, a radioassay (Harlow and Lane, (1988) Antibodies A Laboratory Manual Cold Spring Harbor Laboratory: New York, incorporated by reference in its entirety). Immunoassays can be performed under conditions of antibody excess, or as antigen competitions, to quantitate the amount of antigen and, thus, determine a level of a pancreatic cancer biomarker in a sample

Enzyme-linked immunosorbent assays (ELISAs) can be useful in certain embodiments provided herein. An enzyme such as horseradish peroxidase (HRP), alkaline phosphatase (AP), β-galactosidase or urease can be linked, for example, to an anti-HMGB1 antibody or to a secondary antibody for use in a method of the invention. A horseradish-peroxidase detection system can be used, for example, with the chromogenic substrate tetramethylbenzidine (TMB), which yields a soluble product in the presence of hydrogen peroxide that is detectable at 450 nm. Other convenient enzyme-linked systems include, for example, the alkaline phosphatase detection system, which can be used with the chromogenic substrate p-nitrophenyl phosphate to yield a soluble product readily detectable at 405 nm. Similarly, a β-galactosidase detection system can be used with the chromogenic substrate o-nitrophenyl-β-D-galactopyranoside (ONPG) to yield a soluble product detectable at 410 nm, or a urease detection system can be used with a substrate such as urea-bromocresol purple (Sigma Immunochemicals). Useful enzyme-linked primary and secondary antibodies can be obtained from a number of commercial sources such as Jackson Immuno-Research (West Grove, Pa.), as described further herein.

In certain embodiments, a pancreatic cancer biomarker in a sample, such as a sample obtained from the gastrointestinal tract, for example a gastrointestinal lavage fluid or fecal matter, can be detected and/or measured using chemiluminescent detection. For example in certain embodiments, specific antibodies to a particular pancreatic cancer biomarker are used to capture the pancreatic cancer biomarker present in the biological sample, e.g., such as a sample obtained from the gastrointestinal tract, for example, a gastrointestinal lavage fluid or fecal matter, and an antibody specific for the pancreatic cancer biomarker-specific antibodies and labeled with an chemiluminescent label is used to detect the pancreatic cancer biomarker present in the sample. Any chemiluminescent label and detection system can be used in the present methods. Chemiluminescent secondary antibodies can be obtained commercially from various sources such as Amersham. Methods of detecting chemiluminescent secondary antibodies are known in the art.

Fluorescent detection also can be useful for detecting a pancreatic cancer biomarker in certain methods provided herein. Useful fluorochromes include DAPI, fluorescein, Hoechst 33258, R-phycocyanin, B-phycoerythrin, R-phycoerythrin, rhodamine, Texas red and lissamine. Fluorescein or rhodamine labeled antibodies, or fluorescein- or rhodamine-labeled secondary antibodies.

Radioimmunoassays (RIAs) also can be useful in certain methods provided herein. Radioimmunoassays can be performed, for example, with ¹²⁵I-labeled primary or secondary antibody (Harlow and Lane, (1988) Antibodies A Laboratory Manual Cold Spring Harbor Laboratory: New York, incorporated by reference in its entirety).

A signal from a detectable reagent can be analyzed, for example, using a spectrophotometer to detect color from a chromogenic substrate; a radiation counter to detect radiation, such as a gamma counter for detection of ¹²⁵I; or a fluorometer to detect fluorescence in the presence of light of a certain wavelength. Where an enzyme-linked assay is used, quantitative analysis of the amount of a pancreatic cancer biomarker can be performed using a spectrophotometer such as an EMAX Microplate Reader (Molecular Devices; Menlo Park, Calif.) in accordance with the manufacturer's instructions. The assays of the invention can be automated or performed robotically, if desired, and that the signal from multiple samples can be detected simultaneously.

In some embodiments, capillary electrophoresis based immunoassays (CEIA), which can be automated if desired, may be used to detect and/or measure the pancreatic cancer biomarker. Immunoassays also can be used in conjunction with laser-induced fluorescence as described, for example, in Schmalzing and Nashabeh, Electrophoresis 18:2184-93 (1997), and Bao, J. Chromatogr. B. Biomed. Sci. 699:463-80 (1997), each incorporated by reference in its entirety. Liposome immunoassays, such as flow-injection liposome immunoassays and liposome immunosensors, also can be used to detect pancreatic cancer biomarkers or to determine a level of a pancreatic cancer biomarker according to certain methods provided herein (Rongen et al., (1997) J. Immunol. Methods 204:105-133, incorporated by reference in its entirety).

Sandwich enzyme immunoassays also can be useful in certain embodiments. In a two-antibody sandwich assay, a first antibody is bound to a solid support, and the antigen is allowed to bind to the first antibody. The amount of a pancreatic cancer biomarker is quantitated by measuring the amount of a second antibody that binds to it.

In an exemplary sandwich assay, an agent that selectively binds to a pancreatic cancer biomarker can be immobilized on a solid support. A capture reagent can be chosen to directly bind the pancreatic cancer biomarker or indirectly bind the pancreatic cancer biomarker by binding with an ancillary specific binding member which is bound to the pancreatic cancer biomarker. In addition, the capture reagent may be immobilized on the solid phase before or during the performance of the assay by means of any suitable attachment method. Typically, the capture site of the present invention is a delimited or defined portion of the solid phase such that the specific binding reaction of the capture reagent and analyte is localized or concentrated in a limited site, thereby facilitating the detection of label that is immobilized at the capture site in contrast to other portions of the solid phase. In a related embodiment, the capture reagent can be applied to the solid phase by dipping, inscribing with a pen, dispensing through a capillary tube, or through the use of reagent jet-printing or other techniques. In addition, the capture zone can be marked, for example, with a dye, such that the position of the capture zone upon the solid phase can be visually or instrumentally determined even when there is no label immobilized at the site.

Another exemplary embodiment of a sandwich assay format includes methods wherein a sample is mixed with a labeled first specific binding pair member for the pancreatic cancer biomarker and allowed to traverse a lateral flow matrix, past a series of spatially separated capture zones located on the matrix (See e.g., U.S. Pat. No. 7,491,551, incorporated by reference in its entirety). The sample may be mixed with the labeled first specific binding pair member prior to addition of the sample to the matrix. Alternatively, the labeled first specific binding pair member may be diffusively bound on the matrix on a labeling zone at a point upstream of the series of capture zones. Optionally, the sample is added directly to the labeling zone. Preferably, the sample is added to a sample receiving zone on the matrix at a point upstream of the labeling zone and allowed to flow through the labeling zone. The labeled first specific binding pair member located within the labeling zone is capable of being freely suspendible in the sample. Therefore, if analyte is present in the sample, the labeled first specific binding pair member will bind to the pancreatic cancer biomarker and the resulting pancreatic cancer biomarker-labeled first specific binding pair member complex will be transported to and through the capture zones. The extent of complex formation between the pancreatic cancer biomarker and the labeled specific binding pair member is directly proportional to the amount of pancreatic cancer biomarker present in the sample. A second specific binding pair member capable of binding to the pancreatic cancer biomarker-first specific binding pair member complex is immobilized on each of the capture zones. This second specific binding pair member is not capable of binding the labeled specific binding pair member unless the labeled specific binding pair member is bound to the pancreatic cancer biomarker. Thus, the amount of labeled specific binding pair member that accumulates on the capture zones is directly proportional to the amount of pancreatic cancer biomarker present in the sample.

In some embodiments, an assay includes the use of binding agent immobilized on a solid support to bind to and remove a target polypeptide from the remainder of the sample. The bound target polypeptide may then be detected using a detection reagent that contains a reporter group and specifically binds to the binding agent/polypeptide complex. Such detection reagents may comprise, for example, a binding agent that specifically binds to the target polypeptide or an antibody or other agent that specifically binds to the binding agent, such as an anti-immunoglobulin, protein G, protein A or a lectin. In such embodiments, the binding agent can comprise an antibody or antigen-binding fragment thereof specific to a polypeptide or fragment thereof descried herein. Alternatively, a competitive assay may be utilized in which a polypeptide is labeled with a reporter group and allowed to bind to the immobilized binding agent after incubation of the binding agent with the sample. The extent to which components of the sample inhibit the binding of the labeled polypeptide to the binding agent is indicative of the reactivity of the sample with the immobilized binding agent. Suitable polypeptides for use within such assays include full length proteins provided herein and polypeptide portions thereof such as SEQ ID NOs:1-793, for example, SEQ ID NOs:1-19, 47, 49-58, 206, 726, 729, 780 or 793, to which the binding agent binds.

The solid support may be any material known to those of ordinary skill in the art to which the binding agent may be attached. For example, the solid support may be a test well in a microtiter plate or a nitrocellulose or other suitable membrane or flow-through format or test strip. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681. The binding agent may be immobilized on the solid support using a variety of techniques known to those of skill in the art, which are amply described in the patent and scientific literature. In the context of the present invention, the term “immobilization” refers to both noncovalent association, such as adsorption, and covalent attachment (which may be a direct linkage between the agent and functional groups on the support or may be a linkage by way of a cross-linking agent). Immobilization by adsorption to a well in a microtiter plate or to a membrane is preferred. In such cases, adsorption may be achieved by contacting the binding agent, in a suitable buffer, with the solid support for a suitable amount of time. The contact time varies with temperature, but is typically between about 1 hour and about 1 day. In general, contacting a well of a plastic microtiter plate (such as polystyrene or polyvinylchloride) with an amount of binding agent ranging from about 10 ng to about 10 μg, and preferably about 100 ng to about 1 μg, is sufficient to immobilize an adequate amount of binding agent.

Covalent attachment of binding agent to a solid support may generally be achieved by first reacting the support with a bifunctional reagent that will react with both the support and a functional group, such as a hydroxyl or amino group, on the binding agent. For example, the binding agent may be covalently attached to supports having an appropriate polymer coating using benzoquinone or by condensation of an aldehyde group on the support with an amine and an active hydrogen on the binding partner (see, e.g., Pierce Immunotechnology Catalog and Handbook, 1991, at A12-A13).

In certain embodiments, the assay is a two-antibody sandwich assay. This assay may be performed by first contacting an antibody that has been immobilized on a solid support, commonly the well of a microtiter plate, with the sample, such that target polypeptides within the sample are allowed to bind to the immobilized antibody. Unbound sample is then removed from the immobilized polypeptide-antibody complexes and a detection reagent (preferably a second antibody capable of binding to a different site on the polypeptide) containing a reporter group is added. The amount of detection reagent that remains bound to the solid support is then determined using a method appropriate for the specific reporter group.

More specifically, once the antibody is immobilized on the support as described above, the remaining protein binding sites on the support are typically blocked. Any suitable blocking agent known to those of ordinary skill in the art may be used, such as bovine serum albumin or TWEEN 20. (Sigma Chemical Co., St. Louis, Mo.). The immobilized antibody is then incubated with the sample, and target polypeptide is allowed to bind to the antibody. The sample may be diluted with a suitable diluent, such as phosphate-buffered saline (PBS) prior to incubation. In general, an appropriate contact time (i.e., incubation time) is a period of time that is sufficient to detect the presence of target polypeptide within a sample obtained from an individual with breast cancer. Preferably, the contact time is sufficient to achieve a level of binding that is at least about 95% of that achieved at equilibrium between bound and unbound polypeptide. Those of ordinary skill in the art will recognize that the time necessary to achieve equilibrium may be readily determined by assaying the level of binding that occurs over a period of time. At room temperature, an incubation time of about 30 minutes is generally sufficient.

Unbound sample may then be removed by washing the solid support with an appropriate buffer, such as PBS containing 0.1% TWEEN 20. The second antibody, which contains a reporter group, may then be added to the solid support. Reporter groups are well known in the art. The detection reagent is then incubated with the immobilized antibody-polypeptide complex for an amount of time sufficient to detect the bound detection reagent. An appropriate amount of time may generally be determined by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

To determine the level of a polypeptide described herein e.g., SEQ ID NOs:1-793 and, in particular, SEQ ID NOs: 1-19, 47, 49-58, 206, 726, 729, 780 or 793, the signal detected from the reporter group that remains bound to the solid support is generally compared to a signal that corresponds to a predetermined cut-off value. In one embodiment, the cut-off value for the detection of a cancer is the average mean signal obtained when the immobilized antibody is incubated with samples from patients without the cancer. In general, a sample generating a signal that is three standard deviations above or below the predetermined cut-off value is considered positive for the cancer. For example, an increased level of certain polypeptides described herein e.g., SEQ ID NOs:17-19, 47, 726, 729 or 780, may be indicative of the presence of cancer or the stage of cancer, such as pancreatic cancer. Similarly, a reduced level of certain polypeptides described herein e.g., SEQ ID NOs:1-16, 49, 55-58, 206 or 793, may be indicative of the presence of cancer or the stage of cancer. In some embodiments, the cut-off value is determined using a Receiver Operator Curve, according to the method of Sackett et al., Clinical Epidemiology: A Basic Science for Clinical Medicine, Little Brown and Co., 1985, p. 106-7. Briefly, in this embodiment, the cut-off value may be determined from a plot of pairs of true positive rates (i.e., sensitivity) and false positive rates (100%-specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate.

In a related embodiment, the assay is performed in a flow-through or test strip format, wherein the binding agent is immobilized on a membrane, such as nitrocellulose. In the flow-through test, target polypeptides within the sample bind to the immobilized binding agent as the sample passes through the membrane. A second labeled binding agent then binds to the binding agent-polypeptide complex as a solution containing the second binding agent flows through the membrane. The detection of bound second binding agent may then be performed as described herein. In the test strip format, one end of the membrane to which binding agent is bound is immersed in a solution containing the sample. The sample migrates along the membrane through a region containing second binding agent and to the area of immobilized binding agent. The amount of immobilized antibody indicates the presence, or absence or progression or stage of a cancer. Typically, the concentration of second binding agent at that site generates a pattern, such as a line, that can be read visually. In general, the amount of binding agent immobilized on the membrane is selected to generate a visually discernible pattern when the biological sample contains a level of polypeptide that would be sufficient to generate a positive signal in the two-antibody sandwich assay, in the format discussed above. Preferred binding agents for use in such assays are antibodies and antigen-binding fragments thereof. Preferably, the amount of antibody immobilized on the membrane ranges from about 25 ng to about 1 μg, and more preferably from about 50 ng to about 500 ng. Such tests can typically be performed with a very small amount of biological sample.

Quantitative Western blotting also can be used to detect a pancreatic cancer biomarker or to determine a level of pancreatic cancer biomarker in a method provided herein. Western blots can be quantitated by well known methods such as scanning densitometry. As an example, protein samples are electrophoresed on 10% SDS-PAGE Laemmli gels. Primary murine monoclonal antibodies, for example, against a pancreatic cancer biomarker are reacted with the blot, and antibody binding confirmed to be linear using a preliminary slot blot experiment. Goat anti-mouse horseradish peroxidase-coupled antibodies (BioRad) are used as the secondary antibody, and signal detection performed using chemiluminescence, for example, with the Renaissance chemiluminescence kit (New England Nuclear; Boston, Mass.) according to the manufacturer's instructions. Autoradiographs of the blots are analyzed using a scanning densitometer (Molecular Dynamics; Sunnyvale, Calif.) and normalized to a positive control. Values are reported, for example, as a ratio between the actual value to the positive control (densitometric index). Such methods are described, for example, in Parra et al., J. Vasc. Surg. 28:669-675 (1998), incorporated herein by reference in its entirety.

As described herein, immunoassays including, for example, enzyme-linked immunosorbent assays, radioimmunoassays and quantitative western analysis, can be useful in some embodiments for detecting a pancreatic cancer biomarker or determining a level of a pancreatic cancer biomarker. Such assays typically rely on one or more antibodies. As would be understood by the skilled artisan, methods described herein can be used to readily distinguish proteins with alternative forms of post-translation modifications, e.g., phosphorylated proteins, and glycosylated proteins.

Some embodiments of the methods and compositions provided herein include generating agents that selectively bind to pancreatic cancer biomarkers. In some embodiments, such agents include an antibody or antigen-binding fragment thereof. Methods of generating polyclonal antibodies and monoclonal antibodies are well known in the art. The antibodies or active fragments thereof may be obtained by methods known in the art for production of antibodies or functional portions thereof. Such methods include, but are not limited to, separating B cells with cell-surface antibodies of the desired specificity, cloning the DNA expressing the variable regions of the light and heavy chains and expressing the recombinant genes in a suitable host cell. Standard monoclonal antibody generation techniques can be used wherein the antibodies are obtained from immortalized antibody-producing hybridoma cells. These hybridomas can be produced by immunizing animals with HSCs or progeny thereof, and fusing B lymphocytes from the immunized animals, preferably isolated from the immunized host spleen, with compatible immortalized cells, preferably a B cell myeloma.

In embodiments where the pancreatic cancer biomarker is a polypeptide associated with one or more iron atoms, antibodies which differentially bind to the iron-associated polypeptide relative to the same polypeptide without iron can be prepared. Antibodies which differentially bind to metal-associated polypeptides relative to the same polypeptide without metal and methods for making such antibodies have been described, for example, in HALLAB, et al., In vitro Reactivity to Implant Metals Demonstrates a Person Dependent Association with both T-Cell and B-Cell Activation, J. Biomed Mater Res A, 2010 February; 92(2):667-682; KONG, et al., Preparation of specific monoclonal antibodies against chelated copper ions, Biol Trace Elem Res., 2012 March; 145(3):388-395; LIU, et al., Preparation and characterization of monoclonal antibody specific for copper-chelate complex, J Immunol Methods., 2013 Jan. 31; 387(1-2):228-236; XIANG, et al., A competitive indirect enzyme-linked immunoassay for lead ion measurement using mAbs against the lead-DTPA complex, Environ Pollut., 2010 May; 158(5):1376-1380; YANG, et al., Detection of antibodies against corrosion products in patients after Co—Cr total joint replacements, J Biomed Mater Res., 1994 November; 28(11):1249-1258; ZHANG, et al., Development of ELISA for detection of mercury based on specific monoclonal antibodies against mercury-chelate, Biol Trace Elem Res., 2011 December; 144(1-3):854-864; and ZHU, et al., Preparation of specific monoclonal antibodies (MAbs) against heavy metals: MAbs that recognize chelated cadmium ions, J Agric Food Chem., 2007 Sep. 19; 55(19):7648-7653, each of which is incorporated by reference in its entirety.

Pancreatic cancer biomarkers, such as protein pancreatic cancer biomarkers, can be characterized, isolated, purified, or obtained for use in generating antibodies by a variety of methods. Proteins, polypeptides and peptides can be isolated by a variety of methods well known in the art, such as protein precipitation, chromatography (e.g., reverse phase chromatography, size exclusion chromatography, ion exchange chromatography, liquid chromatography), affinity capture, and differential extractions.

Isolated proteins can undergo enzymatic digestion or chemical cleavage to yield polypeptide fragments and peptides. Such fragments can be identified and quantified. A particularly useful method for analysis of polypeptide/peptide fragments and other pancreatic cancer biomarkers is mass spectrometry (U.S. Pat. App. No. 20100279382, incorporated by reference in its entirety). A number of mass spectrometry-based quantitative proteomics methods have been developed that identify the proteins contained in each sample and determine the relative abundance of each identified protein across samples (Flory et al., Trends Biotechnol. 20:S23-29 (2002); Aebersold, J. Am. Soc. Mass Spectrom. 14:685-695 (2003); Aebersold, J. Infect. Dis. 187 Suppl 2:S315-320 (2003); Patterson and Aebersold, Nat. Genet. 33 Suppl, 311-323 (2003); Aebersold and Mann, Nature 422:198-207 (2003); Aebersold, R. and Cravatt, Trends Biotechnol. 20:S1-2 (2002); Aebersold and Goodlett, Chem. Rev. 101, 269-295 (2001); Tao and Aebersold, Curr. Opin. Biotechnol. 14:110-118 (2003), each incorporated by reference in its entirety). Generally, the proteins in each sample are labeled to acquire an isotopic signature that identifies their sample of origin and provides the basis for accurate mass spectrometric quantification. Samples with different isotopic signatures are then combined and analyzed, typically by multidimensional chromatography tandem mass spectrometry. The resulting collision induced dissociation (CID) spectra are then assigned to peptide sequences and the relative abundance of each detected protein in each sample is calculated based on the relative signal intensities for the differentially isotopically labeled peptides of identical sequence.

More techniques for identifying and quantifying pancreatic cancer biomarkers include label-free quantitative proteomics methods. Such methods include: (i) sample preparation including protein extraction, reduction, alkylation, and digestion; (ii) sample separation by liquid chromatography (LC or LC/LC) and analysis by MS/MS; (iii) data analysis including peptide/protein identification, quantification, and statistical analysis. Each sample can be separately prepared, then subjected to individual LC-MS/MS or LC/LC-MS/MS runs (Zhu W. et al., J. of Biomedicine and Biotech. (2010) Article ID 840518, 6 pages, incorporated by reference in its entirety). An exemplary technique includes LC-MS in which the mass of a peptide coupled with its corresponding chromatographic elution time as peptide properties that uniquely define a peptide sequence, a method termed the accurate mass and time (AMT) tag approach. Using LC coupled with Fourier transform ion cyclotron resonance (LC-FTICR) MS to obtain the chromatographic and high mass accuracy information, peptide sequences can be identified by matching the AMT tags to previously acquired LC-MS/MS sequence information stored in a database. By taking advantage of the observed linear correlation between peak area of measured peptides and their abundance, these peptides can be relatively quantified by the signal intensity ratio of their corresponding peaks compared between MS runs (Tang, K., et al., (2004) J. Am. Soc. Mass Spectrom. 15:1416-1423; and Chelius, D. and Bondarenko, P. V. (2002) J. Proteome Res. 1: 317-323, incorporated by reference in their entireties). Statistics tools such as the Student's t-test can be used to analyse data from multiple LC-MS runs for each sample (Wiener, M. C., et al., (2004) Anal. Chem. 76:6085-6096, incorporated by reference in its entirety). At each point of acquisition time and m/z, the amplitudes of signal intensities from multiple LC-MS runs can be compared between two samples to detect peptides with statistically significant differences in abundance between samples.

As will be understood, a variety of mass spectrometry systems can be employed in the methods for identifying and/or quantifying a polypeptide/peptide fragments. Mass analyzers with high mass accuracy, high sensitivity and high resolution include ion trap, triple quadrupole, and time-of-flight, quadrupole time-of-flight mass spectrometeres and Fourier transform ion cyclotron mass analyzers (FT-ICR-MS). Mass spectrometers are typically equipped with matrix-assisted laser desorption (MALDI) or electrospray ionization (ESI) ion sources, although other methods of peptide ionization can also be used. In ion trap MS, analytes are ionized by ESI or MALDI and then put into an ion trap. Trapped ions can then be separately analyzed by MS upon selective release from the ion trap. Fragments can also be generated in the ion trap and analyzed. Sample molecules such as released polypeptide/peptide fragments can be analyzed, for example, by single stage mass spectrometry with a MALDI-TOF or ESI-TOF system. Methods of mass spectrometry analysis are well known to those skilled in the art (see, e.g., Yates, J. (1998) Mass Spect. 33:1-19; Kinter and Sherman, (2000) Protein Sequencing and Identification Using Tandem Mass. Spectrometry, John Wiley & Sons, New York; and Aebersold and Goodlett, (2001) Chem. Rev. 101:269-295, each incorporated by reference in its entirety).

For high resolution polypeptide fragment separation, liquid chromatography ESI-MS/MS or automated LC-MS/MS, which utilizes capillary reverse phase chromatography as the separation method, can be used (Yates et al., Methods Mol. Biol. 112:553-569 (1999), incorporated by reference in its entirety). Data dependent collision-induced dissociation (CID) with dynamic exclusion can also be used as the mass spectrometric method (Goodlett, et al., Anal. Chem. 72:1112-1118 (2000), incorporated by reference in its entirety).

Once a peptide is analyzed by MS/MS, the resulting CID spectrum can be compared to databases for the determination of the identity of the isolated peptide. Methods for protein identification using single peptides have been described previously (Aebersold and Goodlett, Chem. Rev. 101:269-295 (2001); Yates, J. Mass Spec. 33:1-19 (1998), David N. et al., Electrophoresis, 20 3551-67 (1999), each incorporated by reference in its entirety). In particular, it is possible that one or a few peptide fragments can be used to identify a parent polypeptide from which the fragments were derived if the peptides provide a unique signature for the parent polypeptide. Moreover, identification of a single peptide, alone or in combination with knowledge of a site of glycosylation, can be used to identify a parent glycopolypeptide from which the glycopeptide fragments were derived. As will be understood, methods that include MS can be used to characterize proteins, fragments thereof, as well as other types of pancreatic cancer biomarkers described herein.

In some embodiments, pancreatic cancer biomarkers include nucleic acids. Nucleic acids can encode a polypeptide or fragment thereof useful to determine the presence or absence of a cancer. As such, pancreatic cancer biomarkers include nucleic acid molecules sufficient for use as hybridization probes to identify nucleic acid molecules that correspond to a pancreatic cancer biomarker, including nucleic acids which encode a polypeptide corresponding to a pancreatic cancer biomarkers, and fragments of such nucleic acid molecules, e.g., those suitable for use as PCR primers for the amplification or mutation of nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

A nucleic acid pancreatic cancer biomarker can be amplified using cDNA, mRNA, or genomic DNA as a template and appropriate oligonucleotide primers according to standard PCR amplification techniques. The nucleic acid so amplified can be cloned into an appropriate vector and characterized by DNA sequence analysis. Furthermore, oligonucleotides corresponding to all or a portion of a nucleic acid pancreatic cancer biomarker can be prepared by standard synthetic techniques, e.g., using an automated DNA synthesizer.

In another preferred embodiment, a nucleic acid pancreatic cancer biomarker comprises a nucleic acid molecule that has a nucleotide sequence complementary to a nucleic acid which is differentially expressed in cancer or a fragment thereof. For example, the pancreatic cancer biomarker may comprise a nucleic acid encoding a polypeptide of any one of SEQ ID NOs:1-31 or 39-793, for example, SEQ ID NOs:1-19, 47, 49-58, 206, 726, 729, 780 or 793, or a fragment comprising at least 10, at least 20, at least 30, at least 40, at least 50 or more consecutive nucleotides thereof. A nucleic acid molecule which is complementary to a pancreatic cancer biomarker nucleotide sequence is one which is sufficiently complementary to the pancreatic cancer biomarker nucleotide sequence that it can hybridize to the pancreatic cancer biomarker nucleotide sequence thereby forming a stable duplex.

In some embodiments, a fragment of a polynucleotide sequence will be understood to include any nucleotide fragment having, for example, at least about 5 successive nucleotides, at least about 12 successive nucleotides, at least about 15 successive nucleotides, at least about 18 successive nucleotides, or at least about 20 successive nucleotides of the sequence from which it is derived. An upper limit for a fragment can include, for example, the total number of nucleotides in a full-length sequence encoding a particular polypeptide. A fragment of a polypeptide sequence will be understood to include any polypeptide fragment having, for example, at least about 5 successive residues, at least about 12 successive residues, at least about 15 successive residues, at least about 18 successive residues, or at least about 20 successive residues of the sequence from which it is derived. An upper limit for a fragment can include, for example, the total number of residues in a full-length sequence of a particular polypeptide.

Moreover, a nucleic acid pancreatic cancer biomarker can comprise all or only a portion of a nucleic acid sequence which is differentially expressed in cancer. For example, the pancreatic cancer biomarker may comprise a nucleic acid encoding a polypeptide of SEQ ID NOs:1-31 or 39-793, for example, SEQ ID NOs:1-19, 47, 49-58, 206, 726, 729, 780 or 793, or a fragment comprising at least 10, at least 20, at least 30, at least 40, at least 50 or more consecutive nucleotides thereof. Such nucleic acids can be used, for example, as a probe or primer. The probe/primer typically is used as one or more substantially purified oligonucleotides. The oligonucleotide typically comprises a region of nucleotide sequence that hybridizes under stringent conditions to at least about 7, preferably about 15, more preferably about 25, 50, 75, 100, 125, 150, 175, 200, 250, 300, 350, or 400 or more consecutive nucleotides of a nucleic acid.

Probes based on the sequence of a nucleic acid pancreatic cancer biomarker can be used to detect transcripts or genomic sequences corresponding to one or more pancreatic cancer biomarkers. The probe comprises a label group attached thereto, e.g., a radioisotope, a fluorescent compound, an enzyme, or an enzyme co-factor. Such probes can be used as part of a diagnostic test kit for identifying a biological sample, such as fluids, cells or tissues, which mis-express the protein, such as by measuring levels of a nucleic acid molecule encoding the protein in a sample of a fluid or cells from a subject, e.g., detecting mRNA levels or determining whether a gene encoding the protein has been mutated or deleted. Embodiments also include nucleic acid pancreatic cancer biomarkers that differ, due to degeneracy of the genetic code, from the nucleotide sequence of nucleic acids encoding a protein that corresponds to a pancreatic cancer biomarker, and thus encode the same protein.

### Method for Assessing the Presence, Absence or Progression of Pancreatic Cancer

Some of the methods and composition provided herein include methods for assessing the presence absence, progression or stage of a cancer, in particular pancreatic cancer, in a subject. Some such embodiments include determining the level of at least one pancreatic cancer biomarker in a sample from said subject. In some embodiments, the pancreatic cancer biomarker comprises at least one polypeptide or fragment thereof or at least one nucleic acid encoding the polypeptide. In some embodiments, the polypeptide is selected from any polypeptide provided herein, for example, SEQ ID NOs:1-31 or 39-793, for example, SEQ ID NOs:1-19, 47, 49-58, 206, 726, 729, 780 or 793.

In some embodiments, a sample is obtained from the gastrointestinal tract of a subject using methods provided herein.

Some embodiments include determining the level in the sample of at least 2 pancreatic cancer biomarkers, at least 3 pancreatic cancer biomarkers, at least 4 pancreatic cancer biomarkers, at least 5 pancreatic cancer biomarkers, at least 6 pancreatic cancer biomarkers, at least 7 pancreatic cancer biomarkers, at least 8 pancreatic cancer biomarkers, at least 9 pancreatic cancer biomarkers, at least 10 pancreatic cancer biomarkers, at least 11 pancreatic cancer biomarkers, at least 12 pancreatic cancer biomarkers, at least 13 pancreatic cancer biomarkers, at least 14 pancreatic cancer biomarkers, at least 15 pancreatic cancer biomarkers, at least 16 pancreatic cancer biomarkers, at least 17 pancreatic cancer biomarkers, at least 18 pancreatic cancer biomarkers, at least 19 pancreatic cancer biomarkers, or at least 20 pancreatic cancer biomarkers.

Some embodiments also include comparing the level of at least one pancreatic cancer biomarker in a sample of a subject with the level of the pancreatic cancer biomarker in a sample from a subject without the cancer. Some embodiments also include comparing the level of at least one pancreatic cancer biomarker in a sample of a subject with the level of the pancreatic cancer biomarker in a sample from a subject with the cancer.

Some embodiments also include comparing the level of at least one pancreatic cancer biomarker in a sample of a subject with the level of a control molecule. In some embodiments, the levels of a control molecule are determined in the sample from a subject. In some embodiments a control molecule comprises a non-pancreatic polypeptide. In some embodiments a control molecule comprises a non-pancreatic polypeptide that originates from the gastrointestinal tract. In some embodiments the levels of a control molecule are determined in the sample from a subject with cancer. In some embodiments the levels of a control molecule are determined in the sample from a subject without cancer. In some embodiments, the level of at least 1 control molecule is determined in a sample. In some embodiments, the level of at least about 2, 5, 10, or 15 control molecules are determined in a sample. Examples of control molecules include polypeptides and fragments thereof and nucleic acids encoding such polypeptides and fragments thereof, in which the polypeptide comprises, consists essentially of, or consists of SEQ ID NO:27, 32-40, 45, 54, 59 and 59. More examples of control molecules include CEA, and CA19-19.

In some embodiments, an increase in the level of the pancreatic cancer biomarker in a sample from a subject compared to the level of the pancreatic cancer biomarker in a sample from said subject without the cancer is indicative of the presence of the cancer in the subject. In some such embodiments, the pancreatic cancer biomarker can include a polypeptide or a fragment thereof, a nucleic acid encoding the polypeptide or fragment thereof, in which the polypeptide includes SEQ ID NOs: 17-19, 47, 726, 729 or 780.

In some embodiments, an increase in the level of a pancreatic cancer biomarker in a sample compared to the level of the pancreatic cancer biomarker in a sample obtained from a subject without a cancer is indicative of the cancer, in which the increase is at least about a 3-fold increase at least about a 5-fold increase, at least about a 10-fold increase, at least about a 20-fold increase, at least about a 30-fold increase, at least about a 40-fold increase, at least about a 50-fold increase, at least about a 60-fold increase, at least about a 70-fold increase, at least about a 80-fold increase, at least about a 90-fold increase, and at least about a 100-fold increase.

In some embodiments, a decrease in the level of the pancreatic cancer biomarker in a sample from a subject compared to the level of the pancreatic cancer biomarker in a sample from said subject without the cancer is indicative of the presence of the cancer in the subject. In some such embodiments, the pancreatic cancer biomarker can include a polypeptide or a fragment thereof, a nucleic acid encoding the polypeptide or fragment thereof, in which the polypeptide includes SEQ ID NOs:1-16, 49, 55-58, 206 or 793.

In some embodiments, a decrease in the level of a pancreatic cancer biomarker in a sample compared to the level of the pancreatic cancer biomarker in a sample obtained from a subject without a cancer is indicative of the cancer, in which the decrease is at least about a 3-fold decrease at least about a 5-fold decrease, at least about a 10-fold decrease, at least about a 20-fold decrease, at least about a 30-fold decrease, at least about a 40-fold decrease, at least about a 50-fold decrease, at least about a 60-fold decrease, at least about a 70-fold decrease, at least about a 80-fold decrease, at least about a 90-fold decrease, and at least about a 100-fold decrease.

Methods to determine the level of a pancreatic cancer biomarker in a sample are provided herein. In some embodiments, a method for determining the level of a pancreatic cancer biomarker, such as a polypeptide or fragment thereof, can include an immunoassay. Examples of an immunoassay include a Western blot, an enzyme linked immunoabsorbent assay (ELISA), and radioimmunoassay. In some embodiments, a method for determining the level of a pancreatic cancer biomarker, such as a polypeptide or fragment thereof, can include mass spectrometry.

### Kits

The present invention further provides a kit for determining the presence, absence, progression, or stage of a cancer in a subject comprising: (a) a lavage fluid for oral administration to a subject; (b) a vessel for collecting the gastrointestinal lavage fluid from the subject; and (c) an agent that selectively binds to at least one polypeptide or fragment thereof or nucleic acid encoding said polypeptide or fragment thereof, wherein said polypeptide comprises an amino acid sequence selected from the group consisting of a polypeptide comprising, consisting essentially of, or consisting of SEQ ID NOs:1-31 or 39-793, for example, SEQ ID NOs:1-19, 47, 49-58, 206, 726, 729, 780 or 793. Such kits can include at least 2, 3, 4, 5, 6, 7, 8, 9, or 10 agents that each selectively bind to a different polypeptide or a nucleic acid encoding said polypeptide or fragment thereof. In some embodiments, the agent comprises an antibody or antigen-binding fragment thereof.

Some embodiments of the methods and compositions provided herein include a kit comprising an agent which selectively binds to at least one polypeptide comprising an amino acid sequence selected from the group consisting of a polypeptide comprising, consisting essentially of, or consisting of SEQ ID NOs:1-31 or 39-793, for example, SEQ ID NOs:1-19, 47, 49-58, 206, 726, 729, 780 or 793, or a fragment thereof, wherein said agent is attached to a solid support. In some embodiments, the kit can include an agent that selectively binds to at least one polypeptide or nucleic acid encoding a polypeptide, wherein said polypeptide is selected from the group consisting of a polypeptide comprising, consisting essentially of, or consisting of SEQ ID NOs:1-16, 49, 55-58, 206 and 793. In some embodiments, the kit can include an agent that selectively binds to at least one polypeptide or nucleic acid encoding a polypeptide, wherein said polypeptide is selected from the group consisting of a polypeptide comprising, consisting essentially of, or consisting of SEQ ID NOs:17-19, 47, 726, 729 or 780. In some embodiments, the kit can include a plurality of agents that bind to different polypeptides comprising an amino acid sequence selected from the group consisting of a polypeptide comprising, consisting essentially of, or consisting of SEQ ID NOs:1-31 or 39-793, for example, SEQ ID NOs:1-19, 47, 49-58, 206, 726, 729, 780 or 793, or a fragment thereof are attached to said solid support. In some embodiments, the solid support comprises a solid phase test strip or a flow-through test strip. In some embodiments, the kit can also include a detectable agent which selectively binds to said polypeptide.

Some embodiments of the methods and compositions provided herein include a kit comprising an agent which selectively binds to at least one nucleic acid encoding a polypeptide comprising an amino acid sequence selected from the group consisting of a polypeptide comprising, consisting essentially of, or consisting of SEQ ID NOs:1-31 or 39-793, for example, SEQ ID NOs:1-19, 47, 49-58, 206, 726, 729, 780 or 793, or a fragment thereof, wherein said agent is attached to a solid support. In some embodiments, the kit can include an agent that selectively binds to at least one polypeptide or nucleic acid encoding a polypeptide, wherein said polypeptide is selected from the group consisting of a polypeptide comprising, consisting essentially of, or consisting of SEQ ID NOs:1-16, 49, 55-58, 206 and 793. In some embodiments, the kit can include an agent that selectively binds to at least one polypeptide or nucleic acid encoding a polypeptide, wherein said polypeptide is selected from the group consisting of a polypeptide comprising, consisting essentially of, or consisting of SEQ ID NOs:17-19, 47, 726, 729 or 780. In some embodiments, the kit can include a plurality of agents that bind to nucleic acids encoding different polypeptides comprising an amino acid sequence selected from the group consisting of a polypeptide comprising, consisting essentially of, or consisting of SEQ ID NOs:1-31 or 39-793, for example, SEQ ID NOs:1-19, 47, 49-58, 206, 726, 729, 780 or 793, or a fragment thereof are attached to said solid support. In some embodiments, the solid support comprises a solid phase test strip or a flow-through test strip. In some embodiments, the kit can also include a detectable agent which selectively binds to said polypeptide.

The present invention is further illustrated by the following examples which should not be construed as limiting in any way. The contents of all cited references, including literature references, issued patents and published patent applications, as cited throughout this application are hereby expressly incorporated herein by reference. It should further be understood that the contents of all the figures and tables attached hereto are expressly incorporated herein by reference.

### EXAMPLES

### Example 1

### Identification of Biomarkers Associated with Pancreatic Cancer

Gastrointestinal lavage fluid was obtained from patients with pancreatic cancer and from control patients, after administration of magnesium citrate (MgC) to the patients. Polypeptides were identified in gastrointestinal lavage fluid using mass spectrometry, and further characterized with MASCOT analysis. The presence or absence and/or levels of particular polypeptides were further confirmed using ELISA analyses. In the MASCOT analysis, a score indicates the relative prevalence of a protein or polypeptide, for example, a higher score indicates a greater prevalence for a particular protein or polypeptide in a sample, such that the most prevalent protein or polypeptide in sample will have the highest MASCOT score, and a ranking of “1.” Higher Mascot scores indicate better protein hits and can be correlated to relative protein levels. A score threshold of “>40” was indicative of a p-value significance of <0.05 as determined by the Mascot scoring system based on the search of this database with no enzyme specificity; a score of 40 is consistent with a p<0.01. Standard Mascot scoring was used whereby only the highest score was added for each peptide detected, even if it was sampled during MS/MS multiple times. For all data included, scores were all >40 in at least one sample per protein line. For additional confidence, the numbers of significant peptides were also reported and a minimum criteria of at least 2 peptides was selected. Very few had less than 3 peptides. All significant peptides counted represented different sequences (individual peptides) from their respective proteins. The score and numbers of significant peptides are reported in the format x/y where x is the score and y the number of significant peptides. If a protein was not detected in a particular sample it is listed as “ND”.

Gastrointestinal lavage fluid was collected from patients and analyzed with mass spectrometry (MS) and commercial ELISA. MS Data were acquired on an LTQ-Orbitrap mass spectrometer using input from an LC system. The A solvent contained 3% of B and 0.2% formic acid in water. The B solvent contained 3% of A and 0.2% formic acid in acetonitrile. Solvents were HPLC grade from Fisher. For a 120 min run, the starting solvent was 5% B and remains for 7 min. The gradient was changed to 10% by 13 min, 40% by 83 min, 90% by 103 min, then reduced from 90% to 5% at 111 min. It was then re-equilibrated for the next injection. Three injections were performed for each sample for repeatability determination. The MS was scanned (Orbitrap) over the mass range from 400 m/z to 2000 m/z every second while the LTQ (Trap) acquired up to 5 MSMS (peptide sequence) spectra in parallel. Data were acquired using the standard Thermo Xcalibur software. Peptides were eluted from a C18 LC column using triplicate injections. A search file was created from the triplicate injections from each lavage preparation (patient sample) and converted into a MGF (Mascot Generic Format) file using a combination of Xcalibur and Mascot software packages. Database searching was done using the Mascot search engine (Matrix Science, UK) against the RefSeq database (http://www.ncbi.nlm.nih.gov/RefSeq/) with taxonomy specified as human (homo sapiens), a mass accuracy of 10 ppm for the parent ion (MS) and 0.6 Da for the fragment ions (MS/MS), and “semitrypsin” selected. The RefSeq database was supplemented by the addition of antibody sequences that are included in the SwissProt protein database, as these antibody sequences are not part of the standard RefSeq listing.

Table 1 provides examples of proteins and polypeptides whose levels were found to have been reduced in pancreatic cancer. In Table 1, the proteins include pancreatic enzymes, such as lipase and amylase, and other pancreatic proteins such as lithostathine. The most dramatic change was observed with pancreatic triacylglycerol lipase precursor which was the most abundant protein in gastrointestinal lavage fluid from control patient, but was not detected (ND) in gastrointestinal lavage fluid from patient with pancreatic cancer.

TABLE 1
Proteins with Reduced Levels in Gastrointestinal
Lavage Fluid of Subjects with Pancreatic Cancer

| | NCBI | | | | MgC** Pancreatic | |
|---|---|---|---|---|---|---|
| SEQ ID | Accession | | MgC** Control | | Cancer | |
| NO.: | Nos. | Protein name | Ranking | Score | Ranking | Score |
| 1 | 10835000 | pancreatic triacylglycerol | 1 | 5010 | — | ND* |
| | | lipase precursor | | | | |
| 2 | 4502085 | pancreatic alpha-amylase | 2 | 4818 | 13 | 1947 |
| | | precursor | | | | |
| 3 | 10280622 | alpha-amylase 2B precursor | 3 | 4581 | 14 | 1933 |
| 4 | 4502997 | carboxypeptidase A1 | 4 | 3974 | 217 | 479 |
| | | precursor | | | | |
| 5 | 40254482 | alpha-amylase 1 precursor | 5 | 3675 | 18 | 1883 |
| 6 | 54607080 | carboxypeptidase B | 10 | 2567 | — | ND |
| | | preproprotein | | | | |
| 7 | 217416390 | carboxypeptidase A2 | 11 | 2504 | — | ND |
| | | precursor | | | | |
| 8 | 236460050 | chymotrypsin-like elastase | 17 | 1854 | 168 | 534 |
| | | family member 3A | | | | |
| | | preproprotein | | | | |
| 9 | 62526043 | chymotrypsin-C | 19 | 1649 | — | ND |
| | | preproprotein | | | | |
| 10 | 15559207 | chymotrypsin-like elastase | 21 | 1437 | 239 | 462 |
| | | family member 2A | | | | |
| | | preproprotein | | | | |
| 11 | 6679625 | chymotrypsin-like elastase | 24 | 1276 | 65 | 747 |
| | | family member 3B | | | | |
| | | preproprotein | | | | |
| 12 | 4506147 | trypsin-2 preproprotein | 26 | 1166 | 59 | 785 |
| 13 | 4506145 | trypsin-1 preproprotein | 32 | 1022 | 46 | 890 |
| 14 | 29725633 | lithostathine-1-alpha | 36 | 895 | 103 | 640 |
| | | precursor | | | | |
| 15 | 118498350 | chymotrypsinogen B2 | 42 | 770 | — | ND |
| | | precursor | | | | |
| 16 | 10835248 | lithostathine-1-beta precursor | 47 | 542 | — | ND |

*ND = not detected
**MgC = magnesium citrate

Table 2 provides examples of proteins and polypeptides whose levels were found to have increased in pancreatic cancer, with the most significant changes being for mucin-2.

TABLE 2
Proteins with Increased Levels in Gastrointestinal
Lavage Fluid of Subjects with Pancreatic Cancer

| | NCBI | | | | MgC** Pancreatic | |
|---|---|---|---|---|---|---|
| SEQ ID | Accession | | MgC** Control | | Cancer | |
| NO.: | Nos. | Protein name | Ranking | Score | Ranking | Score |
| 17 | 16306550 | selenium-binding | 25 | 1178 | 17 | 1887 |
| | | protein 1 | | | | |
| 18 | 83367077 | mucin-16 | 53 | 502 | 39 | 1018 |
| 19 | 116284392 | mucin-2 | 215 | 223 | 16 | 1921 |
| | | precursor | | | | |

**MgC = magnesium citrate

Table 3 provides examples of blood/serum proteins identified in gastrointestinal lavage fluid obtained from patients. Generally, blood proteins were found to have a low abundance in gastrointestinal lavage fluid obtained from patients. However, albumin was found to have increased levels in gastrointestinal lavage fluid obtained from patients with pancreatic cancer.

TABLE 3
Blood/Serum Proteins Present in Gastrointestinal
Lavage Fluid of Subjects with Pancreatic Cancer

| | NCBI | | Pancreatic | | | |
|---|---|---|---|---|---|---|
| SEQ ID | Accession | | Cancer | | Control | |
| NO.: | Nos. | Protein name | Ranking | Score | Ranking | Score |
| 20 | 4502027 | serum albumin | 1 | 4069 | 20 | 1638 |
| | | preproprotein | | | | |
| 21 | 4557871 | serotransferrin | 11 | 1252 | 287 | 199 |
| | | precursor | | | | |
| 22 | 115298678 | complement C3 | 117 | 247 | 779 | 130 |
| | | precursor | | | | |
| 23 | 50363217 | alpha-1-antitrypsin | 9 | 1531 | 16 | 1940 |
| | | precursor | | | | |
| 24 | 66932947 | alpha-2- | 60 | 452 | 336 | 190 |
| | | macroglobulin | | | | |
| | | precursor | | | | |
| 25 | 4557321 | apolipoprotein A-I | — | — | — | — |
| | | preproprotein | | | | |
| 26 | 324021745 | vitamin D-binding | 420 | 66 | 0 | 0 |
| | | protein isoform 3 | | | | |
| | | precursor | | | | |
| 27 | 105990532 | apolipoprotein B-100 | 222 | 118 | 86 | 351 |
| | | precursor | | | | |
| 28 | 4826762 | haptoglobin isoform 1 | 27 | 795 | 0 | 0 |
| | | preproprotein | | | | |
| 29 | 62739186 | complement factor H | 1827 | 29 | 0 | 0 |
| | | isoform a precursor | | | | |
| 30 | 4557485 | ceruloplasmin | 115 | 250 | 2820 | 40 |
| | | precursor | | | | |
| 31 | 11321561 | hemopexin precursor | 113 | 256 | 1594 | 83 |

Because the levels of certain proteins and polypeptides may vary between different samples, for example, between different patients, and between different samples taken from the same patient at different times, control proteins and polypeptides were identified in gastrointestinal lavage fluid from patients. Table 4 provides example proteins and polypeptides whose levels did not fluctuate significantly between patients with and without pancreatic cancer. The proteins and polypeptides listed in Table 4 include those that originate from the intestine. Some of these proteins that originate from the intestine had an apparent increase in levels in pancreatic cancer, however, this may have been partly due to decreased levels in pancreatic enzymes and other proteins. Preferred control proteins included any with relatively constant levels between patient, and patient types, and included calcium-activated chloride channel regulator 1 precursor; intestinal-type alkaline phosphatase precursor; sucrase-isomaltase intestinal; and maltase-glucoamylase intestinal.

TABLE 4
Proteins not Exhibiting Significant Fluctuation in Gastrointestinal
Lavage Fluid of Subjects with Pancreatic Cancer

| | NCBI | | | | MgC** Pancreatic | |
|---|---|---|---|---|---|---|
| SEQ ID | Accession | | MgC** Control | | Cancer | |
| NO.: | Nos. | Protein name | Ranking | Score | Ranking | Score |
| 32 | 157266300 | aminopeptidase N precursor | 6 | 3633 | 25 | 1589 |
| 33 | 110611231 | calcium -activated chloride | 9 | 2731 | 6 | 4412 |
| | | channel regulator 1 precursor | | | | |
| 34 | 157266292 | intestinal-type alkaline | 91 | 339 | 54 | 830 |
| | | phosphatase precursor | | | | |
| 35 | 223942069 | enteropeptidase precursor | 43 | 656 | 43 | 914 |
| 36 | 18765694 | dipeptidyl peptidase 4 | 33 | 970 | 36 | 1125 |
| 37 | 153070264 | meprin A subunit beta | 27 | 1164 | 47 | 885 |
| | | precursor | | | | |
| 38 | 153070262 | meprin A subunit alpha | 31 | 1030 | 23 | 1713 |
| | | precursor | | | | |
| 39 | 157364974 | sucrase-isomaltase intestinal | 23 | 1290 | 15 | 1930 |
| 40 | 221316699 | maltase-glucoamylase | 12 | 2434 | 7 | 3811 |
| | | intestinal | | | | |

**MgC = magnesium citrate

Other proteins whose levels were found to either decrease or increase in cancer are shown in Table 5. Alpha-1-antitrypsin may originate from blood while other proteins listed were not typically detected in serum/plasma samples.

TABLE 5

| | NCBI | | | | MgC** Pancreatic | |
|---|---|---|---|---|---|---|
| SEQ ID | Accession | | MgC** Control | | Cancer | |
| NO.: | Nos. | Protein name | Ranking | Score | Ranking | Score |
| 41 | 7669492 | glyceraldehyde-3-phosphate | 7 | 2793 | 30 | 1280 |
| | | dehydrogenase | | | | |
| 42 | 10334859 | creatine kinase U-type | 44 | 656 | — | |
| | | mitochondrial precursor | | | | |
| 23 | 50363217 | alpha-1-antitrypsin precursor | 16 | 1940 | 9 | 3440 |
| 44 | 110618248 | cadherin-related family | 54 | 501 | — | |
| | | member 5 isoform 1 precursor | | | | |
| 45 | 148539840 | deleted in malignant brain | 57 | 471 | — | |
| | | tumors 1 protein isoform a | | | | |
| | | precursor | | | | |
| 46 | 285002214 | cadherin-related family | 41 | 785 | 70 | 726 |
| | | member 2 precursor | | | | |
| 47 | 98986445 | carcinoembryonic antigen- | 222 | 221 | 34 | 1152 |
| | | related cell adhesion molecule | | | | |
| | | 5 preproprotein | | | | |
| 48 | 4502517 | carbonic anhydrase 1 | 487 | 162 | 31 | 1273 |

**MgC = magnesium citrate

### Example 2

### Analysis of Biomarkers in Patients

MS analysis indicating target protein position for gastrointestinal lavage fluid samples from four patients with pancreatic cancer was compared to four normal volunteers. For ELISA analysis, gastrointestinal lavage fluid collected from patients and volunteers was diluted ten-fold with phosphate buffered saline (lx PBS) and analyzed with commercial ELISA methods for some of the proteins and markers detected by MS as well as for known cancer associated antigens. These included pancreatic amylase (ARUP Test #20506, ARUP Laboratories, Salt Lake City, Utah), pancreatic lipase (ARUP Test #20715, ARUP Laboratories, Salt Lake City, Utah), carcinoembryonic antigen (CEA) (ARUP Test #20746, ARUP Laboratories, Salt Lake City, Utah), CA19-9 (ARUP Test #20746, ARUP Laboratories, Salt Lake City, Utah) and trypsin-like immunoreactivity (ARUP Test #70003, ARUP Laboratories, Salt Lake City, Utah). ELISA analyses showed agreement with mass spectrometry where the amounts of pancreatic enzymes in general were reduced and other proteins increased. The results for MS data and ELISA data are summarized in Tables 6 and 7, respectively.

TABLE 6
Mass Spectrometry Analysis

| | NCBI | | MS Posn for | | | | MS Posn for | | | |
|---|---|---|---|---|---|---|---|---|---|---|
| SEQ ID | Accession | | cancer patients | | | | control patients | | | |
| NO.: | Nos. | Protein name | PC1 | PC2 | PC3 | PC4 | C1 | C2 | C3 | C1 |
| 1 | 10835000 | pancreatic | ND | ND | ND | ND | 2 | 1 | 4 | 1 |
| | | triacylglycerol | | | | | | | | |
| | | lipase precursor | | | | | | | | |
| 2 | 4502085 | pancreatic alpha- | ND | ND | 63 | ND | 7 | 5 | 1 | 2 |
| | | amylase precursor | | | | | | | | |
| 3 | 10280622 | alpha-amylase 2B | ND | ND | ND | ND | 14 | 8 | 2 | 3 |
| | | precursor | | | | | | | | |
| 13 | 4506145 | trypsin-1 | 29 | 18 | 12 | ND | 9 | 33 | 9 | 18 |
| | | preproprotein | | | | | | | | |
| 12 | 4506147 | trypsin-2 | 48 | 26 | 17 | ND | 6 | 28 | 15 | 28 |
| | | preproprotein | | | | | | | | |

TABLE 7
ELISA Analysis

| | | | Concentration | | | | Concentration | | | |
|---|---|---|---|---|---|---|---|---|---|---|
| | NCBI | | in cancer | | | | in control | | | |
| SEQ ID | Accession | Protein name, | patient samples | | | | patient samples | | | |
| NO. | Nos. | concentration units | PC1 | PC2 | PC3 | PC4 | C1 | C2 | C3 | C1 |
| 1 | 10835000 | pancreatic lipase, | <4 | <4 | <4 | <4 | 2793 | 1060 | 4040 | 2525 |
| | | U/L | | | | | | | | |
| 2 | 4502085 | pancreatic amylase, | 32 | 31 | 3 | <3 | 786 | 392 | 1240 | 929 |
| 3 | 10280622 | U/L | | | | | | | | |
| 12 | 4506145 | trypsin-like | 113 | 193 | 74 | 3 | 110 | 178 | 380 | 1586 |
| 13 | 4506147 | immunoreactivity, | | | | | | | | |
| | | ng/mL | | | | | | | | |
| 47 | 98986445 | carcinoembryonic | 807 | 526 | 157 | 1311 | 84 | 80 | 28 | 34 |
| 497 | 40255013 | antigen, ng/mL | | | | | | | | |
| 729 | 121114300 | | | | | | | | | |
| 726 | 296317312 | | | | | | | | | |
| N/A | None | CA19-9, U/mL | 43 | 83 | 43 | 34 | 28 | 23 | 8 | 6 |
| | (Glycan, | | | | | | | | | |
| | not | | | | | | | | | |
| | protein) | | | | | | | | | |

### Example 3

### Collection of Samples

GLF samples were collected from normal volunteers and analyzed by MS. Samples taken early in the bowel cleansing process (following initial induction of copious diarrhea) were compared to samples taken the end of the bowel preparation. The analysis showed that early sample collection yielded results (with respect to protein MS position) similar to the samples collected at the end of the bowel preparation. Thus a full bowel preparation, while desirable to remove stool material, may not be required in particular methods.

### Example 4

### General Materials and Methods: Sample Collection, Preparation, Processing and Analysis

### Control Samples

Control samples were obtained from the Gastrointestinal Laboratory University of South Alabama Medical Center by aspiration of residual gastrointestinal lavage fluid (gastrointenstinal lavage fluid) from the bowels of patients at the beginning of the colonoscopy procedure. Control samples were from routine colonoscopies that were found to be free from adenomas or colorectal cancer and were prepared for colonoscopy using SuPrep (Braintree Laboratories, Braintree, Mass.) per manufacturer's instructions or Polyethylene glycol electrolyte solution (PEG-ELS). Approximately 30 ml of gastrointestinal lavage fluid was aspirated into a mucus trap placed in-line with the endoscope. Immediately after collection, gastrointestinal lavage fluid was transferred to a labeled conical centrifuge tube containing a protease-inhibitor tablet (Complete tablet; Roche, Mannheim, Germany) and stored at −20° C. for no more than 48 hours prior to processing.

### Sample Preparation

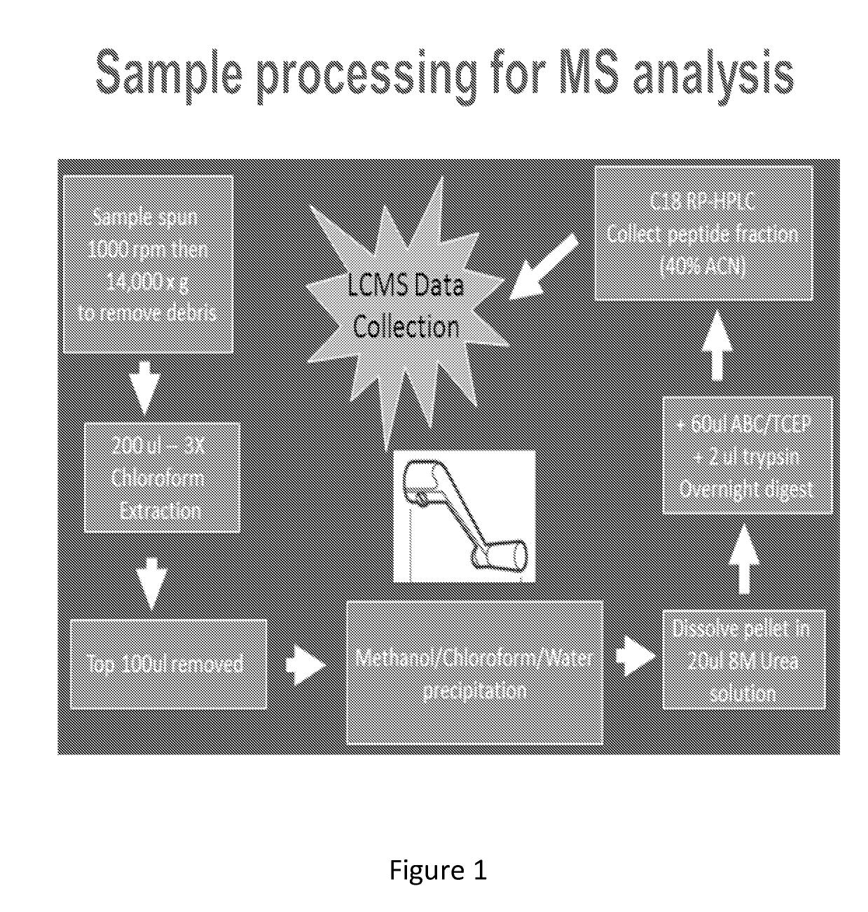

After collection, samples were thawed and immediately centrifuged at 1000 rpm (˜200×g) for 25 minutes to remove large particulates and cells (FIG. 1). The supernatant was then centrifuged again at 14,000×g for 25 minutes to remove small particulates and bacteria. All centrifugation steps were performed in an Eppendorf model 5804 R centrifuge at 4° C.

### Protein Isolation

As further set forth in Example 1, three hundred microliters of each sample was extracted three times with 1 ml of chloroform to remove lipid material and polyethylene glycol. After the final extraction, the aqueous layer was centrifuged at maximum speed for five minutes and 100 μl of the aqueous layer was taken from the top and transferred to a new Eppendorf tube. To precipitate the proteins from the sample, 400 μl of methanol was added to the 100 μl of sample. The sample was centrifuged briefly in a tabletop centrifuge to collect the pellet and 200 μl of chloroform was added to solubilize phospholipids in the methanol layer followed by 300 μl of water to dissolve excess salts and water-soluble pigments. The mixture was vortexed and then centrifuged for five minutes at 13,000×g. This causes the protein to partition at the interface between the aqueous layer, which contains the salts and pigments; and the organic layer, which contains the lipids.

The aqueous layer (about 750 μl) was carefully removed without disturbing the interface and discarded. After this, the protein at the interface was forced to pellet with the addition of 300 μl of methanol. The mixture was vortexed briefly and centrifuged at 13,000×g for five minutes. The supernatant was discarded and the pellet was dried in a speed vac (Savant, Thermo) for ten minutes. The protein pellet was resuspended in a 20 μl of 8 M urea, 10 mM TCEP, 5 mM EDTA, and 0.1 M ABC solution. Once the pellet was completely dissolved, the mixture was diluted with 60 μl of 50 mM ABC/10 mM TCEP and digested with 2 μl of 10 mM sequencing grade trypsin (Promega) overnight on a shaker at 600 rpm at 37° C.

The digest was diluted into an LC vial by adding 75 μl of the digest to 20 μl of water and 75 μl of this mixture was injected onto the C₁₈ pre-column (5 μm; 5 by 0.3 mm; Zorbax; Agilent Technologies) connected to an Agilent 1200 series nano-liquid chromatography (nano-LC) pump and thermostated auto-injector (Agilent Technologies, Santa Clara, Calif.). Solvent A was 2% acetonitrile and 0.05% TFA in water, and solvent B consisted of 2% water and 0.05% TFA in acetonitrile. A flow rate of 200 μl/minute was maintained throughout the run. For the first 13 minutes, 2% solvent B was used to load the sample onto a C₁₈ pre-column and wash it. From time 15 to 21 minutes the peptides were eluted from the column with 40% B and this fraction is collected. This was followed by a column wash with 90% B from time 22 to 30 minutes and re-equilibration to 2% B in the final minute. The entire run time was 31 minutes. The A280 peak area of the eluted peptide peak was used as an estimate of how much protein was retrieved.

The eluted peptides were dried in a speed vac (Savant, Thermo) and re-dissolved in an amount of 0.1% TFA equal to 1/100 of the area of the A280 peak in μl, with a minimum volume of 50 μl and a maximum volume of 500 μl in order to normalize protein concentrations in the injected samples.

### Mass Spectrometry

Samples were injected in triplicate into an Agilent 1200 series nano-liquid HPLC coupled to a linear ion trap/Orbitrap hybrid MS (LTQ-Orbitrap (Thermo)). The HPLC mobile phases consisted of 3% acetonitrile and 0.2% formic acid in water (solvent A), and 3% water and 0.2% formic acid in acetonitrile (solvent B). A flow rate of 4 μl/minute of 5% solvent B was used to load the sample onto a C₁₈ pre-column (5 μm; 5 by 0.3 mm; Zorbax; Agilent Technologies), and a flow rate of 1 μl/minute was used to elute the sample from the pre-column onto a separating Hypersil Gold C₁₈ chromatography column (30 mm by 0.18 mm; Thermo Fisher Scientific). The linear solvent gradient was slowly ramped to 40% B over 70 min in order to elute the peptides from the column and then to 90% B over the final 20 min to wash the column. The total run time (pre-column and resolving chromatography) for each sample injection was 2 hours. During the 70 minute peptide elution at 40% B, eluted peptides were injected into the nanoflow source of the LTQ for MS-analysis. The LTQ-Orbitrap acquired one MS-only scan (Orbitrap) at a resolution of 60,000, while acquiring up to 5 MS-MS scans (LTQ), with a consistent cycle time of approximately 1 s, using the Xcalibur software program (Thermo Fisher Scientific). Peptide masses selected for fragmentation were then added to an exclusion list (within 10 ppm) to prevent repeated sequencing of abundant peptides for five minutes.

### Example 5

### Data Analysis and Results

MS/MS peptide sequence data obtained from the LTQ-Orbitrap from a representative control gastrointestinal lavage fluid sample collected during colonoscopy and prepared using the standard method described in FIG. 1 above were converted to mascot generic format files (.mgf) and ID matches identified using the Mascot search engine (http://www.matrixscience.com). Protein identifications (with a threshold of 95% confidence) were determined by the Mascot software program. All files were searched against a custom database generated by combining the NCBI RefSeq database with SwissProt Ig sequences (Feb. 8, 2012—33712 sequences; 18670280 residues) using taxonomy: human, enzyme specificity: semi-trypsin, and a mass accuracy of 10 ppm for precursor ions and 0.6 DA for MS/MS data.

Table 8 shows the top 19 hits in order of Mascot Score, which is determined by how closely the data matches the theoretical data generated for that peptide sequence. The higher the score the more accurate the match as well as the more abundant the protein is in the sample.

TABLE 8

| SEQ | | NCBI | | |
|---|---|---|---|---|
| ID | | Accession | | |
| NO: | Rank | No. | Mascot Score | Protein Name |
| 2 | 1 | 4502085 | 1212 | Pancreatic alpha amylase precursor (homo |
| | | | | sapiens) |
| 3 | 2 | 10280622 | 1093 | Alpha-amylase 2B precursor (homo |
| | | | | sapiens) |
| 49 | 3 | 148536848 | 1057 | Bile salt activated lipase precursor (homo |
| | | | | sapiens) |
| 1 | 4 | 10835000 | 1053 | Pancreatic triacylglycerol lipase precursor |
| | | | | (homo sapiens) |
| 6 | 5 | 54607080 | 501 | Carboxypeptidase B preprotein (homo |
| | | | | sapiens) |
| 8 | 6 | 236460050 | 395 | Chymotrypsin-like elastase family |
| | | | | member 3A preprotein (homo sapiens) |
| 50 | 7 | 193806374 | 364 | RecName: Full = Ig mu chain C region |
| 51 | 8 | 113584 | 354 | RecName: Full = Ig alpha-1 chain C region |
| 13 | 9 | 4506145 | 335 | Trypsin-1 preprotein (homo sapiens) |
| 52 | 10 | 31377806 | 323 | Polymeric immunoglobulin receptor |
| | | | | precursor (homo sapiens) |
| 4 | 11 | 4502997 | 297 | Carboxypeptidase A1 precursor |
| 10 | 12 | 15559207 | 280 | Chymotrypsin-like elastase family |
| | | | | member 2A preprotein (homo sapiens) |
| 53 | 13 | 218512088 | 273 | RecName: Full = Ig alpha-2 chain C region |
| 54 | 14 | 341913702 | 269 | PREDICTED: deleted in malignant brain |
| | | | | tumors 1 protein isoform 2 |
| 12 | 15 | 4506147 | 218 | Trypsin-2 preprotein (homo sapiens) |
| 11 | 16 | 6679625 | 206 | Chymotrypsin-like elastase family |
| | | | | member 3B preprotein (homo sapiens) |
| 55 | 17 | 118498341 | 196 | Chymotrypsinogen B precursor (homo |
| | | | | sapiens) |
| 23 | 18 | 50363217 | 166 | Alpha-1-antitrypsin precursor (homo |
| | | | | sapiens) |
| 7 | 19 | 217416390 | 165 | Carboxypeptidase A2 precursor (homo |
| | | | | sapiens) |

### Mass, Time, and Intensity Data

The intensity of detected peptides was calculated based on MS data using an approach similar to the Accurate Mass Tag (AMT) method developed by Smith and coworkers (Conrads, T. P. et al., (2000) Anal. Chem. 72, 3349-3354; Strittmatter, E. F. et al., (2003) J. Am. Soc. Mass Spectrom. 14, 980-991). A program called DifProWare, a Web-based platform developed at the University of South Alabama (available at http://mciproteomics.usouthal.edu/difproware/) (Tucker, A. M. et al., (2011) Appl. Environ. Microbiol. 77, 4712-4718), was used to generate the mass, time, and intensity data for analysis. Briefly, MS/MS peptide sequence data were converted to mascot generic format files (.mgf) and matches identified using the Mascot search engine (http://www.matrixscience.com). Protein identifications (with a threshold of 95% confidence) were determined by the Mascot software program. All files were searched against a custom database generated by combining the NCBI RefSeq database with SwissProt Ig sequences (Feb. 8, 2012—33712 sequences; 18670280 residues) using taxonomy: human, enzyme specificity: semi-trypsin, and a mass accuracy of 10 ppm for precursor ions and 0.6 DA for MS/MS data.

The MS-only data were examined using the ReSpect algorithm (Positive Probability, Ltd., Isleham, United Kingdom). This algorithm deconvolves detected peaks, converts electrospray mass spectra to zero-charge spectra, and corrects baselines, improving signal-to-noise ratios. The raw MS-only isotopic data are processed, generating a file containing deconvoluted mass, time, intensity, and probability statistics. Peptides were only accepted for analyses if they had an isotopic profile agreement confidence level of >95%. The Mascot ID information for each peptide as well as its mass, time, and intensity data in each sample being compared is combined within DifProWare and the resulting file is a comma-separated spreadsheet file associating peptide mass, time, intensity, and ID data.

### Peptide to Protein Rollup

Protein abundances were calculated from the individual peptide abundances using the Rollup algorithm implemented in DanteR 0.2 (Taverner, T. et al., (2012) Bioinformatics 28, 2404-2406; Polpitiya, A. D. et al., (2008) Bioinformatics 24, 1556-1558) running under R 32-bit version 2.15.2 (R Development Core Team. (2013) R: a language and environment for statistical computing. R Foundation for Statistical Computing, Vienna, Austria. http://www.R-project.org) under Windows 7. DanteR is an open source software package that was developed by Tom Taverner and Ashoka Polpitiya at the Pacific Northwest National Laboratory to analyze proteomic data generated using the accurate mass and time tag approach.

This process combines intensity information from individual peptides into a single “intensity” for their identified protein. A brief summary of the process is as follows: For each group of peptides belonging to a single protein, the peptide with the highest overall abundance across all samples is chosen as a reference peptide. All peptides belonging to that protein are then expressed as a ratio to the reference value. The median ratio for each peptide across all samples is also calculated and the median ratio is subtracted from each peptide ratio. Outliers are then detected using Grubb's test and removed, and the median value of remaining selected peptide intensities is used to calculate the protein intensity.

The rollup was performed with the following parameters: rolling up using NCBI Accession number, minimum presence of at least one peptide at 50%, mode median, minimum dataset presence of three peptides, minimum number of peptides required for Grubb's test of 5, and p-value cutoff for Grubb's test at 0.05. The resulting spreadsheet of identified proteins and relative abundances was used in the subsequent statistical analyses.

### Comparison of Home Collected Versus Clinic Collected Samples

In order to prove that major proteins were unaffected by the difference in the collection method between the home collected samples and the clinic collected samples, an experiment was performed comparing 44 matched pairs of samples. In one set, samples are self-collected by the subject via the toilet collection container method in which a hat is placed on a toilet seat for collection of gastrointestinal lavage fluid and transferred to tube with inhibitor immediately prior to colonoscopy (“hat samples”). In another set, samples are collected during colonoscopy through an endoscope (“scope samples”). Protein intensity values of the 44 “hat” and “scope” samples were obtained from LC-MS/MS data using the peptide to peptide rollup procedure described above and were compared using the Mann Whitney U test. The p-values are shown in Table 9 for three of the major proteins in gastrointestinal lavage fluid: carboxypeptidase B, pancreatic tracylglycerol lipase and chymotrypsin-like elastase family member 2A, demonstrating the differences were not significant.

TABLE 9
Comparison of Hat vs. Scope Collections

| | NCBI | | | |
|---|---|---|---|---|
| SEQ | Accession | # | | |
| ID NO. | Nos. | Peptides | Protein ID | P value |
| 6 | 54607080 | 30 | Carboxypeptidase B | 0.90368 |
| 1 | 10835000 | 41 | Pancreatic triacylglycerol lipase | 0.40868 |
| 10 | 15559207 | 15 | Chymotrypsin-like elastase | 0.84777 |
| | | | family member 2A | |

Overall comparisons of changes in individual hat and scope pairs were no greater than changes seen in replicates of the same samples. Therefore, the collection method does not affect the data and the two methods may be used interchangeably and compared.

### Reproducibility of Methodology

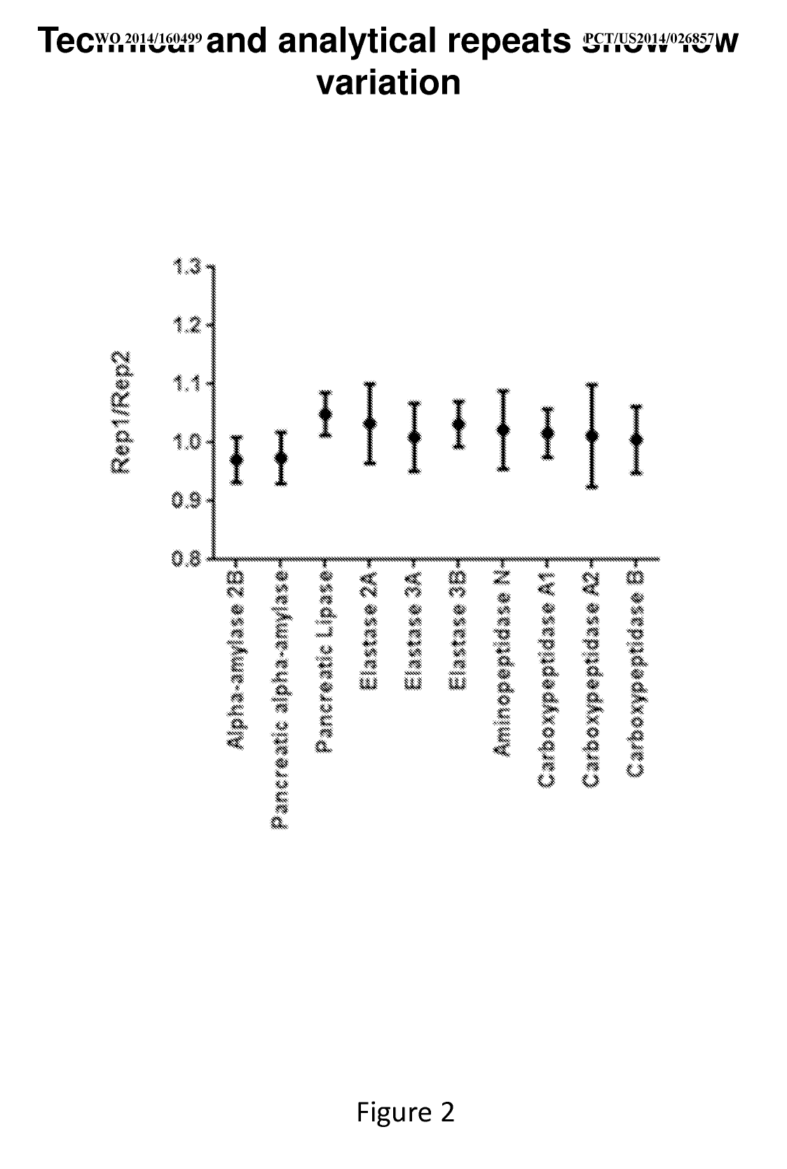

One control sample was processed six times according to the standard methods described previously and the ratios of the intensities of the indicated proteins were analyzed between all pairs of replicates (36 combinations) (FIG. 2). A ratio of 1=identical. Bars show the 5-95% range of the ratios. Analytical replicates did not vary from each other by more than ˜20%. The data demonstrated that the profiles of key proteins as shown in FIG. 2 showed little variation, and that the method is highly reproducible. 2-sigma confidence level is shown.

Comparison of Abundance of Proteins in GLF of Subjects with Resectable Pancreatic Ductal Adenocarcinoma Versus Abundance of Proteins in GLF of Healthy Subjects

PDAC gastrointestinal lavage fluid samples were collected from 27 cases of resectable PDAC patients in pre-op prior to surgery. Patients had been bowel prepped with two bottles of magnesium citrate solution the previous night and had not eaten or drank since midnight the night before the sample was taken. Patients were asked to defecate into a collection container that fits over the toilet, and the gastrointestinal lavage fluid was transferred to a labeled conical centrifuge tube containing a protease inhibitor tablet (Complete tablet; Roche, Mannheim, Germany) and transported to the laboratory immediately on ice.

The average rankings of the top pancreatic proteins in gastrointestinal lavage fluid were compared between these 27 PDAC patients and 121 control gastrointestinal lavage fluid samples collected at colonoscopy as described previously using the ranking of protein abundance as determined by Mascot as described previously above. The ranking of the pancreatic proteins was significantly decreased in the PDAC group as compared to the control group (p<1.0E-09) (Table 10). Average intensities calculated using the rollup algorithm as described above were also compared and the fold change indicated.

TABLE 10
Relative abundance of proteins detected in the GLF between healthy
control N = 121 and resectable PDAC (N = 27) cases out of >300 detectable proteins
(ND = non-detected)

| | | | | | Intensity |
|---|---|---|---|---|---|
| | | | | | Fold |
| | NCBI | | | | Decrease |
| SEQ ID | Accession | | Healthy | PDAC | PDAC/ |
| NO. | Nos. | Protein ID | Rank | Rank | Healthy |
| 1 | 10835000 | Pancreatic triacylglycerol lipase | 1 | 391 | 226 |
| 2 | 4502085 | Pancreatic alpha-amylase | 2 | 21 | 7 |
| 3 | 10280622 | Alpha-amylase 2B | 3 | 22 | 18 |
| 4 | 4502997 | Carboxypeptidase A1 | 4 | 160 | 10 |
| 56 | 56549662 | Alpha-amylase 1 | 5 | 20 | 10 |
| 6 | 54607080 | Carboxypeptidase B | 10 | 163 | 25 |
| 7 | 217416390 | Carboxypeptidase A2 | 11 | ND | 27 |
| 8 | 236460050 | Chymotrypsin-like elastase | 17 | 124 | 38 |
| | | family member 3A | | | |
| 9 | 62526043 | Chymostrypsin-C | 19 | ND | 53 |
| 10 | 15559207 | Chymotrypsin-like elastase | 21 | 126 | 6 |
| | | family member 2A | | | |
| 11 | 6679625 | Chymotrypsin-like elastase | 24 | 107 | 7 |
| | | family member 3B | | | |

*12 of the PDAC cases showed no evidence of ductal dilation

### Analysis of Amylase and Lipase Via Spectrometry and ELISA

GLF samples obtained from three control samples obtained by colonoscopy and three of the PDAC samples obtained prior to surgery were diluted 10× in PBS and analyzed for amylase and lipase using standard ELISA methods which measure units of enzyme per liter. The data demonstrated a greater than 250 fold decrease in lipase and a greater than 3.7 fold decrease in amylase between the PDAC and control samples. Furthermore the MS data and the ELISA data were concordant. MS values are denoted with Mascot scores, determined as described previously above.

TABLE 11
Comparison of Amylase and Lipase Assessed via Mass Spectrometry (MS)
vs. ELISA in Healthy and PDAC samples

| | | | Amylase | Lipase 78974 |
|---|---|---|---|---|
| | Test Subject # | Assay | 78914 (μ/L) | (μ/L) |
| Healthy | 1 | ELISA | 1240 | 4040 |
| | | MS | 1212 | 1053 |
| | 2 | ELISA | 929 | 2525 |
| | | MS | 1774 | 2297 |
| | 3 | ELISA | 4114 | 2020 |
| | | MS | 2333 | 2386 |
| PDAC | 4 | ELISA | 32 | <4 |
| | | MS | 0 | 0 |
| | 5 | ELISA | 199 | <4 |
| | | MS | 447 | 0 |
| | 6 | ELISA | 250 | <4 |
| | | MS | 251 | 0 |
| | 7 | ELISA | <3 | <4 |
| | | MS | 0 | 0 |

### Comparison of Pancreatic Proteins in Pancreatic Juice as Compared to in Gastrointestinal Lavage Fluid

The average rankings of the top pancreatic proteins in pancreatic juice collected directly from the pancreatic duct during surgery in six PDAC patients (labeled “pc”), and one patient determined to have an intraductal papillary mucinous neoplasm (labeled “IPMN 75”) (which is a benign lesion than may progress to PDAC if left untreated) were compared to pancreatic juice from three patients found to have benign pancreatic cysts at surgery (labeled “cyst”). Samples were compared as described previously using the ranking of protein abundance as determined by Mascot as described previously above. The ranking of the pancreatic proteins was significantly decreased in the pancreatic juice from the PDAC group as demonstrated previously in gastrointestinal lavage fluid. This shows that pancreatic proteins are reduced in both the direct pancreatic secretions as well as the gastrointestinal lavage fluid. The proteins were still present in the benign IPMN and in the benign cyst cases. Results are depicted in Table 12.

TABLE 12
Comparison of Pancreatic Proteins in Pancreatic Juice as Compared to in Gastrointestinal Lavage Fluid

| | NCBI | | cyst | cyst | cyst | ipmn | pc | pc | pc | pc | pc | pc |
|---|---|---|---|---|---|---|---|---|---|---|---|---|
| SEQ ID | Accession | | 55 | 61 | 69 | 75 | 29 | 5 | 30 | 44 | 47 | 70 |
| NO. | Nos. | Protein name | Posn | Posn | Posn | Posn | Posn | Posn | Posn | Posn | Posn | Posn |
| 49 | 148536848 | bile salt-activated | 4 | 3 | 5 | 3 | 60 | 30 | ND | 162 | ND | 9 |
| | | lipase precursor | | | | | | | | | | |
| 1 | 10835000 | pancreatic | 3 | 4 | 7 | 7 | ND | 81 | ND | 58 | ND | 24 |
| | | triacylglycerol | | | | | | | | | | |
| | | lipase precursor | | | | | | | | | | |
| 12 | 4506147 | trypsin-2 | 13 | 5 | 14 | 6 | 23 | 12 | 34 | 24 | 11 | 21 |
| | | preproprotein | | | | | | | | | | |
| 13 | 4506145 | trypsin-1 | 6 | 8 | 8 | 5 | 21 | 13 | 25 | 22 | 4 | 17 |
| | | preproprotein | | | | | | | | | | |
| 55 | 118498341 | chymotrypsinogen | 8 | 9 | 15 | 8 | 36 | 14 | ND | 16 | 26 | 20 |
| | | B precursor | | | | | | | | | | |
| 57 | 106507261 | pancreatic lipase- | 16 | 11 | 49 | 13 | ND | ND | ND | 85 | ND | 91 |
| | | related protein 2 | | | | | | | | | | |
| | | precursor | | | | | | | | | | |
| 793 | 342672030 | trypsin-3 isoform 3 | 18 | 12 | 22 | 14 | ND | ND | ND | ND | ND | ND |
| | | preproprotein | | | | | | | | | | |
| 8 | 236460050 | chymotrypsin-like | 11 | 14 | 18 | 9 | 225 | ND | ND | 26 | 28 | 23 |
| | | elastase family | | | | | | | | | | |
| | | member 3A | | | | | | | | | | |
| | | preproprotein | | | | | | | | | | |
| 2 | 4502085 | pancreatic alpha- | 10 | 15 | 12 | 12 | 46 | ND | ND | 31 | 3 | 28 |
| | | amylase precursor | | | | | | | | | | |
| 10 | 15559207 | chymotrypsin-like | 20 | 16 | 23 | 30 | ND | ND | ND | ND | ND | ND |
| | | elastase family | | | | | | | | | | |
| | | member 2A | | | | | | | | | | |
| | | preproprotein | | | | | | | | | | |
| 3 | 10280622 | alpha-amylase 2B | 14 | 19 | 13 | 15 | ND | ND | ND | ND | 7 | ND |
| | | precursor | | | | | | | | | | |
| 4 | 4502997 | carboxypeptidase | 21 | 20 | 31 | 21 | ND | 99 | ND | 78 | 42 | ND |
| | | A1 precursor | | | | | | | | | | |
| 6 | 54607080 | carboxypeptidase | 17 | 22 | 16 | 22 | 37 | 15 | ND | 19 | 22 | 31 |
| | | B preproprotein | | | | | | | | | | |
| 23 | 50363217 | alpha-1-antitrypsin | 23 | 23 | 10 | 33 | 12 | 20 | 26 | 12 | 2 | 18 |
| | | precursor | | | | | | | | | | |
| 11 | 6679625 | chymotrypsin-like | 22 | 24 | 30 | 16 | ND | ND | ND | ND | 27 | ND |
| | | elastase family | | | | | | | | | | |
| | | member 3B | | | | | | | | | | |
| | | preproprotein | | | | | | | | | | |
| 9 | 62526043 | chymotrypsin-C | 27 | 28 | 55 | 36 | ND | ND | ND | ND | ND | ND |
| | | preproprotein | | | | | | | | | | |
| 7 | 217416390 | carboxypeptidase | 31 | 29 | ND | 31 | ND | ND | ND | ND | ND | ND |
| | | A2 precursor | | | | | | | | | | |
| 58 | 58331211 | chymotrypsin-like | 29 | 35 | ND | 53 | ND | ND | ND | ND | ND | ND |
| | | elastase family | | | | | | | | | | |
| | | member 2B | | | | | | | | | | |
| | | preproprotein | | | | | | | | | | |

Assessment of Gastrointestinal Lavage Fluid Samples from Subjects with Pancreatic Ductal Adenocarcinoma in Head of Pancreas

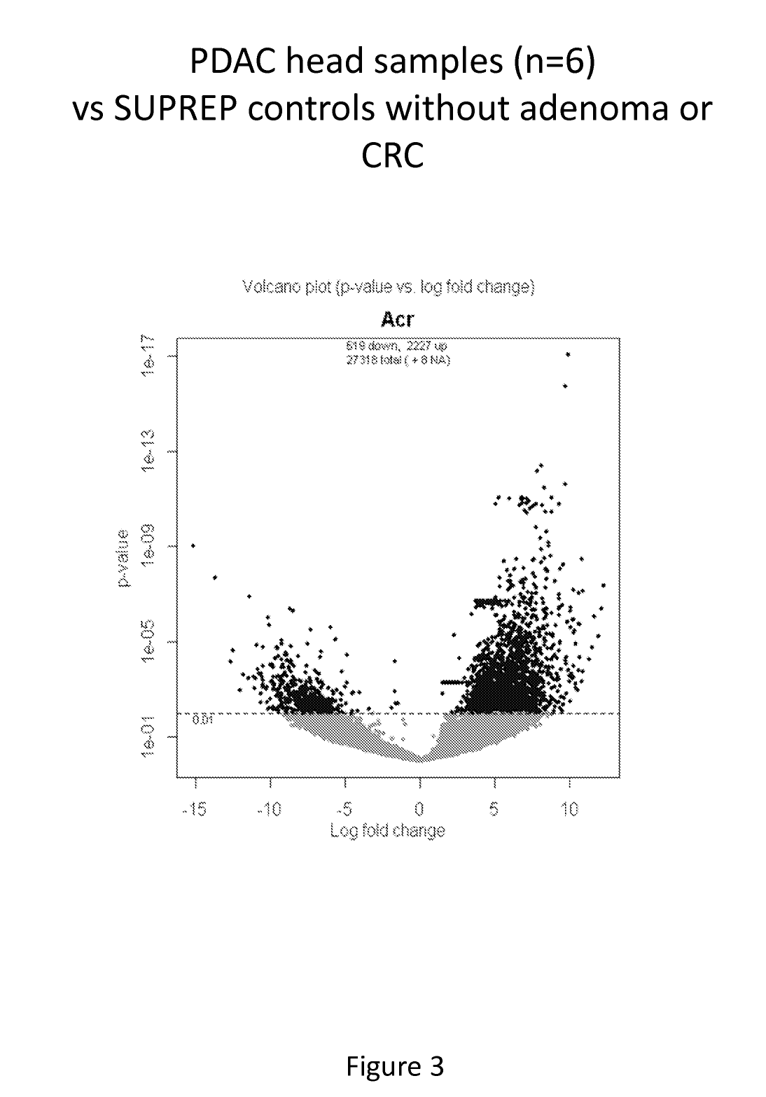

A second group of PDAC patient gastrointestinal lavage fluid samples were obtained. Patients with pancreatic masses detected using imaging were recruited to the study. gastrointestinal lavage fluid samples were collected after detection of the mass but prior to surgery. Those who were subsequently found to have pancreatic ductal adenocarcinoma in the head of the pancreas (n=6) were selected for comparison to the controls. The patient was provided with a kit to take home that included a dose of SuPrep bowel preparation solution (Braintree Laboratories, Braintree, Mass.), a collection container that fits over the toilet, a labeled conical centrifuge tube containing a protease inhibitor tablet (Complete tablet; Roche, Mannheim, Germany), and a disposable pipette for transfer of sample from toilet collection container to conical tube. The patient collected a sample of clear gastrointestinal lavage fluid and shipped it frozen on ice to the laboratory for analysis. The sample was prepared in the same manner as the previously obtained controls that were collected at colonoscopy as described previously. Data were processed using the standard approach described previously, with the exception of a 2 group ANOVA (t-test) in DanteR being used for comparison instead of the Mann Whitney U test. The intensity values of the individual peptides prior to “rollup” into protein values between 81 control samples that had been bowel-prepared with SuPrep and the 6 PDAC head samples that were also bowel-prepared using SuPrep were compared (FIG. 3). Of the 27,318 peptides were analyzed, 619 were significantly decreased in the PDAC cases (p<0.01). In contrast, 2227 peptides were significantly increased in the PDAC cases (p<0.01). Many of the peptides were unidentified and may contain post-translational modifications or mutations that may cause mass shifts.

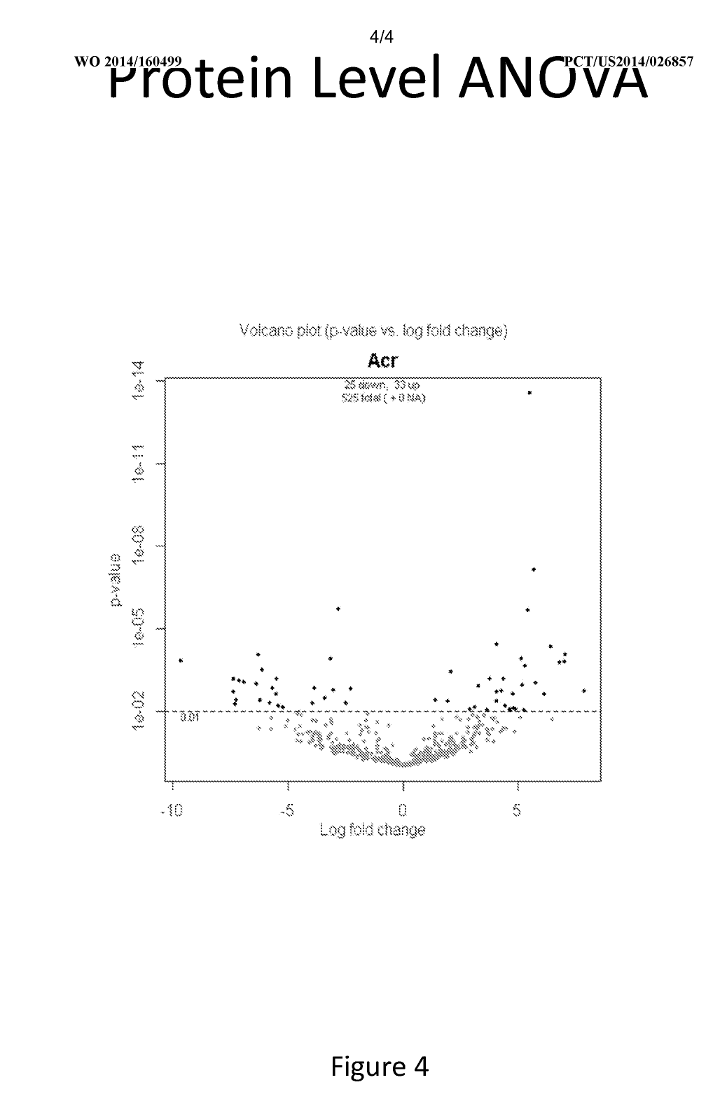

The peptide intensity data was “rolled up” into protein intensity data as described above. Intensities of all proteins were compared between the 81 control samples that had been bowel-prepared using SuPrep and the 6 PDAC head samples that were also bowel-prepared using SuPrep using a 2 group ANOVA (t-test) in DanteR (FIG. 4). The data demonstrated that 25 peptides significantly decreased and 33 peptides significantly increased in the PDAC cases.

Table 13 depicts the rolled up intensity values of proteins present in the 6 PDAC head as compared to the 81 control samples. Log 2 Fold changes and p-values as determined by protein level ANOVA are shown.

TABLE 13
PDAC in the Head of the Pancreas

| | NCBI | | | |
|---|---|---|---|---|
| | Accession | | Log_2 | |
| SEQ ID NO. | Nos. | Protein | change | P value |
| 59 | 189083692 | Fructose-1:6-bisphosphatase 1 | 5.39 | 4.23E−06 |
| 20 | 4502027 | Serum Albumin Preprotein | 3.43 | .0003 |
| 10 | 15559207 | Chymotrypsin-like elastase family | −5.36 | .0008 |
| | | member 2A preprotein | | |
| 60 | 11225609 | Angiotensin-converting enzyme 2 | 3.75 | .0024 |
| | | precursor | | |
| 16 | 6679625 | Chymotrypsin-like elastase family | −1.82 | .0025 |
| | | member 3B preprotein | | |
| 62 | 93141226 | Xaa-Pro aminopeptidase 2 precursor | 3.99 | .0065 |
| 58 | 58331211 | Chymotrypsin-like elastase family | −6.15 | .0189 |
| | | member 2B preprotein | | |
| 6 | 54607080 | Carboxypeptidase B preprotein | −2.18 | .0189 |
| 40 | 221316699 | Maltase-glucoamylase: intestinal | 1.81 | .0398 |

A similar experiment was performed with respect to gastrointestinal lavage fluid obtained from 3 subjects with neuroendocrine tumors present in the tail of the pancreas and compared to the 81 control samples, per the methods and analysis described above. Table 14 depicts the rolled up intensity values of proteins present in the gastrointestinal lavage fluid obtained from these subjects as compared to the 81 control samples. Log 2 Fold changes and p-values as determined by protein level ANOVA are shown.

TABLE 14
Neuroendocrine Cancer in the Tail of the Pancreas

| | NCBI | | | |
|---|---|---|---|---|
| | Accession | | Log_2 | |
| SEQ ID NO. | Nos. | Protein | change | P value |
| 62 | 93141226 | Xaa-Pro aminopeptidase 2 precursor | 8.42 | 2.26E−05 |
| 63 | 308736985 | Mucin 13 precursor [homo sapiens] | 11.66 | .0002 |
| 64 | 4503273 | Angiotensin-converting enzyme | 2.79 | .0002 |
| | | isoform 1 precursor | | |
| 40 | 221316699 | Maltase-glucoamylase: intestinal | 3.89 | .0016 |
| 56 | 56549662 | Alpha-amylase 1 precursor | 4.65 | .0024 |
| 3 | 10280622 | Alpha amylase 2B precursor | 4.51 | .0051 |
| 2 | 4502085 | Pancreatic alpha-amylase precursor | 6.30 | .0059 |
| 9 | 62526043 | Chymotrypsin-C-preprotein | 5.52 | .0060 |
| 41 | 7669492 | Glyceraldehyde-3 phosphate | 4.29 | .0075 |
| | | dehydrogenase | | |
| 66 | 132814467 | Glutamyl aminopeptidase | 6.91 | .0091 |

As set forth in Tables 13 and 14, some pancreatic proteins were significantly decreased in PDAC cases but increased or unchanged in neuroendocrine cases.

Table 15 depicts the mascot positions (ranks) of major pancreatic enzymes, intestinal proteins, and serum proteins (Albumin and AAT) compared between the average of 6 PDAC head samples and the average of the 3 neuroendocrine tail pancreatic cancer samples, collected and processed as described above.

TABLE 15

| | | | | |
|---|---|---|---|---|
| | NCBI | | | |
| SEQ ID | Accession | | Head | Tail |
| NO. | Nos. | Protein | PDAC | Neuroendocrine |

Pancreatic Enzymes

| | | | | |
|---|---|---|---|---|
| 2 | 4502085 | Pancreatic alpha-amylase | 10 | 1 |
| | | precursor | | |
| 3 | 10280622 | Alpha-amylase 2B precursor | 0 | 2 |
| 5 | 40254482 | Alpha-amylase 1 precursor | 12 | 3 |
| 1 | 10835000 | Pancreatic triacylglycerol lipase | 8 | 4 |
| | | precursor | | |
| 8 | 236460050 | Chymotrypsin-like elastase family | 29 | 9 |
| | | member 3A preprotein | | |
| 11 | 6679625 | Chymotrypsin-like elastase family | 44 | 17 |
| | | member 3B preprotein | | |
| 13 | 4506145 | Trypsin-1 preprotein | 47 | 18 |

Intestinal Proteins

| | | | | |
|---|---|---|---|---|
| 39 | 157364974 | Sucrose-isomaltase intestinal | 11 | 7 |
| 32 | 157266300 | Aminopeptidase N precursor | 51 | 8 |
| 40 | 221316699 | Maltase glucoamylase intestinal | 7 | 12 |
| 33 | 110611231 | Calcium-activated chloride | 14 | 1 |
| | | channel regulator 1 precursor | | |

Albumin and AAT

| | | | | |
|---|---|---|---|---|
| 20 | 4502027 | Serum albumin preprotein | 2 | 36 |
| 23 | 50363217 | Alpha-1-antitrypsin precursor | 3 | 6 |

Table 16 provides a complete list of proteins that change between PDAC and control cases. Table 16 reflects changes in more than just pancreatic enzymes.

TABLE 16
Complete List of Proteins that Change between PDAC and Control Cases

| | NCBI | | | | | |
|---|---|---|---|---|---|---|
| SEQ ID | Accession | | Head | | Tail | |
| NO: | No. | Protein name | Posn | Score | Posn | Score |
| 2 | 4502085 | pancreatic alpha-amylase precursor | 10 | 3554 | 1 | 5890 |
| | | [Homo sapiens] | | | | |
| 3 | 10280622 | alpha-amylase 2B precursor [Homo sapiens] | 0 | 0 | 2 | 5626 |
| 5 | 40254482 | alpha-amylase 1 precursor [Homo sapiens] | 12 | 3320 | 3 | 5557 |
| 1 | 10835000 | pancreatic triacylglycerol lipase | 8 | 4689 | 4 | 3575 |
| | | precursor [Homo sapiens] | | | | |
| 68 | 154146262 | IgGFc-binding protein precursor [Homo sapiens] | 5 | 5354 | 5 | 2765 |
| 23 | 50363217 | alpha-1-antitrypsin precursor [Homo sapiens] | 3 | 5912 | 6 | 2464 |
| 39 | 157364974 | sucrase-isomaltase intestinal [Homo sapiens] | 11 | 3542 | 7 | 2267 |
| 32 | 157266300 | aminopeptidase N precursor [Homo sapiens] | 51 | 824 | 8 | 2088 |
| 8 | 236460050 | chymotrypsin-like elastase family | 29 | 1272 | 9 | 1959 |
| | | member 3A preproprotein [Homo sapiens] | | | | |
| 53 | 218512088 | RecName: Full = Ig alpha-2 chain C region | 4 | 5447 | 10 | 1878 |
| 51 | 113584 | RecName: Full = Ig alpha-1 chain C region | 1 | 7047 | 11 | 1626 |
| 40 | 221316699 | maltase-glucoamylase intestinal [Homo sapiens] | 7 | 5117 | 12 | 1599 |
| 33 | 110611231 | calcium-activated chloride channel | 14 | 2356 | 13 | 1528 |
| | | regulator 1 precursor [Homo sapiens] | | | | |
| 50 | 193806374 | RecName: Full = Ig mu chain C region | 13 | 2856 | 14 | 1499 |
| 69 | 125145 | RecName: Full = Ig kappa chain C region | 9 | 3684 | 15 | 1383 |
| 52 | 31377806 | polymeric immunoglobulin receptor | 6 | 5266 | 16 | 1089 |
| | | precursor [Homo sapiens] | | | | |
| 11 | 6679625 | chymotrypsin-like elastase family | 44 | 904 | 17 | 983 |
| | | member 3B preproprotein [Homo sapiens] | | | | |
| 13 | 4506145 | trypsin-1 preproprotein [Homo sapiens] | 47 | 873 | 18 | 942 |
| 6 | 54607080 | carboxypeptidase B preproprotein | 17 | 1696 | 19 | 871 |
| | | [Homo sapiens] | | | | |
| 10 | 15559207 | chymotrypsin-like elastase family | 60 | 749 | 20 | 826 |
| | | member 2A preproprotein [Homo sapiens] | | | | |
| 9 | 62526043 | chymotrypsin-C preproprotein [Homo sapiens] | 65 | 723 | 21 | 822 |
| 12 | 4506147 | trypsin-2 preproprotein [Homo sapiens] | 48 | 866 | 22 | 821 |
| 55 | 118498341 | chymotrypsinogen B precursor [Homo sapiens] | 20 | 1492 | 23 | 776 |
| 70 | 291045225 | titin isoform N2-A [Homo sapiens] | 39 | 933 | 24 | 736 |
| 71 | 291045230 | titin isoform novex-2 [Homo sapiens] | 38 | 940 | 25 | 705 |
| 64 | 4503273 | angiotensin-converting enzyme isoform | 52 | 820 | 26 | 690 |
| | | 1 precursor [Homo sapiens] | | | | |
| 72 | 119220571 | pancreatic secretory granule membrane | 1256 | 162 | 27 | 671 |
| | | major glycoprotein GP2 isoform 2 | | | | |
| | | precursor [Homo sapiens] | | | | |
| 49 | 148536848 | bile salt-activated lipase precursor | 21 | 1478 | 28 | 624 |
| | | [Homo sapiens] | | | | |
| 36 | 18765694 | dipeptidyl peptidase 4 [Homo sapiens] | 40 | 931 | 29 | 597 |
| 73 | 256222411 | filamin-B isoform 1 [Homo sapiens] | 86 | 561 | 30 | 552 |
| 37 | 153070264 | meprin A subunit beta precursor [Homo sapiens] | 37 | 984 | 31 | 522 |
| 74 | 21489959 | immunoglobulin J chain precursor | 26 | 1435 | 32 | 502 |
| | | [Homo sapiens] | | | | |
| 46 | 285002214 | cadherin-related family member 2 | 16 | 1748 | 33 | 488 |
| | | precursor [Homo sapiens] | | | | |
| 38 | 153070262 | meprin A subunit alpha precursor | 33 | 1147 | 34 | 481 |
| | | [Homo sapiens] | | | | |
| 75 | 4507725 | transthyretin precursor [Homo sapiens] | 15 | 1773 | 35 | 409 |
| 20 | 4502027 | serum albumin preproprotein [Homo sapiens] | 2 | 6899 | 36 | 407 |
| 18 | 83367077 | mucin-16 [Homo sapiens] | 72 | 639 | 37 | 394 |
| 76 | 121039 | RecName: Full = Ig gamma-1 chain C region | 41 | 915 | 38 | 386 |
| 77 | 148833506 | obscurin isoform b [Homo sapiens] | 94 | 546 | 39 | 374 |
| 17 | 16306550 | selenium-binding protein 1 [Homo sapiens] | 31 | 1190 | 40 | 373 |
| 4 | 4502997 | carboxypeptidase A1 precursor [Homo sapiens] | 18 | 1672 | 41 | 373 |
| 78 | 118572606 | hemicentin-1 precursor [Homo sapiens] | 109 | 505 | 42 | 366 |
| 79 | 341913700 | PREDICTED: deleted in malignant brain tumors | 0 | 0 | 43 | 363 |
| | | 1 protein isoform 1 [Homo sapiens] | | | | |
| 45 | 148539840 | deleted in malignant brain tumors 1 protein | 0 | 0 | 44 | 362 |
| | | isoform a precursor [Homo sapiens] | | | | |
| 80 | 125817 | RecName: Full = Ig kappa chain V-III | 0 | 0 | 45 | 347 |
| | | region HAH; Flags: Precursor | | | | |
| 81 | 163659918 | sacsin [Homo sapiens] | 90 | 555 | 46 | 346 |
| 82 | 151301127 | dynein heavy chain 7 axonemal [Homo sapiens] | 177 | 380 | 47 | 344 |
| 83 | 297206791 | fibrous sheath-interacting protein 2 | 82 | 566 | 48 | 342 |
| | | [Homo sapiens] | | | | |
| 84 | 298351714 | RecName: Full = Ig lambda-2 chain C regions | 22 | 1470 | 49 | 338 |
| 85 | 296080693 | glucose-6-phosphate isomerase isoform | 243 | 325 | 50 | 336 |
| | | 1 [Homo sapiens] | | | | |
| 86 | 148762969 | histone-lysine N-methyltransferase | 123 | 478 | 51 | 333 |
| | | MLL2 [Homo sapiens] | | | | |
| 87 | 156766050 | protein AHNAK2 [Homo sapiens] | 111 | 499 | 52 | 330 |
| 88 | 61743954 | neuroblast differentiation-associated | 141 | 443 | 53 | 326 |
| | | protein AHNAK isoform 1 [Homo sapiens] | | | | |
| 89 | 119395750 | keratin type II cytoskeletal 1 | 98 | 542 | 54 | 320 |
| | | [Homo sapiens] | | | | |
| 90 | 113722120 | G-protein coupled receptor 98 precursor | 112 | 498 | 55 | 316 |
| | | [Homo sapiens] | | | | |
| 91 | 30520377 | CUB and zona pellucida-like domain- | 0 | 0 | 56 | 316 |
| | | containing protein 1 precursor | | | | |
| | | [Homo sapiens] | | | | |
| 92 | 257196151 | immunoglobulin-like and fibronectin | 159 | 399 | 57 | 312 |
| | | type III domain-containing protein 1 | | | | |
| | | [Homo sapiens] | | | | |
| 93 | 330688408 | nesprin-1 isoform 1 [Homo sapiens] | 0 | 0 | 58 | 311 |
| 94 | 226246554 | coiled-coil domain-containing protein | 80 | 578 | 59 | 311 |
| | | 168 [Homo sapiens] | | | | |
| 95 | 33188445 | microtubule-actin cross-linking factor 1 | 104 | 520 | 60 | 309 |
| | | isoform a [Homo sapiens] | | | | |
| 96 | 366039979 | RING finger protein 213 isoform 3 | 113 | 497 | 61 | 308 |
| | | [Homo sapiens] | | | | |
| 65 | 126032348 | E3 ubiquitin-protein ligase HERC2 | 184 | 369 | 62 | 305 |
| | | [Homo sapiens] | | | | |
| 97 | 291190787 | probable E3 ubiquitin-protein ligase | 118 | 484 | 63 | 304 |
| | | MYCBP2 [Homo sapiens] | | | | |
| 98 | 13489087 | leukocyte elastase inhibitor [Homo sapiens] | 69 | 689 | 64 | 304 |
| 99 | 4504875 | kallikrein-1 preproprotein [Homo sapiens] | 568 | 228 | 65 | 300 |
| 41 | 7669492 | glyceraldehyde-3-phosphate | 35 | 1047 | 66 | 300 |
| | | dehydrogenase [Homo sapiens] | | | | |
| 42 | 10334859 | creatine kinase U-type mitochondrial | 599 | 223 | 67 | 298 |
| | | precursor [Homo sapiens] | | | | |
| 100 | 19115954 | dynein heavy chain 5 axonemal [Homo sapiens] | 125 | 472 | 68 | 292 |
| 101 | 118918407 | nesprin-2 isoform 5 [Homo sapiens] | 0 | 0 | 69 | 291 |
| 102 | 47078295 | adenosine deaminase [Homo sapiens] | 58 | 753 | 70 | 290 |
| 103 | 223555935 | dynein heavy chain 14 axonemal | 193 | 367 | 71 | 289 |
| | | isoform 1 [Homo sapiens] | | | | |
| 104 | 13654237 | DNA-dependent protein kinase catalytic | 0 | 0 | 72 | 288 |
| | | subunit isoform 1 [Homo sapiens] | | | | |
| 105 | 110349721 | titin isoform novex-3 [Homo sapiens] | 143 | 441 | 73 | 287 |
| 106 | 119395734 | breast cancer type 2 susceptibility | 239 | 327 | 74 | 286 |
| | | protein [Homo sapiens] | | | | |
| 35 | 223942069 | enteropeptidase precursor [Homo sapiens] | 59 | 752 | 75 | 285 |
| 107 | 91199540 | dihydrolipoyl dehydrogenase | 352 | 274 | 76 | 284 |
| | | mitochondrial precursor [Homo sapiens] | | | | |
| 108 | 91718902 | histone-lysine N-methyltransferase | 181 | 375 | 77 | 283 |
| | | MLL3 [Homo sapiens] | | | | |
| 57 | 106507261 | pancreatic lipase-related protein 2 | 2178 | 122 | 78 | 281 |
| | | precursor [Homo sapiens] | | | | |
| 109 | 119703749 | hydrocephalus-inducing protein | 172 | 386 | 79 | 281 |
| | | homolog isoform a [Homo sapiens] | | | | |
| 110 | 125788 | RecName: Full = Ig kappa chain V-II | 89 | 557 | 80 | 272 |
| | | region TEW | | | | |
| 111 | 33350932 | cytoplasmic dynein 1 heavy chain 1 | 136 | 451 | 81 | 272 |
| | | [Homo sapiens] | | | | |
| 112 | 116063573 | filamin-A isoform 1 [Homo sapiens] | 399 | 262 | 82 | 271 |
| 113 | 150418007 | E3 SUMO-protein ligase RanBP2 [Homo sapiens] | 395 | 262 | 83 | 271 |
| 114 | 31657092 | ATP-binding cassette sub-family A | 183 | 370 | 84 | 271 |
| | | member 13 [Homo sapiens] | | | | |
| 115 | 150378539 | protein piccolo isoform 1 [Homo sapiens] | 127 | 467 | 85 | 270 |
| 27 | 105990532 | apolipoprotein B-100 precursor | 148 | 424 | 86 | 269 |
| | | [Homo sapiens] | | | | |
| 116 | 54607053 | translational activator GCN1 [Homo sapiens] | 354 | 272 | 87 | 266 |
| 117 | 256017163 | MAX gene-associated protein isoform 1 | 0 | 0 | 88 | 266 |
| | | [Homo sapiens] | | | | |
| 118 | 120587023 | small subunit processome component 20 | 502 | 238 | 89 | 265 |
| | | homolog [Homo sapiens] | | | | |
| 119 | 41322923 | plectin isoform 1a [Homo sapiens] | 0 | 0 | 90 | 265 |
| 120 | 226529917 | triosephosphate isomerase isoform 2 | 87 | 560 | 91 | 264 |
| | | [Homo sapiens] | | | | |
| 121 | 18375650 | tyrosine-protein phosphatase non- | 247 | 322 | 92 | 264 |
| | | receptor type 13 isoform 4 [Homo sapiens] | | | | |
| 122 | 126131099 | probable E3 ubiquitin-protein ligase | 115 | 490 | 93 | 264 |
| | | HERC1 [Homo sapiens] | | | | |
| 123 | 34577049 | bullous pemphigoid antigen 1 isoform | 121 | 479 | 94 | 263 |
| | | 1eA precursor [Homo sapiens] | | | | |
| 61 | 32967601 | ankyrin-3 isoform 1 [Homo sapiens] | 156 | 408 | 95 | 260 |
| 124 | 93102379 | low-density lipoprotein receptor-related | 314 | 289 | 96 | 259 |
| | | protein 1B precursor [Homo sapiens] | | | | |
| 125 | 118498345 | zinc finger homeobox protein 3 isoform | 345 | 277 | 97 | 258 |
| | | A [Homo sapiens] | | | | |
| 126 | 359718912 | probable E3 ubiquitin-protein ligase | 191 | 367 | 98 | 257 |
| | | C12orf51 [Homo sapiens] | | | | |
| 127 | 115527120 | nebulin isoform 3 [Homo sapiens] | 116 | 486 | 99 | 255 |
| 128 | 28559088 | laminin subunit alpha-2 isoform a | 323 | 284 | 100 | 255 |
| | | precursor [Homo sapiens] | | | | |
| 129 | 171184451 | centrosome-associated protein 350 | 317 | 287 | 101 | 252 |
| | | [Homo sapiens] | | | | |
| 130 | 221316593 | cadherin-17 precursor [Homo sapiens] | 262 | 316 | 102 | 252 |
| 131 | 150378498 | uncharacterized protein KIAA1109 | 74 | 609 | 103 | 252 |
| | | [Homo sapiens] | | | | |
| 34 | 157266292 | intestinal-type alkaline phosphatase | 153 | 413 | 104 | 251 |
| | | precursor [Homo sapiens] | | | | |
| 132 | 332688227 | dynein heavy chain 8 axonemal [Homo sapiens] | 209 | 351 | 105 | 251 |
| 133 | 62241003 | cardiomyopathy-associated protein 5 | 120 | 479 | 106 | 249 |
| | | [Homo sapiens] | | | | |
| 134 | 114155133 | dynein heavy chain 9 axonemal isoform | 155 | 412 | 107 | 249 |
| | | 2 [Homo sapiens] | | | | |
| 135 | 341913678 | PREDICTED: cadherin-23-like isoform | 197 | 365 | 108 | 247 |
| | | 1 [Homo sapiens] | | | | |
| 136 | 24415404 | midasin [Homo sapiens] | 114 | 496 | 109 | 247 |
| 137 | 88501738 | TRIO and F-actin-binding protein | 394 | 262 | 110 | 247 |
| | | isoform 6 [Homo sapiens] | | | | |
| 138 | 112799847 | ryanodine receptor 2 [Homo sapiens] | 138 | 449 | 111 | 247 |
| 139 | 122937398 | cytoplasmic dynein 2 heavy chain 1 | 0 | 0 | 112 | 246 |
| | | isoform 2 [Homo sapiens] | | | | |
| 140 | 122937514 | protein unc-13 homolog C [Homo sapiens] | 218 | 347 | 113 | 246 |
| 141 | 87196343 | PDZ domain-containing protein 2 | 316 | 289 | 114 | 245 |
| | | [Homo sapiens] | | | | |
| 142 | 256542310 | dynein heavy chain 17 axonemal [Homo sapiens] | 166 | 392 | 115 | 243 |
| 143 | 120587025 | SH3 and multiple ankyrin repeat | 441 | 250 | 116 | 241 |
| | | domains protein 1 [Homo sapiens] | | | | |
| 144 | 153792694 | baculoviral IAP repeat-containing | 189 | 368 | 117 | 240 |
| | | protein 6 [Homo sapiens] | | | | |
| 145 | 270265793 | stAR-related lipid transfer protein 9 | 164 | 394 | 118 | 240 |
| | | [Homo sapiens] | | | | |
| 146 | 38455402 | neutrophil gelatinase-associated | 83 | 565 | 119 | 240 |
| | | lipocalin precursor [Homo sapiens] | | | | |
| 147 | 5031863 | galectin-3-binding protein precursor | 91 | 551 | 120 | 239 |
| | | [Homo sapiens] | | | | |
| 148 | 22538387 | A-kinase anchor protein 9 isoform 2 | 201 | 362 | 121 | 239 |
| | | [Homo sapiens] | | | | |
| 149 | 257743023 | nebulin isoform 1 [Homo sapiens] | 0 | 0 | 122 | 239 |
| 150 | 55743098 | collagen alpha-3(VI) chain isoform 1 | 214 | 349 | 123 | 239 |
| | | precursor [Homo sapiens] | | | | |
| 151 | 306922386 | adenomatous polyposis coli protein | 0 | 0 | 124 | 237 |
| | | isoform a [Homo sapiens] | | | | |
| 152 | 295986608 | immunoglobulin lambda-like | 30 | 1193 | 125 | 236 |
| | | polypeptide 5 isoform 1 [Homo sapiens] | | | | |
| 153 | 121047 | RecName: Full = Ig gamma-4 chain C region | 102 | 528 | 126 | 234 |
| 154 | 169658378 | trinucleotide repeat-containing gene 18 | 393 | 263 | 127 | 233 |
| | | protein [Homo sapiens] | | | | |
| 155 | 149363685 | uncharacterized protein KIAA0947 | 901 | 186 | 128 | 232 |
| | | [Homo sapiens] | | | | |
| 156 | 113204617 | ryanodine receptor 1 isoform 2 [Homo sapiens] | 0 | 0 | 129 | 231 |
| 157 | 54607139 | vacuolar protein sorting-associated | 145 | 429 | 130 | 227 |
| | | protein 13D isoform 1 [Homo sapiens] | | | | |
| 158 | 4501901 | aminoacylase-1 isoform a [Homo sapiens] | 0 | 0 | 131 | 225 |
| 159 | 1730075 | RecName: Full = Ig kappa chain V-IV | 103 | 523 | 132 | 224 |
| | | region Len | | | | |
| 160 | 38788274 | nucleosome-remodeling factor subunit | 380 | 265 | 133 | 223 |
| | | BPTF isoform 1 [Homo sapiens] | | | | |
| 161 | 119120894 | dmX-like protein 2 isoform 2 [Homo sapiens] | 483 | 243 | 134 | 223 |
| 162 | 93141047 | collagen alpha-1(XII) chain long | 558 | 229 | 135 | 222 |
| | | isoform precursor [Homo sapiens] | | | | |
| 163 | 14790190 | msx2-interacting protein [Homo sapiens] | 285 | 306 | 136 | 221 |
| 164 | 194353966 | dynein heavy chain 6 axonemal [Homo sapiens] | 299 | 300 | 137 | 220 |
| 165 | 197313748 | histone-lysine N-methyltransferase | 246 | 323 | 138 | 220 |
| | | SETD2 [Homo sapiens] | | | | |
| 166 | 4502961 | collagen alpha-1(VII) chain precursor | 126 | 468 | 139 | 219 |
| | | [Homo sapiens] | | | | |
| 167 | 331284180 | nuclear receptor corepressor 2 isoform 3 | 0 | 0 | 140 | 219 |
| | | [Homo sapiens] | | | | |
| 168 | 165932370 | protocadherin Fat 4 precursor [Homo sapiens] | 206 | 356 | 141 | 219 |
| 169 | 198442844 | dynein heavy chain 10 axonemal [Homo sapiens] | 142 | 443 | 142 | 218 |
| 170 | 87196339 | collagen alpha-1(VI) chain precursor | 911 | 185 | 143 | 217 |
| | | [Homo sapiens] | | | | |
| 171 | 223633988 | uncharacterized protein KIAA1671 | 298 | 300 | 144 | 217 |
| | | [Homo sapiens] | | | | |
| 172 | 16933557 | protocadherin-16 precursor [Homo sapiens] | 327 | 283 | 145 | 217 |
| 173 | 222144249 | dynein heavy chain domain-containing | 263 | 315 | 146 | 216 |
| | | protein 1 isoform 1 [Homo sapiens] | | | | |
| 174 | 66347828 | vacuolar protein sorting-associated | 240 | 326 | 147 | 216 |
| | | protein 13C isoform 2A [Homo sapiens] | | | | |
| 175 | 113722133 | probable helicase senataxin [Homo sapiens] | 963 | 181 | 148 | 216 |
| 176 | 126012573 | low-density lipoprotein receptor-related | 215 | 349 | 149 | 215 |
| | | protein 2 precursor [Homo sapiens] | | | | |
| 177 | 118498337 | E3 ubiquitin-protein ligase HECTD1 | 296 | 301 | 150 | 215 |
| | | [Homo sapiens] | | | | |
| 178 | 4505847 | phospholipase A2 precursor [Homo sapiens] | 351 | 274 | 151 | 214 |
| 179 | 126116589 | fibrocystin-L precursor [Homo sapiens] | 280 | 307 | 152 | 214 |
| 180 | 169177000 | PREDICTED: LOW QUALITY | 157 | 406 | 153 | 214 |
| | | PROTEIN: hemicentin-2 [Homo sapiens] | | | | |
| 181 | 31563330 | A-kinase anchor protein 13 isoform 1 | 304 | 293 | 154 | 213 |
| | | [Homo sapiens] | | | | |
| 182 | 79749430 | FRAS1-related extracellular matrix | 198 | 364 | 155 | 213 |
| | | protein 2 precursor [Homo sapiens] | | | | |
| 183 | 91208420 | protein bassoon [Homo sapiens] | 137 | 450 | 156 | 212 |
| 184 | 20336205 | transcriptional regulator ATRX isoform | 0 | 0 | 157 | 211 |
| | | 2 [Homo sapiens] | | | | |
| 185 | 81295809 | pericentrin [Homo sapiens] | 135 | 451 | 158 | 211 |
| 186 | 45545421 | ectonucleotide pyrophosphatase/ | 133 | 453 | 159 | 211 |
| | | phosphodiesterase family member 7 | | | | |
| | | precursor [Homo sapiens] | | | | |
| 187 | 296011010 | protein FAM208B [Homo sapiens] | 373 | 267 | 160 | 210 |
| 188 | 82659109 | E3 ubiquitin-protein ligase UBR4 | 128 | 467 | 161 | 210 |
| | | [Homo sapiens] | | | | |
| 189 | 169178458 | PREDICTED: LOW QUALITY | 170 | 390 | 162 | 209 |
| | | PROTEIN: hemicentin-2 [Homo sapiens] | | | | |
| 190 | 116805322 | filamin-C isoform a [Homo sapiens] | 0 | 0 | 163 | 209 |
| 191 | 4557793 | neurofibromin isoform 2 [Homo sapiens] | 0 | 0 | 164 | 209 |
| 192 | 45827701 | protein dopey-2 [Homo sapiens] | 548 | 231 | 165 | 209 |
| 193 | 149158690 | protein PRRC2A [Homo sapiens] | 916 | 185 | 166 | 208 |
| 194 | 35493701 | vacuolar protein sorting-associated | 253 | 320 | 167 | 207 |
| | | protein 13B isoform 1 [Homo sapiens] | | | | |
| 195 | 15147337 | E3 ubiquitin-protein ligase UBR5 | 163 | 395 | 168 | 207 |
| | | [Homo sapiens] | | | | |
| 196 | 139948432 | matrix-remodeling-associated protein 5 | 217 | 347 | 169 | 206 |
| | | precursor [Homo sapiens] | | | | |
| 197 | 87299628 | biorientation of chromosomes in cell | 213 | 350 | 170 | 206 |
| | | division protein 1-like [Homo sapiens] | | | | |
| 198 | 89363017 | collagen alpha-2(V) chain preproprotein | 744 | 200 | 171 | 206 |
| | | [Homo sapiens] | | | | |
| 199 | 126131102 | fibrocystin isoform 1 precursor [Homo sapiens] | 223 | 342 | 172 | 205 |
| 200 | 61676188 | E3 ubiquitin-protein ligase HUWE1 | 292 | 303 | 173 | 204 |
| | | [Homo sapiens] | | | | |
| 201 | 55770834 | centromere protein F [Homo sapiens] | 174 | 384 | 174 | 204 |
| 202 | 157266317 | serine/threonine-protein kinase ATR | 402 | 261 | 175 | 204 |
| | | [Homo sapiens] | | | | |
| 203 | 105990541 | retinal-specific ATP-binding cassette | 863 | 189 | 176 | 204 |
| | | transporter [Homo sapiens] | | | | |
| 204 | 51479173 | dynein heavy chain 11 axonemal | 196 | 366 | 177 | 204 |
| | | [Homo sapiens] | | | | |
| 205 | 56550039 | histone-lysine N-methyltransferase MLL | 168 | 390 | 178 | 204 |
| | | isoform 2 precursor [Homo sapiens] | | | | |
| 206 | 170296790 | trypsin-3 isoform 1 preproprotein | 567 | 228 | 179 | 203 |
| | | [Homo sapiens] | | | | |
| 207 | 51317366 | myosin-XVIIIb [Homo sapiens] | 319 | 286 | 180 | 202 |
| 208 | 47717123 | intersectin-1 isoform ITSN-1 [Homo sapiens] | 462 | 247 | 181 | 202 |
| 209 | 4502443 | bullous pemphigoid antigen 1 isoform | 0 | 0 | 182 | 202 |
| | | 1e precursor [Homo sapiens] | | | | |
| 210 | 62243658 | serine/threonine-protein kinase SMG1 | 219 | 346 | 183 | 202 |
| | | [Homo sapiens] | | | | |
| 211 | 66346672 | vacuolar protein sorting-associated | 0 | 0 | 184 | 202 |
| | | protein 13A isoform C [Homo sapiens] | | | | |
| 212 | 111118976 | collagen alpha-1(II) chain isoform 1 | 369 | 267 | 185 | 201 |
| | | precursor [Homo sapiens] | | | | |
| 213 | 121583483 | 1-phosphatidylinositol-3-phosphate 5- | 312 | 290 | 186 | 201 |
| | | kinase isoform 2 [Homo sapiens] | | | | |
| 214 | 75677365 | dynein heavy chain 2 axonemal [Homo sapiens] | 282 | 306 | 187 | 200 |
| 215 | 27436938 | reelin isoform a precursor [Homo sapiens] | 0 | 0 | 188 | 200 |
| 216 | 21264602 | laminin subunit alpha-5 precursor | 457 | 247 | 189 | 200 |
| | | [Homo sapiens] | | | | |
| 217 | 257467639 | uncharacterized protein KIAA0889 | 1244 | 163 | 190 | 200 |
| | | isoform 1 [Homo sapiens] | | | | |
| 218 | 110349772 | collagen alpha-1(I) chain preproprotein | 492 | 240 | 192 | 199 |
| | | [Homo sapiens] | | | | |
| 219 | 119372317 | xin actin-binding repeat-containing | 194 | 366 | 193 | 199 |
| | | protein 2 isoform 1 [Homo sapiens] | | | | |
| 220 | 134268640 | alpha-tectorin precursor [Homo sapiens] | 404 | 260 | 194 | 199 |
| 19 | 116284392 | mucin-2 precursor [Homo sapiens] | 75 | 600 | 195 | 199 |
| 221 | 92110053 | CUB and sushi domain-containing | 274 | 310 | 196 | 198 |
| | | protein 2 [Homo sapiens] | | | | |
| 222 | 291167749 | zinc finger homeobox protein 4 [Homo sapiens] | 203 | 359 | 198 | 198 |
| 223 | 223029410 | talin-1 [Homo sapiens] | 281 | 307 | 200 | 197 |
| 224 | 52426735 | ankyrin-2 isoform 1 [Homo sapiens] | 160 | 398 | 201 | 196 |
| 225 | 148746189 | multiple PDZ domain protein [Homo sapiens] | 396 | 262 | 202 | 196 |
| 226 | 109633039 | receptor-type tyrosine-protein phosphatase F | 709 | 205 | 204 | 196 |
| | | isoform 2 precursor [Homo sapiens] | | | | |
| 227 | 7656967 | cadherin EGF LAG seven-pass G-type | 560 | 229 | 205 | 196 |
| | | receptor 1 precursor [Homo sapiens] | | | | |
| 228 | 239735519 | myotubularin-related protein 5 [Homo sapiens] | 342 | 278 | 206 | 196 |
| 229 | 45439359 | triple functional domain protein | 268 | 313 | 207 | 195 |
| | | [Homo sapiens] | | | | |
| 230 | 41152086 | serpin B6 [Homo sapiens] | 55 | 775 | 208 | 195 |
| 231 | 256000767 | extracellular matrix protein FRAS1 | 470 | 245 | 209 | 195 |
| | | isoform 1 precursor [Homo sapiens] | | | | |
| 232 | 19923586 | histone-lysine N-methyltransferase H3 | 300 | 300 | 210 | 195 |
| | | lysine-36 and H4 lysine-20 specific | | | | |
| | | isoform b [Homo sapiens] | | | | |
| 233 | 118572613 | serine/arginine repetitive matrix protein | 465 | 246 | 211 | 194 |
| | | 2 [Homo sapiens] | | | | |
| 234 | 153792012 | DNA polymerase zeta catalytic subunit | 278 | 308 | 213 | 193 |
| | | [Homo sapiens] | | | | |
| 235 | 149192855 | protein PRRC2B [Homo sapiens] | 340 | 278 | 214 | 193 |
| 236 | 67782321 | spectrin beta chain erythrocyte isoform | 453 | 248 | 215 | 192 |
| | | a [Homo sapiens] | | | | |
| 237 | 126116596 | abnormal spindle-like microcephaly- | 250 | 322 | 216 | 192 |
| | | associated protein isoform 1 [Homo sapiens] | | | | |
| 238 | 242332527 | hypothetical protein LOC65250 [Homo sapiens] | 353 | 272 | 217 | 192 |
| 239 | 149589008 | xaa-Pro dipeptidase isoform 1 [Homo sapiens] | 543 | 232 | 218 | 192 |
| 240 | 38788416 | laminin subunit alpha-1 precursor | 341 | 278 | 219 | 191 |
| | | [Homo sapiens] | | | | |
| 241 | 150418009 | transforming acidic coiled-coil-containing | 424 | 254 | 220 | 191 |
| | | protein 2 isoform a [Homo sapiens] | | | | |
| 242 | 4502951 | collagen alpha-1(III) chain preproprotein | 613 | 221 | 222 | 191 |
| | | [Homo sapiens] | | | | |
| 243 | 365192532 | myosin-10 isoform 1 [Homo sapiens] | 408 | 259 | 223 | 191 |
| 244 | 16445436 | bromodomain and WD repeat-containing | 495 | 240 | 224 | 191 |
| | | protein 1 isoform A [Homo sapiens] | | | | |
| 245 | 110349786 | Alstrom syndrome protein 1 [Homo sapiens] | 131 | 454 | 225 | 190 |
| 246 | 207452735 | epiplakin [Homo sapiens] | 412 | 257 | 226 | 190 |
| 247 | 153945846 | inositol 1 4 5-trisphosphate receptor | 307 | 293 | 228 | 189 |
| | | type 3 [Homo sapiens] | | | | |
| 248 | 221316747 | exophilin-5 [Homo sapiens] | 419 | 256 | 229 | 188 |
| 249 | 260064009 | ubiquitin carboxyl-terminal hydrolase 24 | 273 | 310 | 230 | 188 |
| | | [Homo sapiens] | | | | |
| 250 | 310114187 | PREDICTED: ankyrin repeat domain- | 468 | 245 | 231 | 188 |
| | | containing protein 36A [Homo sapiens] | | | | |
| 251 | 66346693 | protocadherin Fat 1 precursor | 185 | 369 | 232 | 188 |
| | | [Homo sapiens] | | | | |
| 252 | 188536004 | zinc finger protein 469 [Homo sapiens] | 122 | 479 | 233 | 188 |
| 253 | 157785645 | striated muscle preferentially expressed | 256 | 318 | 234 | 187 |
| | | protein kinase isoform 1 [Homo sapiens] | | | | |
| 254 | 38045910 | laminin subunit alpha-3 isoform 1 | 187 | 369 | 235 | 187 |
| | | precursor [Homo sapiens] | | | | |
| 255 | 26080431 | ATPase family AAA domain-containing | 360 | 270 | 236 | 187 |
| | | protein 5 [Homo sapiens] | | | | |
| 256 | 4503355 | dedicator of cytokinesis protein 1 | 413 | 257 | 238 | 186 |
| | | [Homo sapiens] | | | | |
| 257 | 259013213 | CUB and sushi domain-containing | 295 | 301 | 239 | 186 |
| | | protein 1 precursor [Homo sapiens] | | | | |
| 258 | 148886654 | sushi von Willebrand factor type A | 248 | 322 | 240 | 186 |
| | | EGF and pentraxin domain-containing | | | | |
| | | protein 1 precursor [Homo sapiens] | | | | |
| 259 | 54292123 | lysosomal-trafficking regulator | 228 | 336 | 241 | 186 |
| | | [Homo sapiens] | | | | |
| 260 | 110735435 | collagen alpha-3(V) chain preproprotein | 265 | 314 | 242 | 185 |
| | | [Homo sapiens] | | | | |
| 261 | 122891870 | melanoma inhibitory activity protein 3 | 421 | 256 | 243 | 185 |
| | | precursor [Homo sapiens] | | | | |
| 262 | 21264565 | AT-rich interactive domain-containing | 686 | 208 | 244 | 185 |
| | | protein 1A isoform a [Homo sapiens] | | | | |
| 263 | 38202205 | zinc finger FYVE domain-containing | 407 | 259 | 245 | 184 |
| | | protein 26 [Homo sapiens] | | | | |
| 264 | 13787217 | protocadherin Fat 2 precursor [Homo sapiens] | 241 | 326 | 246 | 184 |
| 265 | 24308169 | dynein heavy chain 3 axonemal [Homo sapiens] | 212 | 350 | 248 | 183 |
| 266 | 62177127 | myosin-XVI isoform 2 [Homo sapiens] | 506 | 237 | 249 | 183 |
| 267 | 224028289 | tetratricopeptide repeat protein 28 | 222 | 342 | 250 | 183 |
| | | [Homo sapiens] | | | | |
| 268 | 126723564 | pecanex-like protein 1 [Homo sapiens] | 382 | 265 | 251 | 183 |
| 269 | 12667788 | myosin-9 [Homo sapiens] | 227 | 337 | 252 | 183 |
| 270 | 126012571 | basement membrane-specific heparan | 186 | 369 | 253 | 182 |
| | | sulfate proteoglycan core protein | | | | |
| | | precursor [Homo sapiens] | | | | |
| 271 | 5031587 | adenomatous polyposis coli protein 2 | 397 | 262 | 255 | 181 |
| | | [Homo sapiens] | | | | |
| 272 | 92091572 | dedicator of cytokinesis protein 4 | 531 | 233 | 256 | 181 |
| | | [Homo sapiens] | | | | |
| 273 | 10863903 | probable E3 ubiquitin-protein ligase | 500 | 239 | 258 | 181 |
| | | TRIP 12 [Homo sapiens] | | | | |
| 274 | 90903231 | huntingtin [Homo sapiens] | 326 | 283 | 259 | 180 |
| 275 | 156104874 | envoplakin [Homo sapiens] | 315 | 289 | 263 | 179 |
| 276 | 73747881 | zinc finger ZZ-type and EF-hand | 308 | 293 | 264 | 179 |
| | | domain-containing protein 1 [Homo sapiens] | | | | |
| 277 | 150170699 | kinesin-like protein KIF26A [Homo sapiens] | 444 | 249 | 265 | 179 |
| 278 | 13325064 | cadherin EGF LAG seven-pass G-type | 286 | 305 | 267 | 178 |
| | | receptor 2 precursor [Homo sapiens] | | | | |
| 279 | 281485550 | fibrillin-1 precursor [Homo sapiens] | 632 | 217 | 268 | 178 |
| 44 | 110618248 | cadherin-related family member 5 | 107 | 511 | 271 | 177 |
| | | isoform 1 precursor [Homo sapiens] | | | | |
| 280 | 54873613 | agrin precursor [Homo sapiens] | 368 | 267 | 272 | 177 |
| 281 | 169790825 | teneurin-4 [Homo sapiens] | 313 | 289 | 273 | 177 |
| 282 | 150456444 | protein unc-79 homolog [Homo sapiens] | 329 | 282 | 274 | 177 |
| 283 | 148762940 | protein Daple [Homo sapiens] | 724 | 202 | 275 | 177 |
| 284 | 119964726 | cation-independent mannose-6-phosphate | 236 | 329 | 276 | 177 |
| | | receptor precursor [Homo sapiens] | | | | |
| 285 | 154354990 | ankyrin repeat domain-containing | 365 | 269 | 277 | 176 |
| | | protein 26 isoform 1 [Homo sapiens] | | | | |
| 286 | 62422577 | neurobeachin isoform 1 [Homo sapiens] | 410 | 258 | 279 | 176 |
| 287 | 148886692 | protocadherin Fat 3 precursor [Homo sapiens] | 290 | 303 | 281 | 175 |
| 14 | 29725633 | lithostathine-1-alpha precursor | 71 | 643 | 282 | 174 |
| | | [Homo sapiens] | | | | |
| 288 | 224458301 | protein FAM186A [Homo sapiens] | 696 | 207 | 284 | 174 |
| 289 | 7662046 | histone-lysine N-methyltransferase | 372 | 267 | 285 | 174 |
| | | MLL4 [Homo sapiens] | | | | |
| 290 | 95147335 | inositol 1 4 5-trisphosphate receptor | 288 | 304 | 286 | 174 |
| | | type 2 [Homo sapiens] | | | | |
| 291 | 54112403 | chromodomain-helicase-DNA-binding | 356 | 272 | 287 | 173 |
| | | protein 7 [Homo sapiens] | | | | |
| 292 | 191252801 | WD repeat- and FYVE domain- | 226 | 339 | 288 | 173 |
| | | containing protein 4 [Homo sapiens] | | | | |
| 24 | 66932947 | alpha-2-macroglobulin precursor | 32 | 1169 | 290 | 173 |
| | | [Homo sapiens] | | | | |
| 293 | 89276751 | collagen alpha-1(V) chain preproprotein | 336 | 280 | 291 | 173 |
| | | [Homo sapiens] | | | | |
| 294 | 255003833 | centrosomal protein of 192 kDa [Homo sapiens] | 325 | 283 | 294 | 172 |
| 295 | 50659080 | alpha-1-antichymotrypsin precursor | 23 | 1465 | 295 | 172 |
| | | [Homo sapiens] | | | | |
| 296 | 183583553 | collagen alpha-5(VI) chain precursor | 192 | 367 | 296 | 171 |
| | | [Homo sapiens] | | | | |
| 297 | 41054864 | regulating synaptic membrane exocytosis | 423 | 255 | 298 | 171 |
| | | protein 1 isoform 1 [Homo sapiens] | | | | |
| 298 | 301172750 | mucin-5B precursor [Homo sapiens] | 93 | 549 | 301 | 171 |
| 299 | 114842389 | myosin-7B [Homo sapiens] | 357 | 271 | 302 | 171 |
| 300 | 197927452 | dynein heavy chain 1 axonemal [Homo sapiens] | 165 | 393 | 305 | 170 |
| 301 | 110611228 | utrophin [Homo sapiens] | 208 | 353 | 306 | 170 |
| 302 | 55749742 | HEAT repeat-containing protein 5B | 302 | 297 | 307 | 170 |
| | | [Homo sapiens] | | | | |
| 303 | 117606355 | protein furry homolog [Homo sapiens] | 271 | 311 | 308 | 170 |
| 304 | 282165704 | chromodomain-helicase-DNA-binding | 237 | 329 | 310 | 169 |
| | | protein 8 isoform 1 [Homo sapiens] | | | | |
| 305 | 306922394 | zinc finger homeobox protein 2 [Homo sapiens] | 249 | 322 | 314 | 168 |
| 306 | 95147555 | microtubule-associated protein 1A | 272 | 310 | 316 | 168 |
| | | [Homo sapiens] | | | | |
| 307 | 359385708 | uncharacterized protein C10orf92 | 486 | 242 | 317 | 168 |
| | | [Homo sapiens] | | | | |
| 308 | 126012562 | prolow-density lipoprotein receptor- | 348 | 275 | 318 | 168 |
| | | related protein 1 precursor [Homo sapiens] | | | | |
| 309 | 332634937 | myomegalin isoform 9 [Homo sapiens] | 426 | 254 | 319 | 168 |
| 310 | 118402590 | myosin-XV [Homo sapiens] | 230 | 334 | 321 | 168 |
| 311 | 156938343 | talin-2 [Homo sapiens] | 294 | 302 | 323 | 167 |
| 312 | 126091152 | cubilin precursor [Homo sapiens] | 403 | 261 | 325 | 167 |
| 313 | 219842266 | usherin isoform B [Homo sapiens] | 277 | 308 | 326 | 167 |
| 314 | 57222563 | cytoskeleton-associated protein 5 | 409 | 259 | 330 | 167 |
| | | isoform b [Homo sapiens] | | | | |
| 315 | 148596944 | C2 domain-containing protein 3 [Homo sapiens] | 610 | 221 | 332 | 167 |
| 316 | 126032338 | ryanodine receptor 3 isoform 1 [Homo sapiens] | 149 | 424 | 333 | 166 |
| 317 | 87116683 | zinc finger C3H1 domain-containing | 671 | 211 | 336 | 165 |
| | | protein [Homo sapiens] | | | | |
| 318 | 56676397 | ankyrin repeat domain-containing | 676 | 209 | 337 | 165 |
| | | protein 11 [Homo sapiens] | | | | |
| 319 | 109255228 | centrosomal protein of 170 kDa isoform | 370 | 267 | 338 | 165 |
| | | alpha [Homo sapiens] | | | | |
| 320 | 122937512 | myosin-VIIb [Homo sapiens] | 416 | 257 | 340 | 164 |
| 321 | 188528648 | tenascin-X isoform 1 precursor [Homo sapiens] | 330 | 281 | 341 | 164 |
| 322 | 221219020 | WD repeat-containing protein 87 | 640 | 215 | 342 | 164 |
| | | [Homo sapiens] | | | | |
| 323 | 49640009 | E3 ubiquitin-protein ligase TTC3 | 371 | 267 | 344 | 164 |
| | | [Homo sapiens] | | | | |
| 324 | 109637791 | transcription factor TFIIIB component | 267 | 314 | 345 | 164 |
| | | B″ homolog [Homo sapiens] | | | | |
| 325 | 37620163 | ANKHD1-EIF4EBP3 protein [Homo sapiens] | 428 | 254 | 346 | 164 |
| 326 | 21626468 | zinc finger protein 638 isoform 1 | 692 | 208 | 347 | 164 |
| | | [Homo sapiens] | | | | |
| 327 | 116256356 | collagen alpha-4(IV) chain precursor | 732 | 201 | 352 | 163 |
| | | [Homo sapiens] | | | | |
| 328 | 310110158 | PREDICTED: otogelin isoform 1 [Homo sapiens] | 739 | 201 | 353 | 163 |
| 329 | 71061468 | centromere-associated protein E | 238 | 328 | 356 | 162 |
| | | [Homo sapiens] | | | | |
| 330 | 123853 | RecName: Full = Ig heavy chain V-III | 139 | 448 | 357 | 162 |
| | | region POM | | | | |
| 331 | 115430237 | spectrin beta chain brain 3 isoform | 242 | 325 | 358 | 162 |
| | | sigma1 [Homo sapiens] | | | | |
| 332 | 145309304 | cadherin EGF LAG seven-pass G-type | 235 | 330 | 360 | 162 |
| | | receptor 3 precursor [Homo sapiens] | | | | |
| 333 | 144226847 | obscurin-like protein 1 isoform 1 | 513 | 235 | 362 | 161 |
| | | precursor [Homo sapiens] | | | | |
| 334 | 40217847 | U5 small nuclear ribonucleoprotein 200 | 386 | 264 | 363 | 161 |
| | | kDa helicase [Homo sapiens] | | | | |
| 335 | 93352554 | probable G-protein coupled receptor 179 | 629 | 218 | 365 | 161 |
| | | precursor [Homo sapiens] | | | | |
| 336 | 239050813 | lipoxygenase homology domain-containing | 259 | 316 | 366 | 161 |
| | | protein 1 isoform 1 [Homo sapiens] | | | | |
| 337 | 21359935 | Down syndrome cell adhesion molecule- | 668 | 211 | 367 | 161 |
| | | like protein 1 [Homo sapiens] | | | | |
| 338 | 40805823 | collagen alpha-1(XXII) chain precursor | 387 | 264 | 369 | 161 |
| | | [Homo sapiens] | | | | |
| 339 | 19913408 | DNA topoisomerase 2-beta [Homo sapiens] | 328 | 282 | 372 | 160 |
| 340 | 203098098 | protein Shroom3 [Homo sapiens] | 347 | 275 | 374 | 160 |
| 341 | 57232740 | N-acetylated-alpha-linked acidic | 73 | 612 | 375 | 160 |
| | | dipeptidase-like protein [Homo sapiens] | | | | |
| 342 | 30089962 | serine/threonine-protein kinase MRCK | 587 | 225 | 376 | 159 |
| | | alpha isoform B [Homo sapiens] | | | | |
| 48 | 4502517 | carbonic anhydrase 1 [Homo sapiens] | 670 | 211 | 377 | 159 |
| 343 | 237681119 | breast cancer type 1 susceptibility | 636 | 216 | 378 | 159 |
| | | protein isoform 2 [Homo sapiens] | | | | |
| 344 | 112421122 | dnaJ homolog subfamily C member 13 | 566 | 228 | 381 | 159 |
| | | [Homo sapiens] | | | | |
| 345 | 4507691 | transformation/transcription domain- | 244 | 324 | 382 | 159 |
| | | associated protein isoform 2 [Homo sapiens] | | | | |
| 346 | 89191868 | von Willebrand factor preproprotein | 438 | 250 | 385 | 158 |
| | | [Homo sapiens] | | | | |
| 347 | 125792 | RecName: Full = Ig kappa chain V-II | 140 | 446 | 387 | 157 |
| | | region RPMI 6410; Flags: Precursor | | | | |
| 348 | 29244924 | chromodomain-helicase-DNA-binding | 324 | 284 | 389 | 157 |
| | | protein 6 [Homo sapiens] | | | | |
| 349 | 104487006 | receptor-type tyrosine-protein phosphatase S | 750 | 200 | 391 | 157 |
| | | isoform 1 precursor [Homo sapiens] | | | | |
| 350 | 48762934 | collagen alpha-2(I) chain precursor | 224 | 339 | 392 | 157 |
| | | [Homo sapiens] | | | | |
| 351 | 38679967 | acetyl-CoA carboxylase 1 isoform 2 | 461 | 247 | 393 | 157 |
| | | [Homo sapiens] | | | | |
| 352 | 34740331 | otoferlin isoform a [Homo sapiens] | 635 | 217 | 395 | 156 |
| 353 | 134031945 | SCO-spondin precursor [Homo sapiens] | 332 | 280 | 396 | 156 |
| 354 | 71361682 | nuclear mitotic apparatus protein 1 | 216 | 347 | 397 | 156 |
| | | [Homo sapiens] | | | | |
| 355 | 71902540 | serine-protein kinase ATM [Homo sapiens] | 335 | 280 | 398 | 156 |
| 356 | 140560919 | myomesin-1 isoform a [Homo sapiens] | 293 | 303 | 399 | 156 |
| 357 | 150010558 | myosin-15 precursor [Homo sapiens] | 565 | 228 | 403 | 155 |
| 358 | 148536825 | collagen alpha-1(IV) chain | 497 | 239 | 404 | 155 |
| | | preproprotein [Homo sapiens] | | | | |
| 359 | 38683860 | insulin receptor substrate 2 [Homo sapiens] | 693 | 208 | 405 | 155 |
| 360 | 32481206 | lactase-phlorizin hydrolase | 43 | 909 | 406 | 154 |
| | | preproprotein [Homo sapiens] | | | | |
| 361 | 150170718 | zinc finger protein 292 [Homo sapiens] | 637 | 216 | 407 | 154 |
| 362 | 148528998 | dmX-like protein 1 [Homo sapiens] | 458 | 247 | 409 | 154 |
| 363 | 38045888 | CUB and sushi domain-containing | 283 | 306 | 410 | 154 |
| | | protein 3 isoform 1 [Homo sapiens] | | | | |
| 364 | 112734845 | collagen alpha-1(XX) chain precursor | 731 | 201 | 411 | 154 |
| | | [Homo sapiens] | | | | |
| 365 | 38093637 | Nance-Horan syndrome protein isoform | 431 | 252 | 414 | 153 |
| | | 1 [Homo sapiens] | | | | |
| 366 | 119220552 | protein sidekick-1 isoform 1 [Homo sapiens] | 398 | 262 | 415 | 153 |
| 367 | 148612838 | uncharacterized protein KIAA2026 | 375 | 266 | 416 | 152 |
| | | [Homo sapiens] | | | | |
| 368 | 95147342 | chromodomain-helicase-DNA-binding | 362 | 269 | 419 | 152 |
| | | protein 9 [Homo sapiens] | | | | |
| 369 | 110349788 | histone-lysine N-methyltransferase | 176 | 381 | 420 | 152 |
| | | ASH1L [Homo sapiens] | | | | |
| 370 | 254826809 | prematurely terminated mRNA decay | 569 | 228 | 423 | 152 |
| | | factor-like [Homo sapiens] | | | | |
| 371 | 150417973 | supervillin isoform 2 [Homo sapiens] | 434 | 251 | 425 | 151 |
| 372 | 148596992 | alpha-protein kinase 2 [Homo sapiens] | 602 | 222 | 426 | 151 |
| 373 | 209862789 | protein MICAL-3 isoform 1 [Homo sapiens] | 344 | 277 | 427 | 151 |
| 374 | 38490688 | immunoglobulin superfamily member | 279 | 307 | 430 | 151 |
| | | 10 isoform 1 precursor [Homo sapiens] | | | | |
| 375 | 134142826 | pericentriolar material 1 protein | 581 | 226 | 432 | 151 |
| | | [Homo sapiens] | | | | |
| 376 | 87578396 | microtubule-associated protein 2 | 522 | 234 | 434 | 151 |
| | | isoform 1 [Homo sapiens] | | | | |
| 377 | 134948558 | ankyrin repeat domain-containing | 728 | 202 | 435 | 150 |
| | | protein 12 isoform 1 [Homo sapiens] | | | | |
| 378 | 169658367 | BAH and coiled-coil domain-containing | 310 | 291 | 437 | 150 |
| | | protein 1 [Homo sapiens] | | | | |
| 379 | 4502337 | zinc-alpha-2-glycoprotein precursor | 50 | 836 | 438 | 150 |
| | | [Homo sapiens] | | | | |
| 380 | 119874201 | protein furry homolog-like [Homo sapiens] | 255 | 320 | 441 | 150 |
| 381 | 30794488 | kinesin-like protein KIF27 [Homo sapiens] | 401 | 261 | 442 | 150 |
| 382 | 257467648 | microtubule-associated serine/threonine- | 119 | 480 | 444 | 149 |
| | | protein kinase 4 isoform c [Homo sapiens] | | | | |
| 383 | 55956899 | keratin type I cytoskeletal 9 [Homo sapiens] | 100 | 528 | 445 | 149 |
| 384 | 58331187 | T-lymphoma invasion and metastasis- | 530 | 233 | 447 | 149 |
| | | inducing protein 2 isoform a [Homo sapiens] | | | | |
| 385 | 154350241 | brefeldin A-inhibited guanine nucleotide- | 625 | 218 | 448 | 149 |
| | | exchange protein 3 [Homo sapiens] | | | | |
| 386 | 262118282 | plexin-A1 precursor [Homo sapiens] | 690 | 208 | 451 | 148 |
| 387 | 21493045 | A-kinase anchor protein 6 [Homo sapiens] | 429 | 253 | 452 | 148 |
| 388 | 156105693 | peroxisomal proliferator-activated | 417 | 256 | 453 | 148 |
| | | receptor A-interacting complex 285 kDa | | | | |
| | | protein isoform 1 [Homo sapiens] | | | | |
| 389 | 194440727 | dynein heavy chain 12 axonemal | 199 | 363 | 456 | 148 |
| | | isoform 1 [Homo sapiens] | | | | |
| 390 | 335353804 | protein SZT2 [Homo sapiens] | 433 | 251 | 461 | 148 |
| 391 | 157738645 | plexin-A4 isoform 1 precursor [Homo sapiens] | 414 | 257 | 462 | 148 |
| 392 | 114431248 | basic helix-loop-helix domain-containing | 651 | 213 | 464 | 147 |
| | | protein KIAA2018 [Homo sapiens] | | | | |
| 393 | 371877632 | armadillo repeat-containing X-linked | 363 | 269 | 466 | 147 |
| | | protein 4 [Homo sapiens] | | | | |
| 394 | 116256354 | collagen alpha-2(IV) chain | 715 | 204 | 468 | 147 |
| | | preproprotein [Homo sapiens] | | | | |
| 395 | 145309309 | probable ubiquitin carboxyl-terminal | 322 | 284 | 469 | 147 |
| | | hydrolase FAF-X isoform 3 [Homo sapiens] | | | | |
| 396 | 47059046 | protocadherin-23 isoform 1 [Homo sapiens] | 547 | 231 | 476 | 147 |
| 397 | 115511036 | alpha-protein kinase 3 [Homo sapiens] | 746 | 200 | 478 | 146 |
| 398 | 87298937 | centriolin [Homo sapiens] | 367 | 267 | 480 | 146 |
| 399 | 267844811 | neuron navigator 1 isoform 1 [Homo sapiens] | 442 | 249 | 482 | 146 |
| 400 | 195972871 | 1-phosphatidylinositol-4 5-bisphosphate | 643 | 215 | 486 | 145 |
| | | phosphodiesterase eta-1 isoform a | | | | |
| | | [Homo sapiens] | | | | |
| 401 | 134142062 | acetyl-CoA carboxylase 2 precursor | 318 | 286 | 488 | 145 |
| | | [Homo sapiens] | | | | |
| 402 | 156104908 | myosin-6 [Homo sapiens] | 56 | 767 | 490 | 145 |
| 403 | 31563507 | GRIP and coiled-coil domain-containing | 638 | 216 | 492 | 145 |
| | | protein 2 [Homo sapiens] | | | | |
| 404 | 148233596 | lipopolysaccharide-responsive and | 605 | 222 | 495 | 144 |
| | | beige-like anchor protein isoform 2 | | | | |
| | | [Homo sapiens] | | | | |
| 405 | 262359929 | protein ELYS [Homo sapiens] | 188 | 369 | 497 | 144 |
| 406 | 154240686 | FYVE RhoGEF and PH domain- | 658 | 213 | 498 | 144 |
| | | containing protein 6 [Homo sapiens] | | | | |
| 407 | 110611226 | protein unc-13 homolog B [Homo sapiens] | 642 | 215 | 499 | 144 |
| 408 | 291190781 | leucine-rich repeat-containing protein | 749 | 200 | 500 | 144 |
| | | 16A isoform 1 [Homo sapiens] | | | | |
| 409 | 153791497 | rootletin [Homo sapiens] | 430 | 252 | 501 | 143 |
| 410 | 122937211 | proteasome-associated protein ECM29 | 710 | 205 | 503 | 143 |
| | | homolog [Homo sapiens] | | | | |
| 411 | 103472005 | antigen KI-67 isoform 1 [Homo sapiens] | 229 | 336 | 504 | 143 |
| 412 | 157738667 | FYVE and coiled-coil domain- | 563 | 228 | 507 | 143 |
| | | containing protein 1 [Homo sapiens] | | | | |
| 413 | 118600981 | probable JmjC domain-containing | 542 | 232 | 511 | 142 |
| | | histone demethylation protein 2C | | | | |
| | | isoform a [Homo sapiens] | | | | |
| 414 | 61743980 | stabilin-2 precursor [Homo sapiens] | 515 | 235 | 512 | 142 |
| 415 | 116534898 | desmoglein-2 preproprotein [Homo sapiens] | 685 | 209 | 515 | 142 |
| 416 | 115527097 | serine/threonine-protein kinase MRCK | 623 | 219 | 516 | 141 |
| | | beta [Homo sapiens] | | | | |
| 417 | 194294554 | SET-binding protein isoform a [Homo sapiens] | 425 | 254 | 517 | 141 |
| 418 | 110624781 | myosin-13 [Homo sapiens] | 364 | 269 | 520 | 140 |
| 419 | 149363642 | coiled-coil domain-containing protein | 519 | 235 | 522 | 140 |
| | | 144A [Homo sapiens] | | | | |
| 420 | 112821681 | G protein-regulated inducer of neurite | 551 | 231 | 523 | 140 |
| | | outgrowth 1 [Homo sapiens] | | | | |
| 421 | 58530840 | desmoplakin isoform I [Homo sapiens] | 305 | 293 | 525 | 140 |
| 422 | 74136549 | AT-rich interactive domain-containing | 627 | 218 | 526 | 139 |
| | | protein 5B isoform 1 [Homo sapiens] | | | | |
| 423 | 157426887 | dedicator of cytokinesis protein 6 | 538 | 232 | 527 | 139 |
| | | [Homo sapiens] | | | | |
| 424 | 71143119 | signal-induced proliferation-associated | 475 | 243 | 532 | 139 |
| | | 1-like protein 3 [Homo sapiens] | | | | |
| 425 | 94681049 | WD repeat-containing protein 96 | 507 | 237 | 534 | 139 |
| | | [Homo sapiens] | | | | |
| 426 | 146219843 | helicase SRCAP [Homo sapiens] | 350 | 274 | 538 | 139 |
| 427 | 56676335 | telomere-associated protein RIF1 | 346 | 276 | 542 | 138 |
| | | isoform 1 [Homo sapiens] | | | | |
| 428 | 53832009 | voltage-dependent T-type calcium | 630 | 218 | 543 | 138 |
| | | channel subunit alpha-1H isoform a | | | | |
| | | [Homo sapiens] | | | | |
| 429 | 148536869 | ninein isoform 2 [Homo sapiens] | 439 | 250 | 544 | 138 |
| 430 | 170016091 | teneurin-2 [Homo sapiens] | 275 | 308 | 551 | 137 |
| 431 | 183396804 | regulation of nuclear pre-mRNA domain- | 665 | 212 | 552 | 137 |
| | | containing protein 2 [Homo sapiens] | | | | |
| 432 | 205360962 | polycystin-1 isoform 2 precursor | 269 | 313 | 553 | 137 |
| | | [Homo sapiens] | | | | |
| 433 | 270133251 | amyotrophic lateral sclerosis 2 | 631 | 217 | 559 | 136 |
| | | chromosomal region candidate gene 11 | | | | |
| | | protein isoform 1 [Homo sapiens] | | | | |
| 434 | 98986453 | myosin-3 [Homo sapiens] | 77 | 586 | 560 | 136 |
| 435 | 54112429 | dedicator of cytokinesis protein 7 | 606 | 222 | 561 | 136 |
| | | [Homo sapiens] | | | | |
| 436 | 119703755 | laminin subunit beta-2 precursor | 704 | 206 | 564 | 136 |
| | | [Homo sapiens] | | | | |
| 437 | 148839466 | kalirin isoform 1 [Homo sapiens] | 254 | 320 | 566 | 136 |
| 438 | 115583670 | T-lymphoma invasion and metastasis- | 450 | 248 | 568 | 136 |
| | | inducing protein 1 [Homo sapiens] | | | | |
| 439 | 110611903 | myosin-4 [Homo sapiens] | 28 | 1297 | 570 | 135 |
| 440 | 217330594 | tubulin polyglutamylase TTLL4 [Homo sapiens] | 544 | 232 | 572 | 135 |
| 441 | 21361831 | partitioning defective 3 homolog | 730 | 202 | 576 | 135 |
| | | isoform 1 [Homo sapiens] | | | | |
| 442 | 38202209 | methyl-CpG-binding domain protein 5 | 703 | 206 | 580 | 135 |
| | | [Homo sapiens] | | | | |
| 443 | 110347463 | transcription factor HIVEP2 [Homo sapiens] | 546 | 232 | 584 | 135 |
| 444 | 111118970 | collagen alpha-2(XI) chain isoform 1 | 652 | 213 | 589 | 134 |
| | | preproprotein [Homo sapiens] | | | | |
| 445 | 190194412 | thyroid receptor-interacting protein 11 | 655 | 213 | 591 | 134 |
| | | [Homo sapiens] | | | | |
| 446 | 116006951 | polycystic kidney disease protein 1-like | 590 | 225 | 595 | 134 |
| | | 2 isoform a precursor [Homo sapiens] | | | | |
| 447 | 224451128 | protein eyes shut homolog isoform 1 | 456 | 247 | 597 | 134 |
| | | [Homo sapiens] | | | | |
| 448 | 115527062 | collagen alpha-2(VI) chain isoform 2C2 | 621 | 219 | 600 | 133 |
| | | precursor [Homo sapiens] | | | | |
| 449 | 50959205 | adenylate cyclase type 9 [Homo sapiens] | 578 | 226 | 602 | 133 |
| 450 | 21361458 | rho guanine nucleotide exchange factor | 333 | 280 | 607 | 133 |
| | | 17 [Homo sapiens] | | | | |
| 451 | 40254442 | plexin-B1 precursor [Homo sapiens] | 699 | 207 | 615 | 132 |
| 452 | 222352127 | protein sidekick-2 precursor [Homo sapiens] | 377 | 266 | 618 | 132 |
| 453 | 71565160 | structural maintenance of chromosomes | 672 | 210 | 619 | 132 |
| | | protein 1B [Homo sapiens] | | | | |
| 454 | 331284125 | E1A-binding protein p400 [Homo sapiens] | 521 | 234 | 622 | 132 |
| 455 | 134133288 | zinc finger protein 407 isoform 1 | 600 | 223 | 626 | 131 |
| | | [Homo sapiens] | | | | |
| 456 | 148806908 | fibronectin type III domain-containing | 537 | 232 | 629 | 131 |
| | | protein 1 precursor [Homo sapiens] | | | | |
| 457 | 153945790 | myosin-8 [Homo sapiens] | 34 | 1053 | 631 | 131 |
| 458 | 170016061 | spectrin beta chain brain 4 [Homo sapiens] | 152 | 416 | 633 | 131 |
| 459 | 19923084 | polycystic kidney disease protein 1-like | 385 | 264 | 640 | 130 |
| | | 1 [Homo sapiens] | | | | |
| 460 | 51339291 | sterile alpha motif domain-containing | 586 | 225 | 644 | 130 |
| | | protein 9-like [Homo sapiens] | | | | |
| 461 | 93102424 | protein FAM179B [Homo sapiens] | 525 | 234 | 652 | 129 |
| 462 | 55743096 | collagen alpha-1(XIV) chain precursor | 473 | 244 | 657 | 129 |
| | | [Homo sapiens] | | | | |
| 463 | 283837842 | protein unc-13 homolog A [Homo sapiens] | 550 | 231 | 661 | 129 |
| 464 | 47078218 | ATP-binding cassette sub-family A | 498 | 239 | 666 | 129 |
| | | member 2 isoform b [Homo sapiens] | | | | |
| 465 | 51599156 | chromodomain-helicase-DNA-binding | 306 | 293 | 667 | 129 |
| | | protein 4 [Homo sapiens] | | | | |
| 466 | 310119144 | PREDICTED: rootletin [Homo sapiens] | 388 | 264 | 672 | 128 |
| 467 | 119943102 | CREB-binding protein isoform b | 454 | 247 | 683 | 128 |
| | | [Homo sapiens] | | | | |
| 468 | 332801082 | citron Rho-interacting kinase isoform 1 | 366 | 269 | 685 | 127 |
| | | [Homo sapiens] | | | | |
| 469 | 93277088 | mediator of RNA polymerase II transcription | 738 | 201 | 690 | 127 |
| | | subunit 12-like protein [Homo sapiens] | | | | |
| 470 | 120587019 | zinc finger protein 318 [Homo sapiens] | 427 | 254 | 693 | 127 |
| 471 | 156139122 | methylcytosine dioxygenase TET1 | 303 | 296 | 697 | 127 |
| | | [Homo sapiens] | | | | |
| 472 | 150417986 | brefeldin A-inhibited guanine nucleotide- | 467 | 245 | 698 | 126 |
| | | exchange protein 2 [Homo sapiens] | | | | |
| 473 | 46358428 | intraflagellar transport protein 172 | 556 | 230 | 700 | 126 |
| | | homolog [Homo sapiens] | | | | |
| 474 | 100913220 | collagen alpha-1(XVI) chain precursor | 411 | 258 | 705 | 126 |
| | | [Homo sapiens] | | | | |
| 475 | 5902122 | spectrin beta chain brain 2 [Homo sapiens] | 480 | 243 | 708 | 126 |
| 476 | 149274646 | uncharacterized protein KIAA1614 | 620 | 219 | 711 | 126 |
| | | [Homo sapiens] | | | | |
| 477 | 47578105 | nipped-B-like protein isoform A [Homo sapiens] | 232 | 332 | 712 | 125 |
| 478 | 19923191 | 80 kDa MCM3-associated protein [Homo sapiens] | 459 | 247 | 713 | 125 |
| 21 | 4557871 | serotransferrin precursor [Homo sapiens] | 64 | 731 | 717 | 125 |
| 480 | 90991702 | leucine-rich repeat serine/threonine- | 503 | 238 | 726 | 124 |
| | | protein kinase 1 [Homo sapiens] | | | | |
| 481 | 122937400 | teneurin-3 [Homo sapiens] | 418 | 256 | 728 | 124 |
| 482 | 6715600 | Golgin subfamily A member 4 isoform 2 | 374 | 266 | 735 | 123 |
| | | [Homo sapiens] | | | | |
| 483 | 92859678 | snRNA-activating protein complex | 501 | 238 | 736 | 123 |
| | | subunit 4 [Homo sapiens] | | | | |
| 484 | 333440449 | CLIP-associating protein 2 isoform 1 | 526 | 234 | 743 | 123 |
| | | [Homo sapiens] | | | | |
| 485 | 120953251 | neuron navigator 3 [Homo sapiens] | 571 | 227 | 748 | 123 |
| 486 | 157952215 | receptor- type tyrosine-protein | 422 | 256 | 749 | 123 |
| | | phosphatase beta isoform a [Homo sapiens] | | | | |
| 487 | 160948599 | integrator complex subunit 1 [Homo sapiens] | 700 | 207 | 754 | 122 |
| 488 | 89142730 | collagen alpha-3(IV) chain precursor | 596 | 224 | 756 | 122 |
| | | [Homo sapiens] | | | | |
| 489 | 18105007 | CAD protein [Homo sapiens] | 684 | 209 | 760 | 122 |
| 490 | 44771211 | mediator of RNA polymerase II | 697 | 207 | 761 | 122 |
| | | transcription subunit 13-like [Homo sapiens] | | | | |
| 491 | 110347427 | ubiquitin carboxyl-terminal hydrolase 34 | 169 | 390 | 765 | 121 |
| | | [Homo sapiens] | | | | |
| 492 | 21536376 | ATP-binding cassette sub-family A | 694 | 207 | 766 | 121 |
| | | member 1 [Homo sapiens] | | | | |
| 493 | 224451124 | neurobeachin-like protein 1 [Homo sapiens] | 337 | 280 | 768 | 121 |
| 494 | 74048554 | protein CASC5 isoform 2 [Homo sapiens] | 376 | 266 | 775 | 120 |
| 495 | 21361116 | versican core protein isoform 1 | 455 | 247 | 779 | 120 |
| | | precursor [Homo sapiens] | | | | |
| 496 | 112382257 | inaD-like protein [Homo sapiens] | 718 | 204 | 780 | 120 |
| 497 | 40255272 | xin actin-binding repeat-containing | 205 | 358 | 781 | 120 |
| | | protein 1 isoform 1 [Homo sapiens] | | | | |
| 498 | 348041302 | phosphatidylinositol 4-kinase alpha | 617 | 220 | 782 | 120 |
| | | isoform 1 [Homo sapiens] | | | | |
| 499 | 231573214 | E3 ubiquitin-protein ligase listerin | 512 | 236 | 787 | 120 |
| | | [Homo sapiens] | | | | |
| 500 | 4507157 | sortilin-related receptor preproprotein | 675 | 209 | 790 | 119 |
| | | [Homo sapiens] | | | | |
| 501 | 150378463 | histone acetyltransferase KAT6A | 607 | 222 | 792 | 119 |
| | | [Homo sapiens] | | | | |
| 502 | 6912288 | CASP8-associated protein 2 [Homo sapiens] | 592 | 224 | 796 | 119 |
| 503 | 50843820 | sickle tail protein homolog isoform 1 | 616 | 220 | 800 | 119 |
| | | [Homo sapiens] | | | | |
| 504 | 33946282 | protein virilizer homolog isoform 1 | 594 | 224 | 801 | 119 |
| | | [Homo sapiens] | | | | |
| 505 | 118600961 | ral GTPase-activating protein subunit | 669 | 211 | 802 | 119 |
| | | alpha-2 [Homo sapiens] | | | | |
| 506 | 154813199 | poly [ADP-ribose] polymerase 14 [Homo sapiens] | 384 | 264 | 805 | 119 |
| 507 | 4757960 | cadherin-1 preproprotein [Homo sapiens] | 129 | 467 | 811 | 118 |
| 508 | 11968023 | zinc finger protein 106 homolog [Homo sapiens] | 557 | 230 | 818 | 118 |
| 509 | 310110100 | PREDICTED: mucin-5AC [Homo sapiens] | 200 | 363 | 829 | 117 |
| 510 | 21735548 | centrosome-associated protein CEP250 | 320 | 285 | 831 | 117 |
| | | [Homo sapiens] | | | | |
| 511 | 239582741 | FERM and PDZ domain-containing | 653 | 213 | 837 | 117 |
| | | protein 1 [Homo sapiens] | | | | |
| 512 | 197245440 | uncharacterized protein KIAA1107 | 533 | 233 | 839 | 116 |
| | | [Homo sapiens] | | | | |
| 513 | 116268127 | protein very KIND isoform a [Homo sapiens] | 476 | 243 | 844 | 116 |
| 514 | 41872631 | fatty acid synthase [Homo sapiens] | 585 | 225 | 848 | 116 |
| 515 | 124430752 | kinesin-like protein KIF26B [Homo sapiens] | 708 | 205 | 851 | 115 |
| 516 | 154354979 | unconventionnal myosin-X [Homo sapiens] | 589 | 225 | 852 | 115 |
| 517 | 115496169 | myosin-7 [Homo sapiens] | 49 | 852 | 861 | 115 |
| 518 | 148806881 | uncharacterized protein KIAA1462 | 608 | 222 | 862 | 115 |
| | | [Homo sapiens] | | | | |
| 519 | 164607133 | fer-1-like protein 5 [Homo sapiens] | 663 | 212 | 865 | 115 |
| 520 | 32313593 | olfactomedin-4 precursor [Homo sapiens] | 67 | 705 | 866 | 115 |
| 521 | 222537743 | phosphotidylinositol phosphatase | 723 | 202 | 873 | 114 |
| | | PTPRQ precursor [Homo sapiens] | | | | |
| 522 | 341915841 | PREDICTED: hypothetical protein | 597 | 224 | 875 | 114 |
| | | LOC100129543 [Homo sapiens] | | | | |
| 523 | 149944526 | putative Polycomb group protein | 727 | 202 | 876 | 114 |
| | | ASXL3 [Homo sapiens] | | | | |
| 524 | 19923790 | rab3 GTPase-activating protein non- | 646 | 214 | 880 | 114 |
| | | catalytic subunit [Homo sapiens] | | | | |
| 525 | 41393547 | neuroblastoma-amplified sequence | 471 | 244 | 884 | 114 |
| | | [Homo sapiens] | | | | |
| 526 | 178557739 | complement C4-B preproprotein [Homo sapiens] | 720 | 203 | 888 | 114 |
| 527 | 183396787 | BCL-6 corepressor isoform c [Homo sapiens] | 540 | 232 | 890 | 113 |
| 528 | 223468663 | aldo-keto reductase family 1 member | 70 | 667 | 895 | 113 |
| | | B10 [Homo sapiens] | | | | |
| 529 | 16357503 | collagen alpha-6(IV) chain isoform B | 291 | 303 | 897 | 113 |
| | | precursor [Homo sapiens] | | | | |
| 530 | 33620745 | pre-mRNA cleavage complex 2 protein | 664 | 212 | 908 | 113 |
| | | Pcf11 [Homo sapiens] | | | | |
| 531 | 134276943 | separin [Homo sapiens] | 555 | 231 | 909 | 113 |
| 532 | 194328738 | uncharacterized protein KIAA0556 | 440 | 250 | 911 | 113 |
| | | [Homo sapiens] | | | | |
| 533 | 222537754 | uncharacterized protein C3orf77 [Homo sapiens] | 472 | 244 | 921 | 112 |
| 534 | 56711286 | uncharacterized protein KIAA2022 | 721 | 203 | 935 | 111 |
| | | [Homo sapiens] | | | | |
| 535 | 54607120 | lactotransferrin isoform 1 precursor | 42 | 914 | 936 | 111 |
| | | [Homo sapiens] | | | | |
| 536 | 256773222 | uncharacterized protein C12orf35 | 516 | 235 | 940 | 111 |
| | | [Homo sapiens] | | | | |
| 537 | 31317272 | WD repeat and FYVE domain- | 266 | 314 | 944 | 111 |
| | | containing protein 3 [Homo sapiens] | | | | |
| 538 | 89111135 | multidrug resistance-associated protein | 741 | 201 | 947 | 111 |
| | | 9 [Homo sapiens] | | | | |
| 539 | 102468717 | mediator of RNA polymerase II | 603 | 222 | 959 | 110 |
| | | transcription subunit 13 [Homo sapiens] | | | | |
| 540 | 257196142 | piezo-type mechanosensitive ion | 622 | 219 | 974 | 110 |
| | | channel component 1 [Homo sapiens] | | | | |
| 541 | 38372909 | lysine-specific demethylase 3B [Homo sapiens] | 661 | 212 | 982 | 110 |
| 542 | 59891448 | rapamycin-insensitive companion of | 717 | 204 | 986 | 109 |
| | | mTOR [Homo sapiens] | | | | |
| 543 | 153946395 | tenascin precursor [Homo sapiens] | 509 | 237 | 1003 | 108 |
| 544 | 149944548 | neurobeachin-like protein 2 [Homo sapiens] | 674 | 210 | 1007 | 108 |
| 545 | 21536371 | telomerase protein component 1 [Homo sapiens] | 485 | 242 | 1016 | 108 |
| 47 | 98986445 | carcinoembryonic antigen-related cell adhesion | 36 | 996 | 1020 | 108 |
| | | molecule 5 preproprotein [Homo sapiens] | | | | |
| 546 | 150417984 | ATP-binding cassette sub-family A | 554 | 231 | 1022 | 108 |
| | | member 7 [Homo sapiens] | | | | |
| 547 | 10835063 | nucleophosmin isoform 1 [Homo sapiens] | 289 | 304 | 1036 | 106 |
| 548 | 237858799 | adenylate kinase domain-containing | 508 | 237 | 1038 | 106 |
| | | protein 1 isoform 1 [Homo sapiens] | | | | |
| 549 | 291219891 | PH domain leucine-rich repeat-containing | 559 | 229 | 1039 | 106 |
| | | protein phosphatase 1 [Homo sapiens] | | | | |
| 550 | 115334682 | SRC kinase signaling inhibitor 1 | 359 | 270 | 1043 | 106 |
| | | [Homo sapiens] | | | | |
| 551 | 162287219 | protein prune homolog 2 [Homo sapiens] | 487 | 242 | 1044 | 106 |
| 552 | 70980549 | protein RRP5 homolog [Homo sapiens] | 742 | 200 | 1045 | 106 |
| 553 | 33946327 | nuclear pore complex protein Nup214 | 618 | 220 | 1046 | 106 |
| | | [Homo sapiens] | | | | |
| 554 | 302565871 | uncharacterized protein C9orf174 | 698 | 207 | 1055 | 106 |
| | | [Homo sapiens] | | | | |
| 555 | 167857790 | alpha-1-acid glycoprotein 1 precursor | 204 | 358 | 1063 | 106 |
| | | [Homo sapiens] | | | | |
| 556 | 38348729 | uncharacterized protein C9orf93 [Homo sapiens] | 580 | 226 | 1076 | 105 |
| 557 | 50658063 | structural maintenance of chromosomes | 532 | 233 | 1083 | 105 |
| | | protein 4 [Homo sapiens] | | | | |
| 558 | 139394648 | DNA polymerase theta [Homo sapiens] | 436 | 250 | 1086 | 105 |
| 559 | 218083800 | rho GTPase-activating protein 32 | 644 | 215 | 1106 | 104 |
| | | isoform 1 [Homo sapiens] | | | | |
| 560 | 7706457 | A-kinase anchor protein 11 [Homo sapiens] | 598 | 223 | 1110 | 104 |
| 561 | 19923723 | ribosomal protein S6 kinase delta-1 | 722 | 203 | 1116 | 103 |
| | | isoform a [Homo sapiens] | | | | |
| 562 | 4502523 | voltage-dependent N-type calcium channel | 601 | 223 | 1122 | 103 |
| | | subunit alpha-1B isoform 1 [Homo sapiens] | | | | |
| 563 | 30089940 | Golgin subfamily A member 3 isoform 1 | 619 | 220 | 1130 | 103 |
| | | [Homo sapiens] | | | | |
| 564 | 122937345 | myosin-Vb [Homo sapiens] | 733 | 201 | 1132 | 103 |
| 565 | 45387958 | protein phosphatase 1 regulatory subunit | 504 | 238 | 1146 | 102 |
| | | 26 [Homo sapiens] | | | | |
| 566 | 27436873 | E3 ubiquitin-protein ligase SHPRH | 448 | 248 | 1152 | 102 |
| | | isoform b [Homo sapiens] | | | | |
| 567 | 341914961 | PREDICTED: FERM and PDZ domain- | 479 | 243 | 1159 | 102 |
| | | containing protein 3 [Homo sapiens] | | | | |
| 568 | 299829223 | coiled-coil domain-containing protein | 751 | 200 | 1167 | 101 |
| | | 141 [Homo sapiens] | | | | |
| 569 | 7662126 | signal-induced proliferation-associated | 650 | 213 | 1188 | 100 |
| | | 1-like protein 1 [Homo sapiens] | | | | |
| 570 | 150378549 | EH domain-binding protein 1-like | 584 | 225 | 1193 | 100 |
| | | protein 1 [Homo sapiens] | | | | |
| 571 | 168823435 | calpain-7-like protein [Homo sapiens] | 493 | 240 | 1212 | 100 |
| 572 | 32455273 | serine/threonine-protein kinase WNK2 | 659 | 213 | 1216 | 99 |
| | | [Homo sapiens] | | | | |
| 573 | 153945715 | myosin-Vc [Homo sapiens] | 491 | 241 | 1227 | 99 |
| 574 | 33620775 | kinectin isoform a [Homo sapiens] | 535 | 232 | 1229 | 99 |
| 575 | 341915544 | PREDICTED: LOW QUALITY | 151 | 418 | 1232 | 99 |
| | | PROTEIN: mucin-5AC [Homo sapiens] | | | | |
| 30 | 4557485 | ceruloplasmin precursor [Homo sapiens] | 338 | 279 | 1234 | 99 |
| 576 | 54792138 | probable helicase with zinc finger | 511 | 237 | 1237 | 99 |
| | | domain [Homo sapiens] | | | | |
| 577 | 63252863 | structure-specific endonuclease subunit | 660 | 213 | 1241 | 99 |
| | | SLX4 [Homo sapiens] | | | | |
| 578 | 150036262 | calcium-activated chloride channel | 252 | 321 | 1247 | 99 |
| | | regulator 4 precursor [Homo sapiens] | | | | |
| 579 | 38348727 | thyroid adenoma-associated protein | 435 | 251 | 1256 | 98 |
| | | [Homo sapiens] | | | | |
| 580 | 218505835 | membrane-associated guanylate kinase | 576 | 227 | 1264 | 98 |
| | | WW and PDZ domain-containing | | | | |
| | | protein 3 isoform 1 [Homo sapiens] | | | | |
| 581 | 178056552 | condensin complex subunit 1 [Homo sapiens] | 518 | 235 | 1286 | 97 |
| 582 | 44889475 | DENN domain-containing protein 5A | 740 | 201 | 1299 | 96 |
| | | isoform 1 [Homo sapiens] | | | | |
| 583 | 282721063 | uncharacterized protein C1orf173 | 549 | 231 | 1317 | 96 |
| | | [Homo sapiens] | | | | |
| 584 | 122891862 | DENN domain-containing protein 5B | 705 | 206 | 1335 | 95 |
| | | [Homo sapiens] | | | | |
| 585 | 112382250 | spectrin beta chain brain 1 isoform 1 | 258 | 317 | 1352 | 94 |
| | | [Homo sapiens] | | | | |
| 586 | 156119615 | myosin-IXa [Homo sapiens] | 505 | 238 | 1358 | 94 |
| 587 | 221139764 | PHD and RING finger domain- | 494 | 240 | 1367 | 94 |
| | | containing protein 1 [Homo sapiens] | | | | |
| 588 | 242246985 | clathrin heavy chain 2 isoform 1 | 654 | 213 | 1385 | 93 |
| | | [Homo sapiens] | | | | |
| 589 | 4502271 | sodium/potassium-transporting ATPase | 734 | 201 | 1413 | 92 |
| | | subunit alpha-2 proprotein [Homo sapiens] | | | | |
| 590 | 155030216 | sister chromatid cohesion protein PDS5 | 604 | 222 | 1422 | 92 |
| | | homolog A isoform 1 [Homo sapiens] | | | | |
| 591 | 47419930 | chondroitin sulfate proteoglycan 4 | 378 | 266 | 1428 | 92 |
| | | precursor [Homo sapiens] | | | | |
| 592 | 4759146 | slit homolog 2 protein precursor | 657 | 213 | 1442 | 91 |
| | | [Homo sapiens] | | | | |
| 593 | 18079216 | caskin-1 [Homo sapiens] | 562 | 228 | 1444 | 91 |
| 594 | 188528675 | slit homolog 1 protein precursor | 614 | 220 | 1492 | 90 |
| | | [Homo sapiens] | | | | |
| 595 | 281485608 | trefoil factor 3 precursor [Homo sapiens] | 678 | 209 | 1501 | 89 |
| 596 | 105990535 | coagulation factor V precursor [Homo sapiens] | 679 | 209 | 1504 | 89 |
| 597 | 57863271 | uncharacterized protein KIAA0564 | 628 | 218 | 1514 | 89 |
| | | isoform a precursor [Homo sapiens] | | | | |
| 598 | 255759952 | WD repeat-containing protein 81 | 695 | 207 | 1532 | 88 |
| | | isoform 1 [Homo sapiens] | | | | |
| 599 | 171906559 | peripheral-type benzodiazepine receptor- | 452 | 248 | 1533 | 88 |
| | | associated protein 1 isoform a [Homo sapiens] | | | | |
| 600 | 115529484 | CD 109 antigen isoform 1 precursor | 714 | 204 | 1543 | 88 |
| | | [Homo sapiens] | | | | |
| 601 | 54607035 | integrin beta-4 isoform 1 precursor | 391 | 263 | 1544 | 88 |
| | | [Homo sapiens] | | | | |
| 602 | 157426864 | zinc finger FYVE domain-containing | 634 | 217 | 1554 | 88 |
| | | protein 16 [Homo sapiens] | | | | |
| 603 | 11321571 | slit homolog 3 protein precursor | 645 | 214 | 1556 | 87 |
| | | [Homo sapiens] | | | | |
| 604 | 94400919 | WD repeat-containing protein 90 [Homo sapiens] | 520 | 234 | 1567 | 87 |
| 605 | 207028821 | RNA-binding protein 44 [Homo sapiens] | 588 | 225 | 1580 | 86 |
| 606 | 207113160 | treacle protein isoform d [Homo sapiens] | 574 | 227 | 1599 | 86 |
| 607 | 221219000 | inactive phospholipase C-like protein 2 | 706 | 206 | 1645 | 84 |
| | | isoform 1 [Homo sapiens] | | | | |
| 608 | 256600196 | rap guanine nucleotide exchange factor | 682 | 209 | 1657 | 84 |
| | | 6 isoform 1 [Homo sapiens] | | | | |
| 609 | 4557565 | DNA excision repair protein ERCC-6 | 536 | 232 | 1679 | 83 |
| | | [Homo sapiens] | | | | |
| 610 | 308736994 | NACHT and WD repeat domain- | 496 | 239 | 1703 | 83 |
| | | containing protein 1 [Homo sapiens] | | | | |
| 611 | 303304991 | centrosomal protein of 152 kDa isoform | 361 | 269 | 1706 | 83 |
| | | 1 [Homo sapiens] | | | | |
| 612 | 10835109 | myotubularin-related protein 3 isoform c | 743 | 200 | 1716 | 83 |
| | | [Homo sapiens] | | | | |
| 613 | 145701025 | multiple epidermal growth factor-like | 432 | 251 | 1735 | 82 |
| | | domains protein 8 precursor [Homo sapiens] | | | | |
| 614 | 160420295 | centrosomal protein KIAA1731 [Homo sapiens] | 499 | 239 | 1798 | 80 |
| 615 | 28626521 | NFX1-type zinc finger-containing | 517 | 235 | 1816 | 80 |
| | | protein 1 [Homo sapiens] | | | | |
| 616 | 31742492 | NEDD4-binding protein 2 [Homo sapiens] | 713 | 205 | 1882 | 78 |
| 617 | 4758190 | dipeptidase 1 precursor [Homo sapiens] | 162 | 395 | 1883 | 78 |
| 618 | 24308089 | chromodomain-helicase-DNA-binding | 564 | 228 | 1888 | 77 |
| | | protein 5 [Homo sapiens] | | | | |
| 619 | 222136585 | protein timeless homolog [Homo sapiens] | 572 | 227 | 1980 | 75 |
| 620 | 171846278 | leucine-rich repeat serine/threonine- | 514 | 235 | 1983 | 75 |
| | | protein kinase 2 [Homo sapiens] | | | | |
| 621 | 21361241 | ephrin type-A receptor 3 isoform a | 528 | 234 | 2031 | 74 |
| | | precursor [Homo sapiens] | | | | |
| 622 | 164565408 | pleckstrin homology domain-containing | 748 | 200 | 2072 | 73 |
| | | family G member 2 [Homo sapiens] | | | | |
| 623 | 4505621 | phosphatidylethanolamine-binding | 233 | 332 | 2086 | 73 |
| | | protein 1 preproprotein [Homo sapiens] | | | | |
| 624 | 46049114 | kinesin-like protein KIF20B [Homo sapiens] | 270 | 312 | 2117 | 72 |
| 625 | 67782362 | ATP-dependent RNA helicase DHX29 | 510 | 237 | 2156 | 71 |
| | | [Homo sapiens] | | | | |
| 626 | 31657140 | insulin receptor-related protein | 656 | 213 | 2247 | 69 |
| | | precursor [Homo sapiens] | | | | |
| 62 | 93141226 | xaa-Pro aminopeptidase 2 precursor | 147 | 426 | 2293 | 68 |
| | | [Homo sapiens] | | | | |
| 627 | 350529351 | protein NLRC5 [Homo sapiens] | 477 | 243 | 2421 | 65 |
| 628 | 163792198 | latrophilin-3 precursor [Homo sapiens] | 729 | 202 | 2431 | 65 |
| 629 | 145046269 | rotatin [Homo sapiens] | 445 | 249 | 2476 | 64 |
| 630 | 32189398 | gastric intrinsic factor precursor | 297 | 301 | 2510 | 64 |
| | | [Homo sapiens] | | | | |
| 631 | 134133226 | POTE ankyrin domain family member E | 611 | 221 | 2624 | 61 |
| | | [Homo sapiens] | | | | |
| 632 | 115527082 | myosin-1 [Homo sapiens] | 19 | 1529 | 0 | 0 |
| 633 | 153791586 | myosin-2 [Homo sapiens] | 24 | 1461 | 0 | 0 |
| 15 | 118498350 | chymotrypsinogen B2 precursor | 25 | 1445 | 0 | 0 |
| | | [Homo sapiens] | | | | |
| 22 | 115298678 | complement C3 precursor [Homo sapiens] | 27 | 1360 | 0 | 0 |
| 634 | 125819 | RecName: Full = Ig kappa chain V-III | 45 | 898 | 0 | 0 |
| | | region HIC; Flags: Precursor | | | | |
| 635 | 4557894 | lysozyme C precursor [Homo sapiens] | 46 | 884 | 0 | 0 |
| 636 | 125801 | RecName: Full = Ig kappa chain V-III | 53 | 812 | 0 | 0 |
| | | region Ti | | | | |
| 637 | 125797 | RecName: Full = Ig kappa chain V-III | 54 | 789 | 0 | 0 |
| | | region SIE | | | | |
| 638 | 294956573 | RecName: Full = Ig lambda-6 chain C | 57 | 763 | 0 | 0 |
| | | region | | | | |
| 54 | 341913702 | PREDICTED: deleted in malignant brain tumors | 61 | 744 | 0 | 0 |
| | | 1 protein isoform 2 [Homo sapiens] | | | | |
| 639 | 4505605 | regenerating islet-derived protein | 62 | 743 | 0 | 0 |
| | | 3-alpha precursor [Homo sapiens] | | | | |
| 640 | 294956599 | RecName: Full = Ig lambda-7 chain C | 63 | 735 | 0 | 0 |
| | | region | | | | |
| 641 | 148539842 | deleted in malignant brain tumors 1 | 66 | 717 | 0 | 0 |
| | | protein isoform b precursor [Homo sapiens] | | | | |
| 28 | 4826762 | haptoglobin isoform 1 preproprotein | 68 | 692 | 0 | 0 |
| | | [Homo sapiens] | | | | |
| 642 | 123843 | RecName: Full = Ig heavy chain V-III | 76 | 591 | 0 | 0 |
| | | region VH26; Flags: Precursor | | | | |
| 643 | 4503571 | alpha-enolase isoform 1 [Homo sapiens] | 78 | 582 | 0 | 0 |
| 644 | 40354205 | fructose-bisphosphate aldolase B | 79 | 580 | 0 | 0 |
| | | [Homo sapiens] | | | | |
| 645 | 118918403 | nesprin-2 isoform 1 [Homo sapiens] | 81 | 567 | 0 | 0 |
| 646 | 113204615 | ryanodine receptor 1 isoform 1 | 84 | 564 | 0 | 0 |
| | | [Homo sapiens] | | | | |
| 647 | 125770 | RecName: Full = Ig kappa chain V-I region OU | 85 | 562 | 0 | 0 |
| 648 | 4557577 | fatty acid-binding protein liver | 88 | 560 | 0 | 0 |
| | | [Homo sapiens] | | | | |
| 649 | 125761 | RecName: Full = Ig kappa chain V-I region DEE | 92 | 549 | 0 | 0 |
| 650 | 23097308 | nesprin-1 isoform 2 [Homo sapiens] | 95 | 543 | 0 | 0 |
| 651 | 4504963 | lipocalin-1 isoform 1 precursor [Homo sapiens] | 96 | 543 | 0 | 0 |
| 652 | 58331253 | obscurin isoform a [Homo sapiens] | 97 | 542 | 0 | 0 |
| 653 | 126032350 | DNA-dependent protein kinase catalytic | 99 | 537 | 0 | 0 |
| | | subunit isoform 2 [Homo sapiens] | | | | |
| 654 | 125756 | RecName: Full = Ig kappa chain V-I region AG | 101 | 528 | 0 | 0 |
| 655 | 125774 | RecName: Full = Ig kappa chain V-I region WEA | 105 | 520 | 0 | 0 |
| 656 | 125758 | RecName: Full = Ig kappa chain V-I region AU | 106 | 517 | 0 | 0 |
| 657 | 125790 | RecName: Full = Ig kappa chain V-II | 108 | 511 | 0 | 0 |
| | | region GM607; Flags: Precursor | | | | |
| 658 | 125799 | RecName: Full = Ig kappa chain V-III | 110 | 504 | 0 | 0 |
| | | region NG9; Flags: Precursor | | | | |
| 659 | 1170720 | RecName: Full = Ig kappa chain V-I region WAT | 117 | 484 | 0 | 0 |
| 660 | 123845 | RecName: Full = Ig heavy chain V-III | 124 | 474 | 0 | 0 |
| | | region BRO | | | | |
| 661 | 125833 | RecName: Full = Ig kappa chain V-IV | 130 | 457 | 0 | 0 |
| | | region JI; Flags: Precursor | | | | |
| 662 | 257743025 | nebulin isoform 2 [Homo sapiens] | 132 | 454 | 0 | 0 |
| 663 | 123844 | RecName: Full = Ig heavy chain V-III | 134 | 453 | 0 | 0 |
| | | region TIL | | | | |
| 664 | 193806361 | RecName: Full = Ig gamma-3 chain C | 144 | 433 | 0 | 0 |
| | | region; AltName: Full = HDC; AltName: | | | | |
| | | Full = Heavy chain disease protein | | | | |
| 665 | 54607141 | vacuolar protein sorting-associated | 146 | 426 | 0 | 0 |
| | | protein 13D isoform 2 [Homo sapiens] | | | | |
| 666 | 223633991 | pantetheinase precursor [Homo sapiens] | 150 | 419 | 0 | 0 |
| 667 | 341914926 | PREDICTED: ig heavy chain V-III | 154 | 413 | 0 | 0 |
| | | region VH26-like [Homo sapiens] | | | | |
| 668 | 218512079 | RecName: Full = Ig gamma-2 chain C region | 158 | 402 | 0 | 0 |
| 669 | 125779 | RecName: Full = Ig kappa chain V-I | 161 | 397 | 0 | 0 |
| | | region Walker; Flags: Precursor | | | | |
| 670 | 125766 | RecName: Full = Ig kappa chain V-I | 167 | 391 | 0 | 0 |
| | | region HK102; Flags: Precursor | | | | |
| 671 | 125763 | RecName: Full = Ig kappa chain V-I region Gal | 171 | 388 | 0 | 0 |
| 672 | 341914862 | PREDICTED: hypothetical protein | 173 | 385 | 0 | 0 |
| | | LOC100291917 [Homo sapiens] | | | | |
| 673 | 125795 | RecName: Full = Ig kappa chain V-III | 175 | 381 | 0 | 0 |
| | | region B6 | | | | |
| 674 | 73858568 | plasma protease C1 inhibitor precursor | 178 | 377 | 0 | 0 |
| | | [Homo sapiens] | | | | |
| 675 | 341913664 | PREDICTED: hypothetical protein | 179 | 377 | 0 | 0 |
| | | LOC642424 [Homo sapiens] | | | | |
| 676 | 188595687 | filamin-C isoform b [Homo sapiens] | 180 | 376 | 0 | 0 |
| 677 | 341915168 | PREDICTED: ig kappa chain V-I region | 182 | 373 | 0 | 0 |
| | | Walker-like [Homo sapiens] | | | | |
| 678 | 256017159 | MAX gene-associated protein isoform 2 | 190 | 368 | 0 | 0 |
| | | [Homo sapiens] | | | | |
| 679 | 53759122 | adenomatous polyposis coli protein | 195 | 366 | 0 | 0 |
| | | isoform b [Homo sapiens] | | | | |
| 680 | 4502067 | protein AMBP preproprotein [Homo sapiens] | 202 | 361 | 0 | 0 |
| 681 | 47607492 | plectin isoform 1c [Homo sapiens] | 207 | 354 | 0 | 0 |
| 682 | 310114449 | PREDICTED: hypothetical protein | 210 | 351 | 0 | 0 |
| | | LOC131544 [Homo sapiens] | | | | |
| 683 | 341915514 | PREDICTED: ig kappa chain V-I region | 211 | 351 | 0 | 0 |
| | | Walker-like [Homo sapiens] | | | | |
| 684 | 125762 | RecName: Full = Ig kappa chain V-I region EU | 220 | 346 | 0 | 0 |
| 685 | 125811 | RecName: Full = Ig kappa chain V-III | 221 | 343 | 0 | 0 |
| | | region VG; Flags: Precursor | | | | |
| 686 | 312032409 | aminoacylase-1 isoform d [Homo sapiens] | 225 | 339 | 0 | 0 |
| 687 | 283806679 | cytoplasmic dynein 2 heavy chain 1 | 231 | 333 | 0 | 0 |
| | | isoform 1 [Homo sapiens] | | | | |
| 688 | 21735621 | malate dehydrogenase mitochondrial | 234 | 332 | 0 | 0 |
| | | precursor [Homo sapiens] | | | | |
| 689 | 115298657 | protein S100-A7 [Homo sapiens] | 245 | 323 | 0 | 0 |
| 690 | 27436940 | reelin isoform b precursor [Homo sapiens] | 251 | 321 | 0 | 0 |
| 691 | 51230412 | ral GTPase-activating protein subunit | 257 | 317 | 0 | 0 |
| | | alpha-1 isoform 2 [Homo sapiens] | | | | |
| 692 | 374532817 | Golgin subfamily B member 1 isoform 1 | 260 | 316 | 0 | 0 |
| | | [Homo sapiens] | | | | |
| 693 | 109826564 | neurofibromin isoform 1 [Homo sapiens] | 261 | 316 | 0 | 0 |
| 694 | 301897469 | beta-enolase isoform 1 [Homo sapiens] | 264 | 315 | 0 | 0 |
| 695 | 38683816 | ankyrin repeat domain-containing | 276 | 308 | 0 | 0 |
| | | protein 17 isoform b [Homo sapiens] | | | | |
| 696 | 16579888 | fructose-16-bisphosphatase 1 [Homo sapiens] | 284 | 306 | 0 | 0 |
| 697 | 126362967 | nck-associated protein 5 isoform 1 | 287 | 305 | 0 | 0 |
| | | [Homo sapiens] | | | | |
| 698 | 38045890 | CUB and sushi domain-containing | 301 | 299 | 0 | 0 |
| | | protein 3 isoform 2 [Homo sapiens] | | | | |
| 699 | 331284178 | nuclear receptor corepressor 2 isoform 1 | 309 | 292 | 0 | 0 |
| | | [Homo sapiens] | | | | |
| 700 | 151301154 | mucin-6 precursor [Homo sapiens] | 311 | 291 | 0 | 0 |
| 701 | 298919181 | nuclear receptor corepressor 1 isoform 3 | 321 | 285 | 0 | 0 |
| | | [Homo sapiens] | | | | |
| 702 | 27477095 | histone-lysine N-methyltransferase H3 | 331 | 281 | 0 | 0 |
| | | lysine-36 and H4 lysine-20 specific | | | | |
| | | isoform a [Homo sapiens] | | | | |
| 703 | 98985810 | collagen alpha-1(XI) chain isoform B | 334 | 280 | 0 | 0 |
| | | preproprotein [Homo sapiens] | | | | |
| 704 | 269954694 | inositol 1 4 5-trisphosphate receptor | 339 | 279 | 0 | 0 |
| | | type 1 isoform 3 [Homo sapiens] | | | | |
| 705 | 66346674 | vacuolar protein sorting-associated | 343 | 278 | 0 | 0 |
| | | protein 13A isoform A [Homo sapiens] | | | | |
| 706 | 125786 | RecName: Full = Ig kappa chain V-II region MIL | 349 | 274 | 0 | 0 |
| 707 | 38505274 | voltage-dependent T-type calcium channel | 355 | 272 | 0 | 0 |
| | | subunit alpha-1G isoform 7 [Homo sapiens] | | | | |
| 708 | 148536846 | voltage-dependent P/Q-type calcium channel | 358 | 271 | 0 | 0 |
| | | subunit alpha-1A isoform 2 [Homo sapiens] | | | | |
| 709 | 5032281 | dystrophin Dp427c isoform [Homo sapiens] | 379 | 266 | 0 | 0 |
| 710 | 122937195 | perilipin-4 [Homo sapiens] | 381 | 265 | 0 | 0 |
| 711 | 209969819 | protein PRR14L [Homo sapiens] | 383 | 264 | 0 | 0 |
| 712 | 341915607 | PREDICTED: hypothetical protein | 389 | 263 | 0 | 0 |
| | | LOC144535 [Homo sapiens] | | | | |
| 713 | 33356170 | myosin-IXb isoform 1 [Homo sapiens] | 390 | 263 | 0 | 0 |
| 714 | 59710093 | probable G-protein coupled receptor 112 | 392 | 263 | 0 | 0 |
| | | [Homo sapiens] | | | | |
| 715 | 341913933 | PREDICTED: hypothetical protein | 400 | 262 | 0 | 0 |
| | | LOC100653084 [Homo sapiens] | | | | |
| 716 | 341914924 | PREDICTED: ig heavy chain V-III | 405 | 260 | 0 | 0 |
| | | region VH26-like [Homo sapiens] | | | | |
| 717 | 116089331 | transcription factor HIVEP3 isoform a | 406 | 260 | 0 | 0 |
| | | [Homo sapiens] | | | | |
| 718 | 224586880 | ras-specific guanine nucleotide-releasing | 415 | 257 | 0 | 0 |
| | | factor 1 isoform 3 [Homo sapiens] | | | | |
| 719 | 113415686 | PREDICTED: hypothetical protein | 420 | 256 | 0 | 0 |
| | | LOC285556 [Homo sapiens] | | | | |
| 720 | 311771583 | LY75-CD302 fusion protein isoform 1 | 437 | 250 | 0 | 0 |
| | | precursor [Homo sapiens] | | | | |
| 721 | 214010175 | CLIP-associating protein 1 isoform 3 | 443 | 249 | 0 | 0 |
| | | [Homo sapiens] | | | | |
| 722 | 16933542 | fibronectin isoform 3 preproprotein | 446 | 249 | 0 | 0 |
| | | [Homo sapiens] | | | | |
| 723 | 31563537 | CLIP-associating protein 1 isoform 1 | 447 | 249 | 0 | 0 |
| | | [Homo sapiens] | | | | |
| 724 | 73695475 | HEAT repeat-containing protein 1 | 449 | 248 | 0 | 0 |
| | | [Homo sapiens] | | | | |
| 725 | 5803011 | gamma-enolase [Homo sapiens] | 451 | 248 | 0 | 0 |
| 726 | 296317312 | carcinoembryonic antigen-related cell | 460 | 247 | 0 | 0 |
| | | adhesion molecule 1 isoform 3 precursor | | | | |
| | | [Homo sapiens] | | | | |
| 727 | 117168250 | 1-phosphatidylinositol-4 5-bisphosphate | 463 | 246 | 0 | 0 |
| | | phosphodiesterase epsilon-1 isoform 1 | | | | |
| | | [Homo sapiens] | | | | |
| 728 | 363807222 | girdin isoform 3 [Homo sapiens] | 464 | 246 | 0 | 0 |
| 729 | 121114300 | carcinoembryonic antigen-related cell | 466 | 245 | 0 | 0 |
| | | adhesion molecule 3 precursor | | | | |
| | | [Homo sapiens] | | | | |
| 730 | 125809 | RecName: Full = Ig kappa chain V-III | 469 | 245 | 0 | 0 |
| | | region CLL; AltName: Full = Rheumatoid | | | | |
| | | factor; Flags: Precursor | | | | |
| 731 | 170650694 | arf-GAP with GTPase ANK repeat and | 474 | 243 | 0 | 0 |
| | | PH domain-containing protein 2 isoform | | | | |
| | | PIKE-L [Homo sapiens] | | | | |
| 732 | 116008192 | myosin light chain kinase smooth | 478 | 243 | 0 | 0 |
| | | muscle isoform 1 [Homo sapiens] | | | | |
| 733 | 33620769 | E3 ubiquitin-protein ligase RBBP6 | 481 | 243 | 0 | 0 |
| | | isoform 1 [Homo sapiens] | | | | |
| 734 | 4502895 | colipase isoform 1 preproprotein | 482 | 243 | 0 | 0 |
| | | [Homo sapiens] | | | | |
| 735 | 4505941 | DNA-directed RNA polymerase II | 484 | 242 | 0 | 0 |
| | | subunit RPB2 [Homo sapiens] | | | | |
| 736 | 154759259 | spectrin alpha chain brain isoform 2 | 488 | 242 | 0 | 0 |
| | | [Homo sapiens] | | | | |
| 737 | 208022632 | girdin isoform 1 [Homo sapiens] | 489 | 241 | 0 | 0 |
| 738 | 350276222 | neuron navigator 2 isoform 5 [Homo sapiens] | 490 | 241 | 0 | 0 |
| 739 | 15890086 | collagen alpha-5(IV) chain isoform 2 | 523 | 234 | 0 | 0 |
| | | precursor [Homo sapiens] | | | | |
| 740 | 333440451 | CLIP-associating protein 2 isoform 2 | 524 | 234 | 0 | 0 |
| | | [Homo sapiens] | | | | |
| 741 | 327365361 | HEAT repeat-containing protein 5A | 527 | 234 | 0 | 0 |
| | | [Homo sapiens] | | | | |
| 742 | 92091583 | myosin-11 isoform SM2B [Homo sapiens] | 529 | 234 | 0 | 0 |
| 743 | 215598574 | ankyrin-1 isoform 9 [Homo sapiens] | 534 | 233 | 0 | 0 |
| 744 | 20336209 | transcriptional regulator ATRX isoform | 539 | 232 | 0 | 0 |
| | | 1 [Homo sapiens] | | | | |
| 745 | 24308029 | dedicator of cytokinesis protein 9 | 541 | 232 | 0 | 0 |
| | | isoform a [Homo sapiens] | | | | |
| 746 | 123847 | RecName: Full = Ig heavy chain V-III | 545 | 232 | 0 | 0 |
| | | region CAM | | | | |
| 747 | 161169013 | neuron navigator 2 isoform 1 [Homo sapiens] | 552 | 231 | 0 | 0 |
| 748 | 4557365 | Bloom syndrome protein [Homo sapiens] | 553 | 231 | 0 | 0 |
| 749 | 46049105 | nebulin-related-anchoring protein | 561 | 228 | 0 | 0 |
| | | isoform S [Homo sapiens] | | | | |
| 750 | 4504349 | hemoglobin subunit beta [Homo sapiens] | 570 | 228 | 0 | 0 |
| 751 | 93204888 | spatacsin isoform 1 [Homo sapiens] | 573 | 227 | 0 | 0 |
| 752 | 123846 | RecName: Full = Ig heavy chain V-III | 575 | 227 | 0 | 0 |
| | | region BUT | | | | |
| 753 | 120953300 | leucine-rich repeat and IQ domain- | 577 | 227 | 0 | 0 |
| | | containing protein 1 [Homo sapiens] | | | | |
| 754 | 311771516 | myomegalin isoform 8 [Homo sapiens] | 579 | 226 | 0 | 0 |
| 755 | 114155142 | nucleoprotein TPR [Homo sapiens] | 582 | 226 | 0 | 0 |
| 756 | 71274186 | uncharacterized protein KIAA1755 | 583 | 225 | 0 | 0 |
| | | [Homo sapiens] | | | | |
| 757 | 70780355 | ankyrin-1 isoform 2 [Homo sapiens] | 591 | 225 | 0 | 0 |
| 758 | 4503689 | fibrinogen alpha chain isoform alpha-E | 593 | 224 | 0 | 0 |
| | | preproprotein [Homo sapiens] | | | | |
| 759 | 4506773 | protein S100-A9 [Homo sapiens] | 595 | 224 | 0 | 0 |
| 760 | 355390328 | kinesin-like protein KIF21B isoform 3 | 609 | 221 | 0 | 0 |
| | | [Homo sapiens] | | | | |
| 761 | 315709510 | protein dopey-1 isoform b [Homo sapiens] | 612 | 221 | 0 | 0 |
| 762 | 224831241 | myosin-14 isoform 3 [Homo sapiens] | 615 | 220 | 0 | 0 |
| 763 | 22094135 | histone-lysine N-methyltransferase H3 | 624 | 219 | 0 | 0 |
| | | lysine-79 specific [Homo sapiens] | | | | |
| 764 | 300797780 | serine/threonine-protein kinase WNK1 | 626 | 218 | 0 | 0 |
| | | isoform 3 [Homo sapiens] | | | | |
| 765 | 256818778 | protein unc-80 homolog isoform 2 | 633 | 217 | 0 | 0 |
| | | [Homo sapiens] | | | | |
| 766 | 125777 | RecName: Full = Ig kappa chain V-I region Ni | 639 | 216 | 0 | 0 |
| 767 | 21361861 | Fanconi anemia group D2 protein | 641 | 215 | 0 | 0 |
| | | isoform a [Homo sapiens] | | | | |
| 768 | 293597572 | transient receptor potential cation | 647 | 214 | 0 | 0 |
| | | channel subfamily M member 6 isoform | | | | |
| | | b [Homo sapiens] | | | | |
| 769 | 41393563 | kinesin-like protein KIF1B isoform b | 648 | 214 | 0 | 0 |
| | | [Homo sapiens] | | | | |
| 770 | 267844813 | neuron navigator 1 isoform 2 [Homo sapiens] | 649 | 213 | 0 | 0 |
| 771 | 269847874 | probable ATP-dependent RNA helicase | 662 | 212 | 0 | 0 |
| | | YTHDC2 [Homo sapiens] | | | | |
| 772 | 222352161 | probable phospholipid-transporting | 666 | 211 | 0 | 0 |
| | | ATPase VD [Homo sapiens] | | | | |
| 773 | 65287717 | eukaryotic translation initiation factor 2- | 667 | 211 | 0 | 0 |
| | | alpha kinase 4 [Homo sapiens] | | | | |
| 774 | 224586815 | Golgi apparatus protein 1 isoform 2 | 673 | 210 | 0 | 0 |
| | | precursor [Homo sapiens] | | | | |
| 775 | 94966754 | elongation factor Tu GTP-binding | 677 | 209 | 0 | 0 |
| | | domain-containing protein 1 isoform 1 | | | | |
| | | [Homo sapiens] | | | | |
| 776 | 115648142 | centrosomal protein of 164 kDa | 680 | 209 | 0 | 0 |
| | | [Homo sapiens] | | | | |
| 777 | 46852172 | kinesin-like protein KIF13B [Homo sapiens] | 681 | 209 | 0 | 0 |
| 778 | 19913410 | major vault protein [Homo sapiens] | 683 | 209 | 0 | 0 |
| 58 | 58331211 | chymotrypsin-like elastase family | 687 | 208 | 0 | 0 |
| | | member 2B preproprotein [Homo sapiens] | | | | |
| 779 | 38044112 | CAP-Gly domain-containing linker | 688 | 208 | 0 | 0 |
| | | protein 1 isoform b [Homo sapiens] | | | | |
| 780 | 40255013 | carcinoembryonic antigen-related cell | 689 | 208 | 0 | 0 |
| | | adhesion molecule 6 precursor | | | | |
| | | [Homo sapiens] | | | | |
| 781 | 4504919 | keratin type II cytoskeletal 8 isoform 2 | 691 | 208 | 0 | 0 |
| | | [Homo sapiens] | | | | |
| 782 | 194097325 | fatty acid-binding protein intestinal | 701 | 206 | 0 | 0 |
| | | [Homo sapiens] | | | | |
| 783 | 319803120 | testis-expressed protein 14 isoform c | 702 | 206 | 0 | 0 |
| | | [Homo sapiens] | | | | |
| 784 | 354721145 | transient receptor potential cation | 707 | 206 | 0 | 0 |
| | | channel subfamily M member 1 isoform | | | | |
| | | 1 [Homo sapiens] | | | | |
| 785 | 151301137 | AT-hook-containing transcription factor | 711 | 205 | 0 | 0 |
| | | [Homo sapiens] | | | | |
| 786 | 45827771 | enhancer of mRNA-decapping protein 4 | 712 | 205 | 0 | 0 |
| | | [Homo sapiens] | | | | |
| 787 | 208609951 | neurexin-1-beta isoform alpha2 | 716 | 204 | 0 | 0 |
| | | precursor [Homo sapiens] | | | | |
| 788 | 217416354 | A-kinase anchor protein SPHKAP | 719 | 203 | 0 | 0 |
| | | isoform 1 [Homo sapiens] | | | | |
| 789 | 30794372 | protein polybromo-1 isoform 1 [Homo sapiens] | 725 | 202 | 0 | 0 |
| 790 | 24307991 | cullin-9 [Homo sapiens] | 726 | 202 | 0 | 0 |
| 791 | 50345997 | histone acetyltransferase p300 [Homo sapiens] | 735 | 201 | 0 | 0 |
| 792 | 149773449 | zinc finger protein 862 [Homo sapiens] | 736 | 201 | 0 | 0 |
| 479 | 38327601 | regulator of G-protein signaling 12 | 737 | 201 | 0 | 0 |
| | | isoform 1 [Homo sapiens] | | | | |
| 43 | 42794779 | myosin-XVIIIa isoform b [Homo sapiens] | 745 | 200 | 0 | 0 |
| 67 | 157419122 | laminin subunit alpha-4 isoform 2 | 747 | 200 | 0 | 0 |
| | | precursor [Homo sapiens] | | | | |

The NCBI Accession Numbers for proteins defined by the NCBI protein database has been provided. The sequences of the proteins as reflected by the NCBI Accession Numbers listed throughout the present application are incorporated herein by reference. Where a protein is named in its preprotein or other non-mature form, the mature form of the protein is equally implied including such changes as removal of signal sequences and the addition of post-translational modifications. In all cases, the protein has been named by its gene derived sequence to provide consistency. In addition, isoforms of each of the proteins identified herein are similarly envisioned.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

1. A method of assessing whether a subject is afflicted with pancreatic cancer, the method comprising
determining the level of at least one pancreatic cancer biomarker in a sample derived from said subject; and
comparing the level of the pancreatic cancer biomarker with the level of the pancreatic cancer biomarker in a control sample, wherein a difference between the level of the pancreatic cancer biomarker derived from said subject and the pancreatic cancer biomarker in the control sample is an indication that the subject is afflicted with pancreatic cancer.

2. The method of claim 1, wherein the pancreatic cancer biomarker is
(a) CA 19-9,
(b) a protein comprising an amino acid sequence selected from the group consisting of SEQ ID NOs: 1-31 or 39-793, or a fragment thereof,
(c) a protein comprising an amino acid sequence selected from the group consisting of SEQ ID NOs: 1-19, 47, 49, 55-58, 206, 726, 729, 780 or 793, or a fragment thereof, or
(d) a nucleotide sequence encoding the protein.

3-4. (canceled)

5. The method of claim 1, wherein the sample is selected from the group consisting of a fecal sample, a gastrointestinal lavage fluid, and a combination thereof.

6. (canceled)

7. The method of claim 1, comprising determining the level of at least 2, 3, 4, 6, 7, 8, 9 or 10 pancreatic cancer biomarkers and comparing the level of each of the pancreatic cancer biomarkers to the respective level of the pancreatic cancer biomarkers in the control sample.

8. (canceled)

9. The method of claim 1, wherein the subject is a human.

10. The method of claim 1, further comprising administering a lavage fluid and collecting the sample, optionally
(a) wherein the sample is a gastrointestinal lavage fluid,
(b) wherein the lavage fluid is administered orally,
(c) wherein the lavage fluid comprises an ingredient selected from the group consisting of polyethylene glycol, magnesium sulfate, sodium sulfate, potassium sulfate, magnesium citrate, ascorbic acid, sodium picosulfate, and bisacodyl, and/or
(d) wherein the lavage fluid is selected from the group consisting of GOLYTELY, HALFLYTELY, NULYTELY, SUPREP, FLEET'S PHOSPHO-SODA, magnesium citrate, and their generic equivalents.

11-14. (canceled)

15. The method of claim 1, further comprising partially purging the subject's gastrointestinal system and collecting gastrointestinal lavage fluid.

16. The method of claim 1, wherein the difference is a decrease in the level of the pancreatic cancer biomarker derived from said subject and the pancreatic cancer biomarker in the control sample, and wherein said decrease is an indication that the subject is afflicted with pancreatic cancer, optionally, wherein the level of the pancreatic cancer biomarker derived from said subject is at least 3, 5, 10 or 100 times less than the level of the pancreatic cancer biomarker in the control sample.

17-18. (canceled)

19. The method of claim 16, wherein the pancreatic cancer biomarker is a protein encoded by an amino acid sequence selected from the group consisting of SEQ ID NOs:1-16, 49, 55-58, 206 and 793, or a fragment thereof.

20. The method of claim 1, wherein the difference is an increase in the level of the pancreatic cancer biomarker derived from said subject and the pancreatic cancer biomarker in the control sample, and wherein said increase is an indication that the subject is afflicted with pancreatic cancer, optionally, wherein the level of the pancreatic cancer biomarker derived from said subject is at least 3, 5, 10 or 100 times more than the level of the pancreatic cancer biomarker in the control sample.

21-22. (canceled)

23. The method of claim 20, wherein the pancreatic cancer biomarker is CA19-9 or is a protein encoded by an amino acid sequence selected from the group consisting of SEQ ID NOs:17-19, 47, 726, 729 or 780, or a fragment thereof.

24. The method of claim 1, wherein the pancreatic cancer biomarker is derived from the pancreas.

25. The method of claim 1, wherein the pancreatic cancer is selected from the group consisting of an exocrine pancreatic cancer, a pancreatic cystic neoplasm and a pancreatic endocrine cancer, optionally, wherein
(a) the exocrine pancreatic cancer is selected from the group consisting of pancreatic ductal adenocarcinoma (PDAC), adenosquamous carcinoma, squamous cell carcinoma, giant cell carcinoma, acinar cell carcinoma and small cell carcinoma, or
(b) the pancreatic endocrine cancer is selected from the group consisting of insulinomas, glucagonomas, somatostatinomas, gastrinomas, VlPomas and non-secreting islet tumors of the pancreas.

26-28. (canceled)

29. The method of claim 1, wherein determining the level of said at least pancreatic cancer biomarker comprises
(a) performing an immunoassay or a colorimetric assay, optionally, wherein the immunoassay m the group consisting of a Western blot, an enzyme linked immunoabsorbent assay (ELISA), and a radioimmunoassay,
(b) performing mass spectrometry, or
(c) applying said sample to a solid phase test strip or a flow-through strip comprising an agent which selectively binds to said pancreatic cancer biomarker; and
detecting said pancreatic cancer biomarker bound to said agent on said solid phase test strip or said flow-through strip.

30-33. (canceled)

34. The method of claim 1, further comprising comparing the level of the pancreatic cancer biomarker from the subject with the level of at least one control polypeptide, or fragment thereof, or a nucleic acid encoding said at least one control polypeptide, derived from the sample, optionally wherein the control polypeptide
(a) is a non-pancreatic polypeptide that originates in the gastrointestinal tract, or
(b) is CA19-19 or comprises an amino acid sequence selected from the group consisting of SEQ ID NOs: 27, 32-40, 45, 54, and 59, or a fragment thereof.

35-36. (canceled)

37. A method of assessing the progression of pancreatic cancer in a subject afflicted with pancreatic cancer, the method comprising
determining the level of at least one pancreatic cancer biomarker in a sample derived from said subject; and
comparing the level of the pancreatic cancer biomarker with the level of the pancreatic cancer biomarker in a control sample, wherein a decrease in the level of the pancreatic cancer biomarker derived from said subject and the pancreatic cancer biomarker in the control sample is an indication that the pancreatic cancer will progress rapidly; and wherein an increase in the level of the pancreatic cancer biomarker derived from said subject and the pancreatic cancer biomarker in the control sample is an indication that the pancreatic cancer will progress slowly or will regress;
optionally, wherein the pancreatic cancer biomarker is CA19-9, a protein encoded by an amino acid sequence selected from the group consisting of SEQ ID NOs:1-31 or 39-793, a fragment thereof, or a nucleotide sequence encoding the protein or fragment thereof.

38. The method of claim 37, wherein the pancreatic cancer biomarker is
(a) a protein encoded by an amino acid sequence selected from the group consisting of SEQ ID NOs:1-16, 49, 55-58, 206 and 793, or a fragment thereof, or
(b) a nucleotide sequence encoding the protein or the fragment thereof.

39. (canceled)

40. A method of assessing the progression of pancreatic cancer in a subject afflicted with pancreatic cancer, the method comprising
determining the level of at least one pancreatic cancer biomarker in a sample derived from said subject; and
comparing the level of the pancreatic cancer biomarker with the level of the pancreatic cancer biomarker in a control sample, wherein an increase in the level of the pancreatic cancer biomarker derived from said subject and the pancreatic cancer biomarker in the control sample is an indication that the pancreatic cancer will progress rapidly; and wherein a decrease in the level of the pancreatic cancer biomarker derived from said subject and the pancreatic cancer biomarker in the control sample is an indication that the pancreatic cancer will progress slowly or will regress;
optionally, wherein the pancreatic cancer biomarker is CA19-9, a protein encoded by an amino acid sequence selected from the group consisting of SEQ ID NOs:1-31 or 39-793, a fragment thereof, or a nucleotide sequence encoding the protein or fragment thereof.

41. The method of claim 40, wherein the pancreatic cancer biomarker is
(a) CA19-9 or is a protein encoded by an amino acid sequence selected from the group consisting of SEQ ID NOs:17-19, 47, 726, 729 or 780, or a fragment thereof, or
(b) a nucleotide sequence encoding the protein or the fragment thereof.

42-66. (canceled)

67. A method of monitoring the efficacy of treatment of pancreatic cancer in a subject suffering from pancreatic cancer, the method comprising
determining the level of at least one pancreatic cancer biomarker in a sample derived from said subject, wherein said subject has been previously exposed to treatment for pancreatic cancer; and
comparing the level of the pancreatic cancer biomarker with the level of the pancreatic cancer biomarker in a control sample, wherein a decrease in the level of the pancreatic cancer biomarker derived from said subject and the pancreatic cancer biomarker in the control sample is an indication that the treatment is not efficacious; and wherein an increase in the level of the pancreatic cancer biomarker derived from said subject and the pancreatic cancer biomarker in the control sample is an indication that the pancreatic cancer is efficacious;
optionally, wherein the pancreatic cancer biomarker is CA19-9, a protein encoded by an amino acid sequence selected from the group consisting of SEQ ID NOs:1-31 or 39-793, a fragment thereof, or a nucleotide sequence encoding the protein or fragment thereof.

68. The method of claim 67, wherein the pancreatic cancer biomarker is
(a) a protein encoded by an amino acid sequence selected from the group consisting of SEQ ID NOs:1-16, 49, 55-58, 206 and 793, or a fragment thereof, or
(b) a nucleotide sequence encoding the protein or the fragment thereof.

69. (canceled)

70. A method of monitoring the efficacy of treatment of pancreatic cancer in a subject suffering from pancreatic cancer, the method comprising
determining the level of at least one pancreatic cancer biomarker in a sample derived from said subject, wherein said subject has been previously exposed to treatment for pancreatic cancer; and
comparing the level of the pancreatic cancer biomarker with the level of the pancreatic cancer biomarker in a control sample, wherein an increase in the level of the pancreatic cancer biomarker derived from said subject and the pancreatic cancer biomarker in the control sample is an indication that the treatment is not efficacious; and wherein a decrease in the level of the pancreatic cancer biomarker derived from said subject and the pancreatic cancer biomarker in the control sample is an indication that the pancreatic cancer is efficacious;
optionally, wherein the pancreatic cancer biomarker is CA19-9, a protein encoded by an amino acid sequence selected from the group consisting of SEQ ID NOs:1-31 or 39-793, a fragment thereof, or a nucleotide sequence encoding the protein or fragment thereof.

71. The method of claim 70, wherein the pancreatic cancer biomarker is
(a) CA19-9 or is a protein encoded by an amino acid sequence selected from the group consisting of SEQ ID NOs:17-19, 47, 726, 729 or 780, or a fragment thereof, or
(b) a nucleotide sequence encoding the protein or the fragment thereof.

72. (canceled)

73. A method of treating a subject having pancreatic cancer, the method comprising
determining the level of at least one pancreatic cancer biomarker in a sample derived from said subject;
comparing the level of the pancreatic cancer biomarker with the level of the pancreatic cancer biomarker in a control sample, wherein a difference between the level of the pancreatic cancer biomarker derived from said subject and the pancreatic cancer biomarker in the control sample is an indication that the subject is afflicted with pancreatic cancer; and
exposing said subject to therapeutically effective treatment, thereby treating the subject having pancreatic cancer.

74. A kit for determining the presence, absence or progression of pancreatic cancer in a subject comprising an agent that selectively binds to at least one pancreatic cancer biomarker.

75. The kit of claim 74, wherein the pancreatic cancer biomarker is
(a) a protein comprising an amino acid sequence selected from the group consisting of SEQ ID NOs: 1-31 or 39-793, or a fragment thereof, or
(b) a nucleotide sequence encoding the protein or the fragment thereof.

76-85. (canceled)
